(19) United States
(12) Patent Application Publication
(10) Pub. No.: US 2026/0231673 A1
(43) Pub. Date: Aug. 6, 2026

(54) MATERIALS FOR ELECTRONIC DEVICES

(71) Applicant: Merck Patent GMBH, Darmstadt (DE)

(72) Inventors: Rouven Linge, Darmstadt (DE); Elvira Montenegro, Weinheim (DE)

(21) Appl. No.: 19/133,541

(22) PCT Filed: Dec. 11, 2023
(86) PCT No.: PCT/EP2023/085018

§ 371 Date: May 28, 2025

(30) Foreign Application Priority Data
Dec. 12, 2022 (EP) 22212783.9

(51) Int. Cl. H10K 85/60; C07B 59/00; C07D 307/94; H10K 50/11; H10K 50/15

(52) U.S. Cl. CPC H10K 85/633; C07B 59/002; C07D 307/94; H10K 85/636; H10K 50/11; H10K 50/15; H10K 85/615; H10K 85/626; H10K 85/6574

(57) ABSTRACT

The present invention relates to compounds of a formula (I) or (II), processes for preparing compounds of this type, electronic devices, in particular OLEDs, containing one or more of these compounds, and the use of these compounds in electronic devices, in particular in OLEDs.

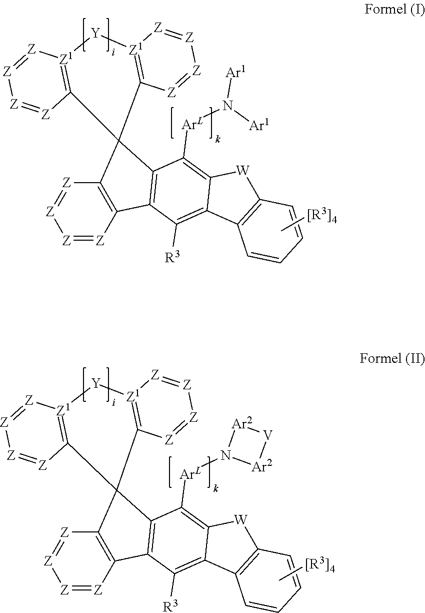

The present application relates to aromatic amines having particular aromatic or heteroaromatic ring systems on the amine nitrogen atom. The compounds are suitable for use in electronic devices.

Electronic devices in the context of this application are understood to mean what are called organic electronic devices, which comprise organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The structure and general principle of function of OLEDs are known to those skilled in the art.

In electronic devices, especially OLEDs, there is great interest in an improvement in the performance data. In these aspects, it has not yet been possible to find any entirely satisfactory solution.

A major influence on the performance data of electronic devices is possessed by emission layers and layers having a hole-transporting function. There is an ongoing search for novel compounds for use in these layers, especially hole-transporting compounds and compounds that can serve as hole-transporting matrix material, especially for phosphorescent emitters, in an emitting layer. For this purpose, there is a search in particular for compounds that have a high glass transition temperature, high stability, and high conductivity for holes. A high stability of the compound is a prerequisite for achieving a long lifetime of the electronic device. There is also a search for compounds whose use in electronic devices results in improvement of the performance data of the devices, especially in high efficiency, long lifetime and low operating voltage.

In the prior art, triarylamine compounds in particular, for example spirobifluoreneamines and fluoreneamines, are known as hole transport materials and hole-transporting matrix materials for electronic devices.

However, there remains room for improvement in respect of the abovementioned properties.

It has now been found that aromatic amines of the formulae below which are characterized in that they have particular aromatic or heteroaromatic ring systems on the amine nitrogen atom are of excellent suitability for use in electronic devices. They are especially suitable for use in OLEDs, and even more particularly therein for use as hole transport materials and for use as hole-transporting matrix materials, especially for phosphorescent emitters. The compounds lead to high lifetime, high efficiency and low operating voltage of the devices. Further preferably, the compounds found have a high glass transition temperature, high stability, low sublimation temperature, good solubility, good synthetic accessibility and high conductivity for holes.

The present application thus provides a compound of one of the following formulae:

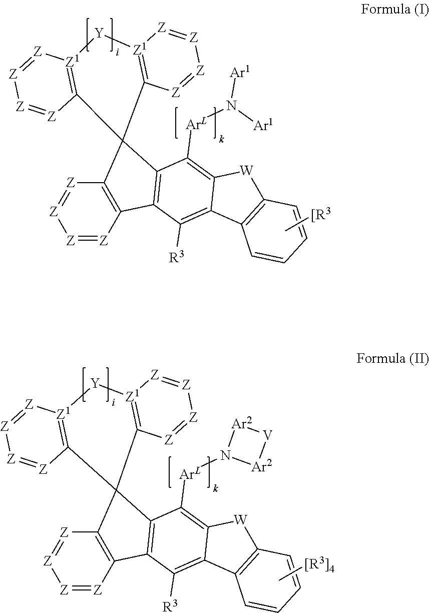

where:
W is the same or different at each instance and is selected from O and S, preferably O;
Z is the same or different at each instance and is selected from CR¹ and N, preferably CR¹;
i is 0 or 1, where the Y group is absent when i=0;
when i=0, Z¹ is the same or different at each instance and is selected from CR¹ and N;
when i=1, Z¹ is C;
Y is the same or different at each instance and is selected from a bond, C(R¹)₂, O and S;
Ar^{L} is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
k is 0, 1, 2 or 3, where, when k=0, the Ar^{L} group is absent and the two groups that bind to Ar^{L} in formula (I) and (II) are bonded directly to one another, where, when k=2, two Ar^{L} groups are bonded successively in a chain, and where, when k=3, three Ar^{L} groups are bonded successively in a chain;
Ar¹ is the same or different at each instance and is selected from aromatic ring systems which have 6 to 40 aromatic ring atoms and are substituted by R⁴ radicals, and heteroaromatic ring systems which have 5 to 40 aromatic ring atoms and are substituted by R⁴ radicals;
Ar² is the same or different at each instance and is selected from aromatic ring systems which have 6 to 40 aromatic ring atoms and are substituted by R⁵ radicals, and heteroaromatic ring systems which have 5 to 40 aromatic ring atoms and are substituted by R⁵ radicals, where the aromatic or heteroaromatic ring systems are joined in the ortho position to the V group and the nitrogen atom;
V is the same or different at each instance and is selected from a bond, O, S, Si(R⁵)₂ and C(R⁵)₂;
R¹ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R² is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R³ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁴ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁴ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, C(═O)R⁷, CN, Si(R⁷)₃, N(R⁷)₂, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁶ radicals may be joined to one another and may form a ring; where the alkyl, alkoxy, alkenyl and alkynyl groups mentioned and the aromatic ring systems and heteroaromatic ring systems mentioned are each substituted by R⁷ radicals; and where one or more CH₂ groups in the alkyl, alkoxy, alkenyl and alkynyl groups mentioned may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
R⁷ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁷ radicals may be joined to one another and may form a ring; and where the alkyl, alkoxy, alkenyl and alkynyl groups, aromatic ring systems and heteroaromatic ring systems mentioned may be substituted by one or more radicals selected from F and CN.

The representation

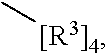

shown in a ring, is understood to mean that one R³ radical is bonded to each of the four free positions on the ring, where the R³ radicals may be the same or different at each instance.

For similar representations, for example

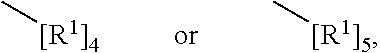

corresponding definitions are applicable, where the R¹ radical here is bonded to the ring in question and there are four or five free positions to which the radicals are bonded, which may be the same or different at each instance.

The definitions which follow are applicable to the chemical groups that are used in the present application. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms. In addition, an aryl group does not contain any heteroatom as aromatic ring atom, but only carbon atoms.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more nonaromatic rings fused to at least one aryl group. These nonaromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more nonaromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a nonaromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be substituted by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical is bonded to the position to which the hydrogen atom was bonded, forming a ring.

In a preferred embodiment, Z is CR¹. In an alternative preferred embodiment, Z is the same or different at each instance and are selected from CR¹ and N, where not more than one Z group ring is N.

The index i is 0 or 1, where the Y group is absent when i=0 and the Z¹ group is the same or different at each instance and is selected from CR¹ and N. Preferably, Z¹ when i=0 is CR¹.

If i=1, the Y group is present and is the same or different at each instance and is selected from a bond, C(R¹)₂, O or S, preferably a bond or C(R¹)₂, more preferably a bond.

In a particularly preferred configuration, i=1 and Y is a bond. In this configuration, a spirobifluorene skeleton is formed. It is also preferred over other executions in which i=1 that i=0 and Z¹ is CR¹. However, the configuration in which i=1 and Y is a bond is preferred over the execution in which i=0 and Z¹ is CR¹.

In a preferred embodiment, W at each instance is O.

Ar^{L} is the same or different at each instance and is selected from aromatic ring systems which have 6 to 25 aromatic ring atoms and are substituted by R² radicals, and heteroaromatic ring systems which have 5 to 25 aromatic ring atoms and are substituted by R² radicals; and is more preferably the same or different at each instance and is selected from phenyl, biphenyl, naphthyl and fluorenyl, each substituted by R² radicals; and is most preferably selected from phenyl substituted by R² radicals.

Preferably, Ar^{L} is the same or different at each instance and is selected from groups of the following formulae:

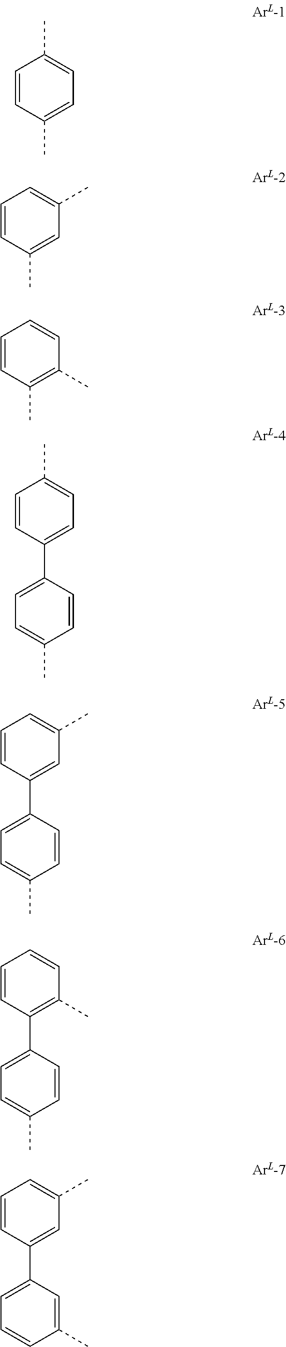

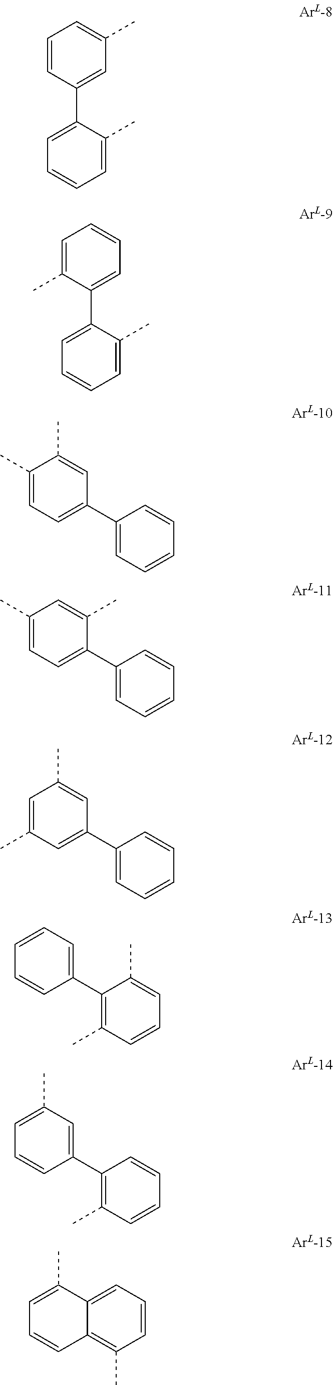

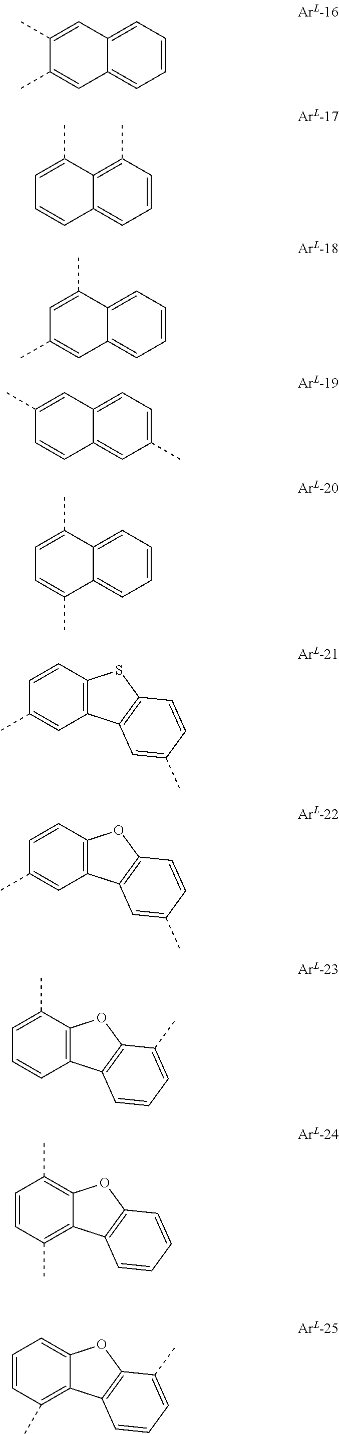

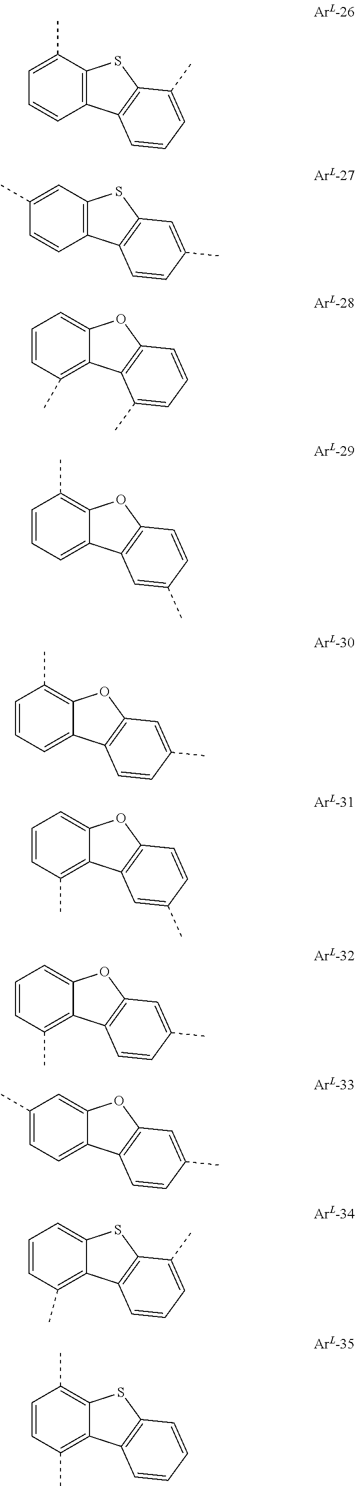

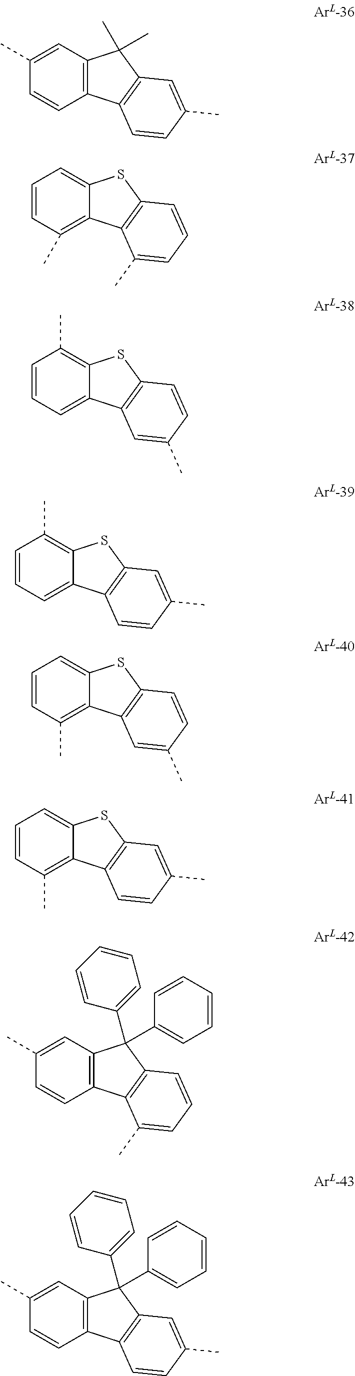

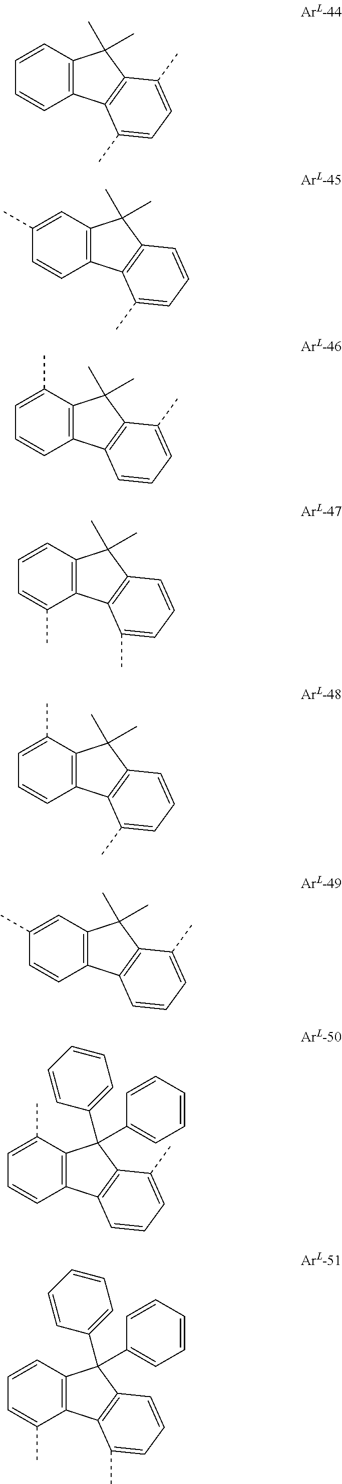

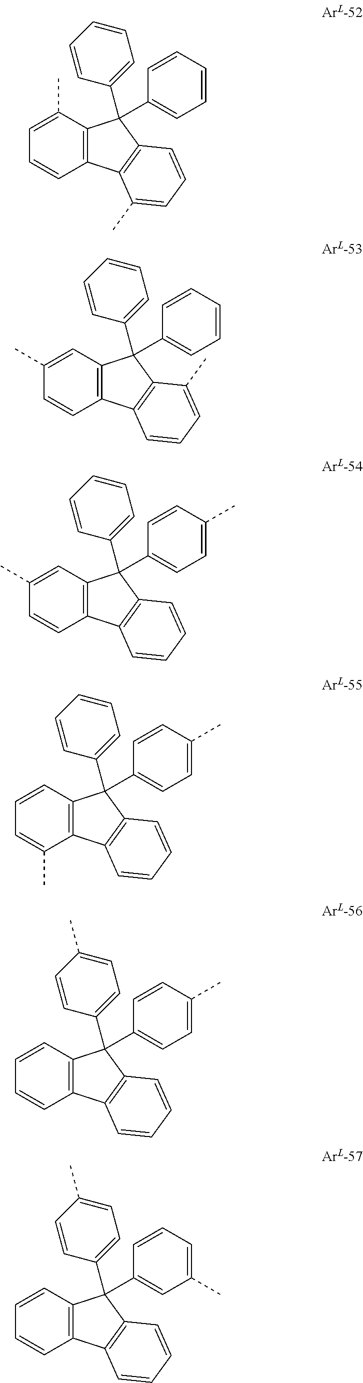

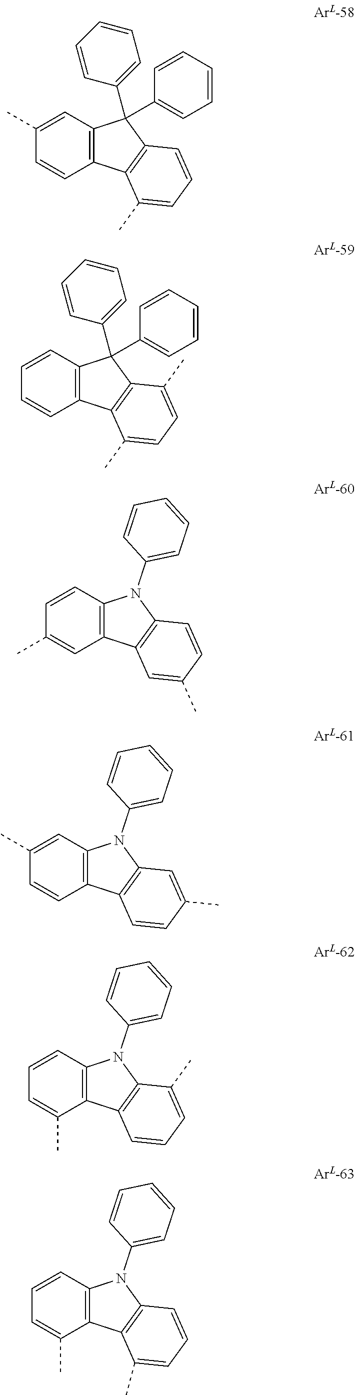

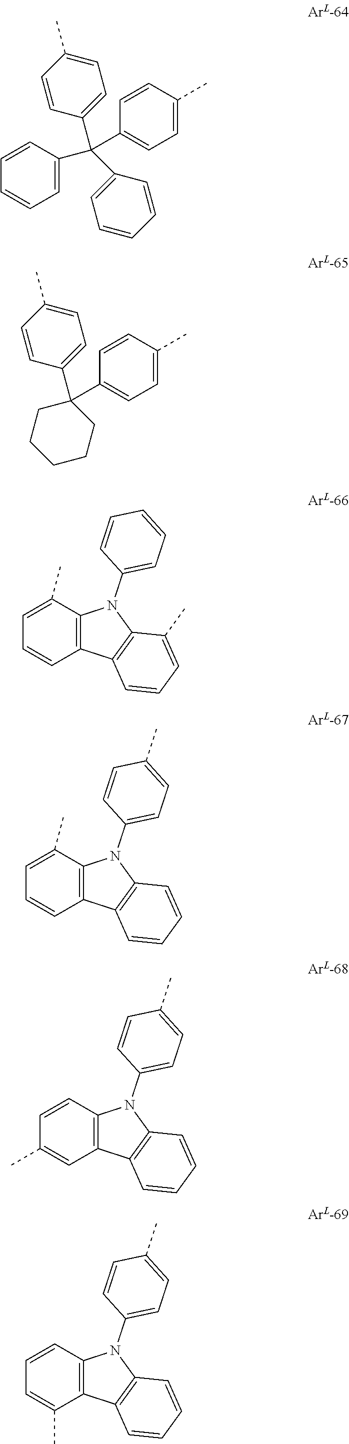

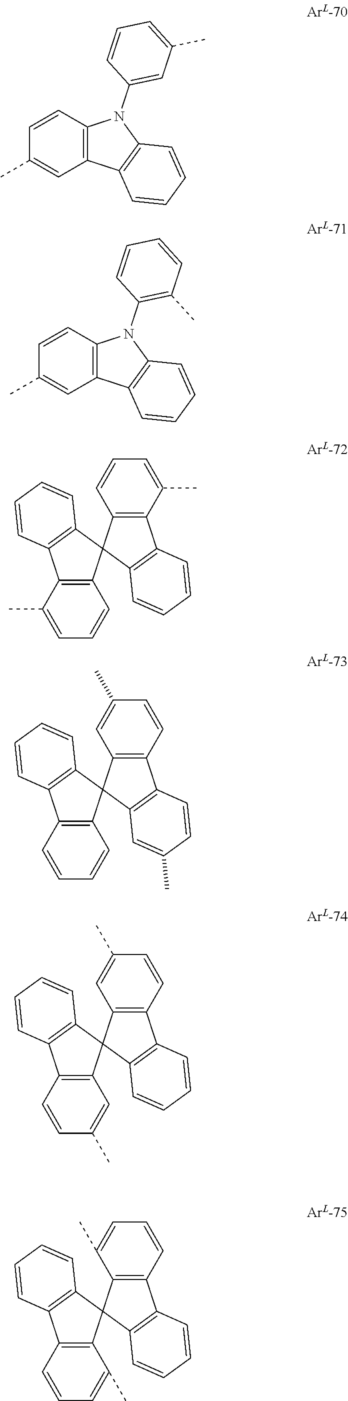

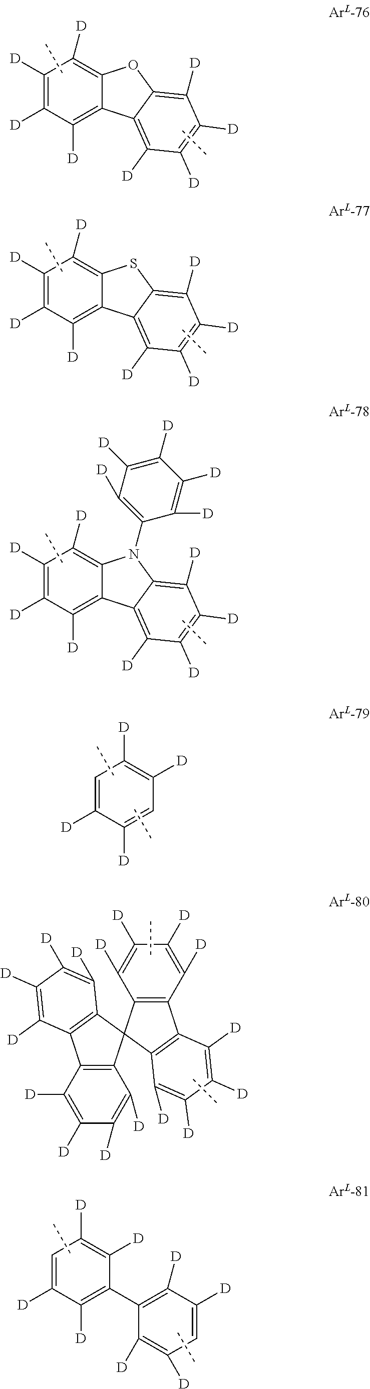

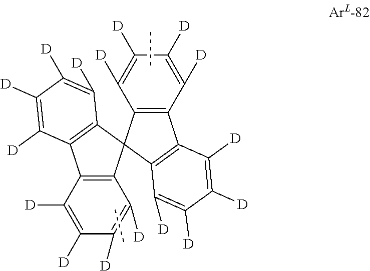

where the dotted lines represent the bonds to the rest of the formula, and where particular preference is given to the formulae Ar^{L}-1, Ar^{L}-2 and Ar^{L}-3.

It may further be the case that Ar^{L} is the same or different at each instance and is selected from phenyl, biphenyl, naphthyl and fluorenyl, each substituted by R² radicals, preferably phenyl and biphenyl, more preferably phenyl.

Ar¹ is the same or different at each instance and is selected from aromatic ring systems which have 6 to 40 aromatic ring atoms and are substituted by R⁴ radicals, and heteroaromatic ring systems which have 5 to 40 aromatic ring atoms and are substituted by R⁴ radicals. Preferably, Ar¹ is the same or different at each instance and is selected from aromatic ring systems which have 6 to 25 aromatic ring atoms and are substituted by R⁴ radicals, and heteroaromatic ring systems which have 5 to 25 aromatic ring atoms and are substituted by R⁴ radicals.

Preferred Ar¹ groups are the same or different at each instance and are selected from monovalent groups derived from benzene, biphenyl, terphenyl, quaterphenyl, naphthalene, fluorene, especially 9,9′-dimethylfluorene and 9,9′-diphenylfluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where each of the monovalent groups is substituted by R⁴ radicals. The Ar¹ groups are also preferably the same or different at each instance and are selected from combinations of 2 to 4 groups derived from benzene, biphenyl, terphenyl, quaterphenyl, naphthalene, fluorene, especially 9,9′-dimethylfluorene and 9,9′-diphenylfluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where each of the monovalent groups is substituted by R⁴ radicals. More preferably, the Ar¹ groups are the same or different at each instance and are selected from monovalent groups derived from benzene, biphenyl, terphenyl, quaterphenyl, fluorene, benzofluorene, spirobifluorene, more preferably biphenyl, terphenyl, fluorene, spirobifluorene, where each of the monovalent groups is substituted by R⁴ radicals.

Ar¹ is preferably the same or different at each instance and is selected from groups of the following formulae:

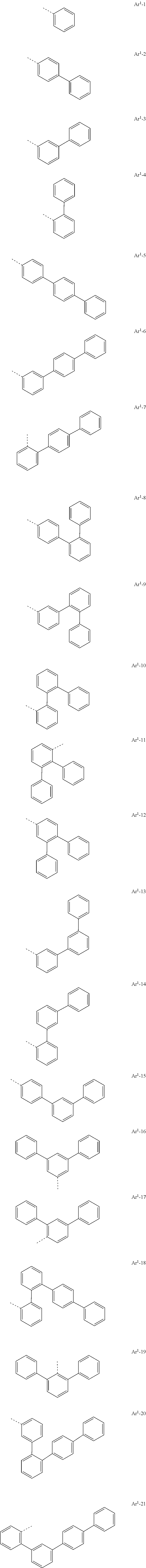

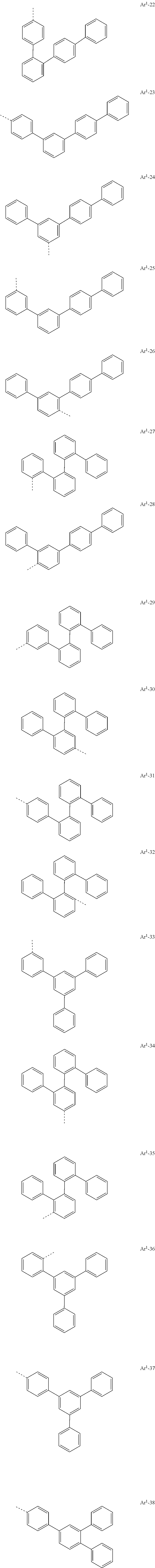

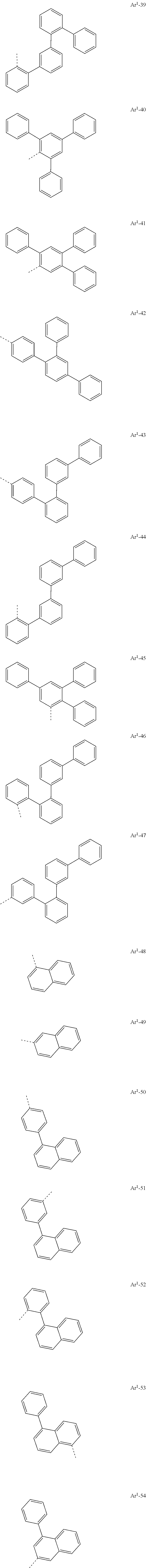

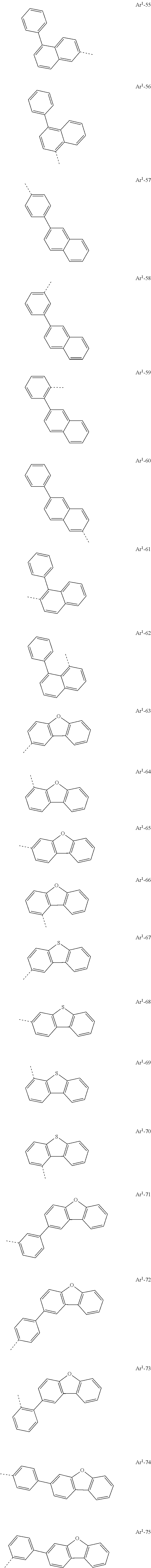

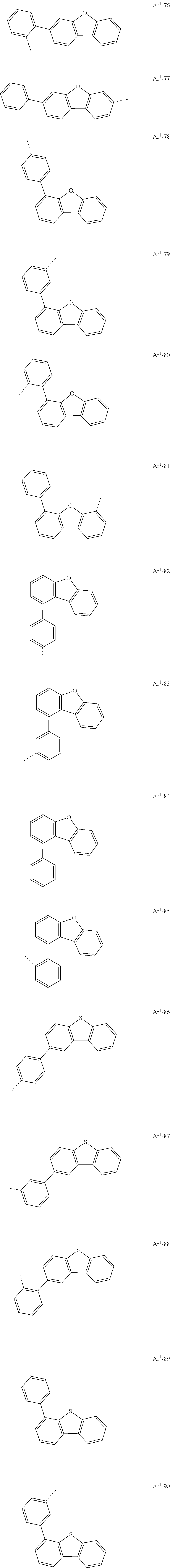

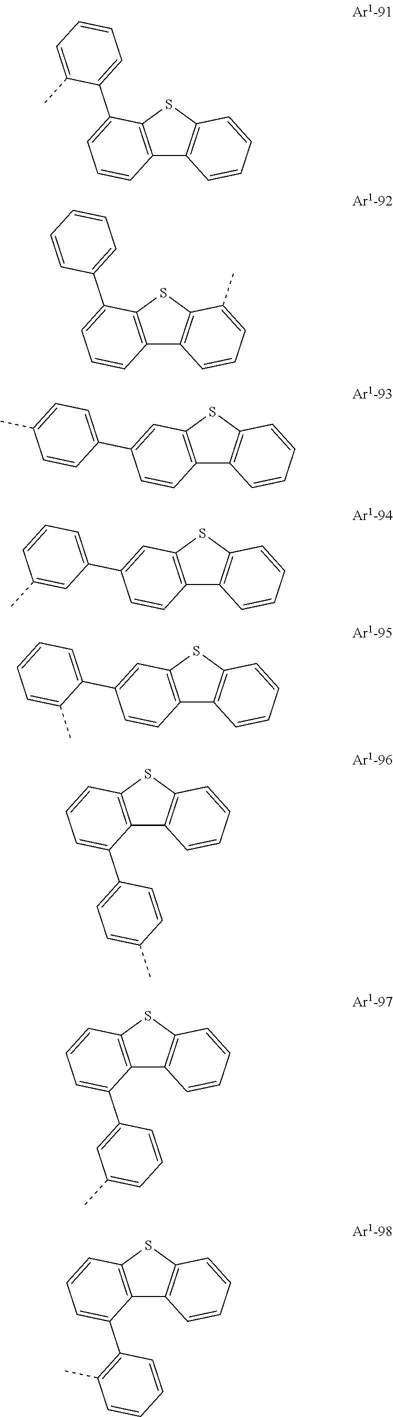

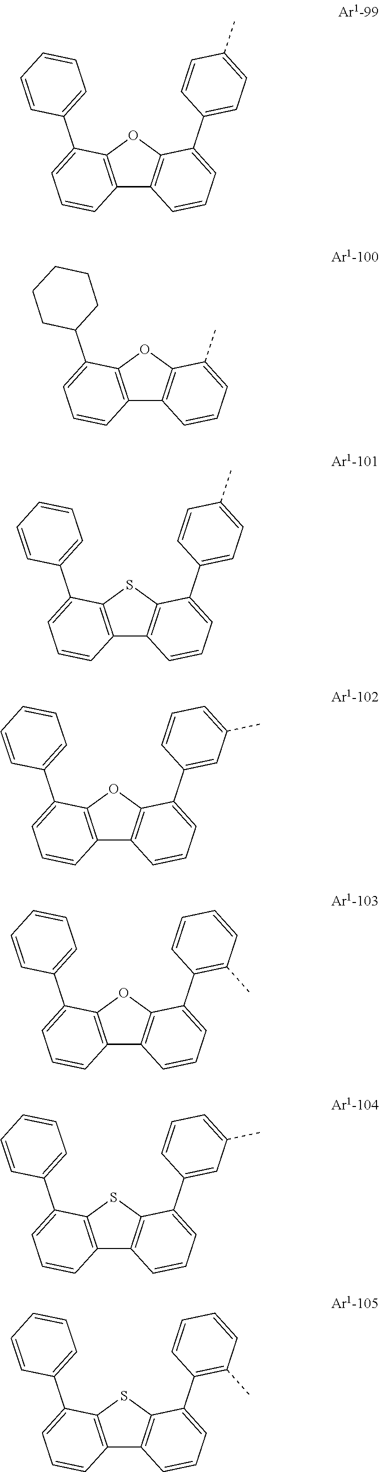

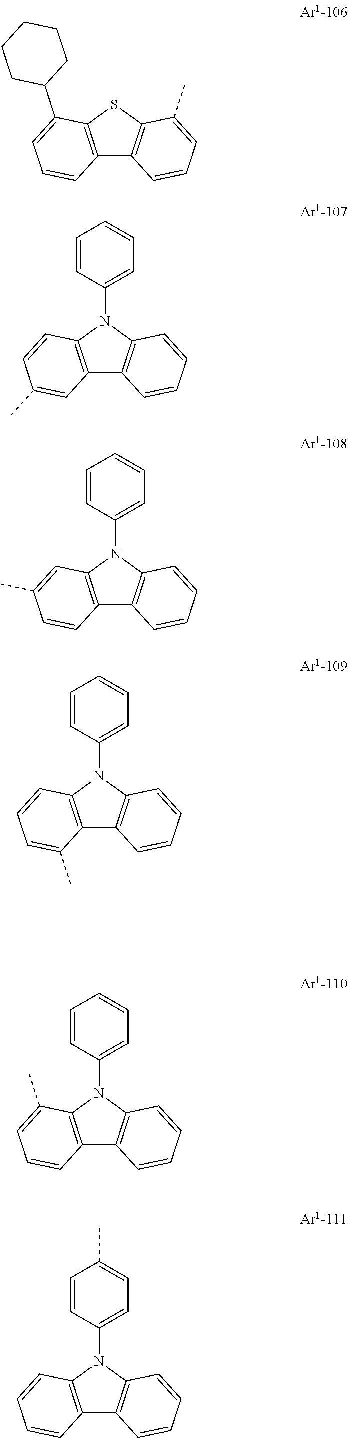

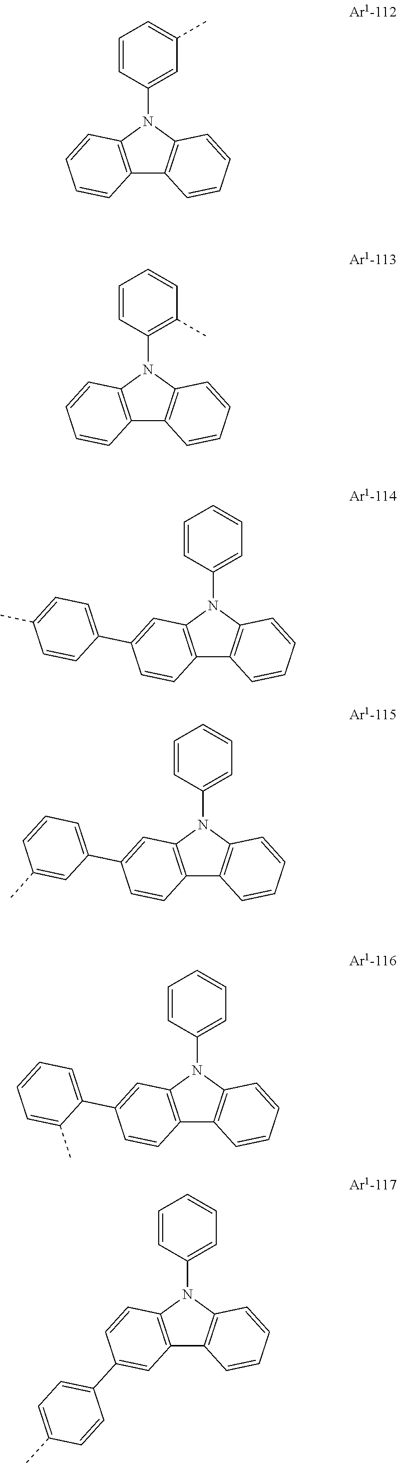

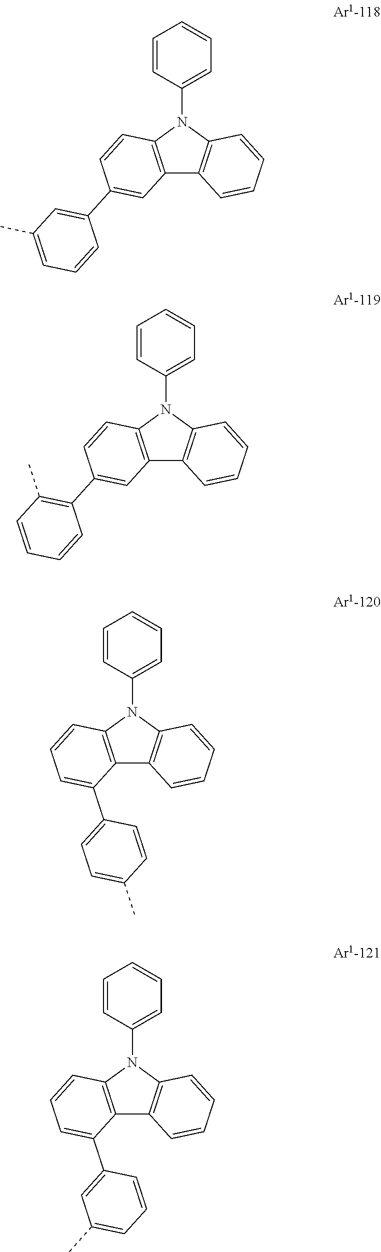

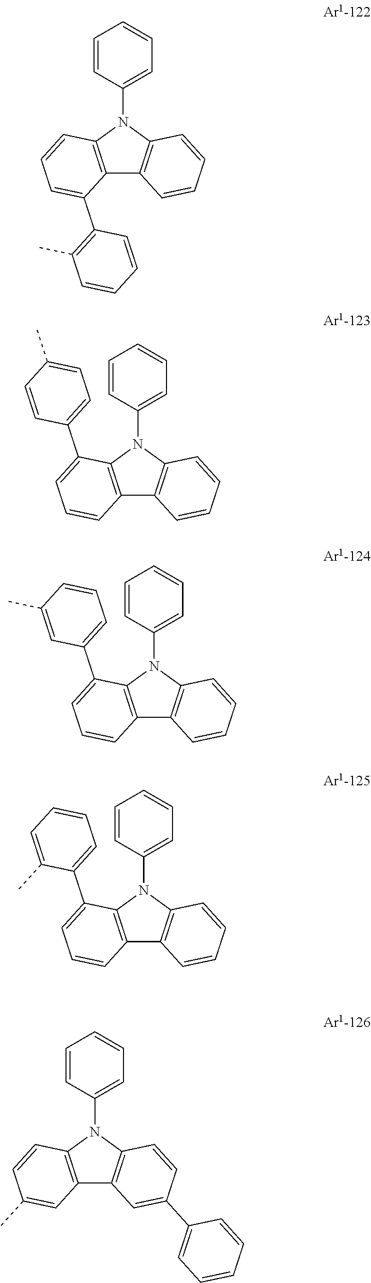

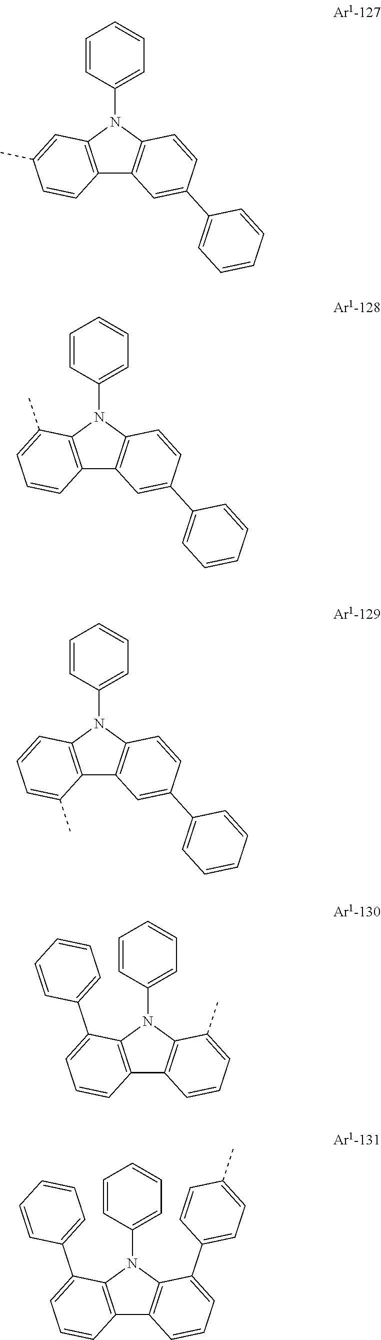

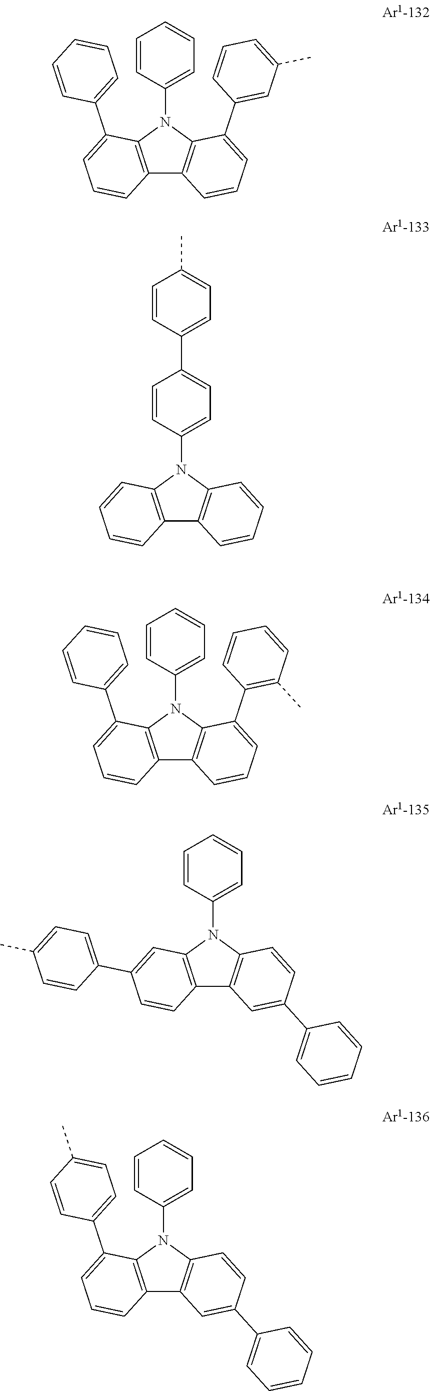

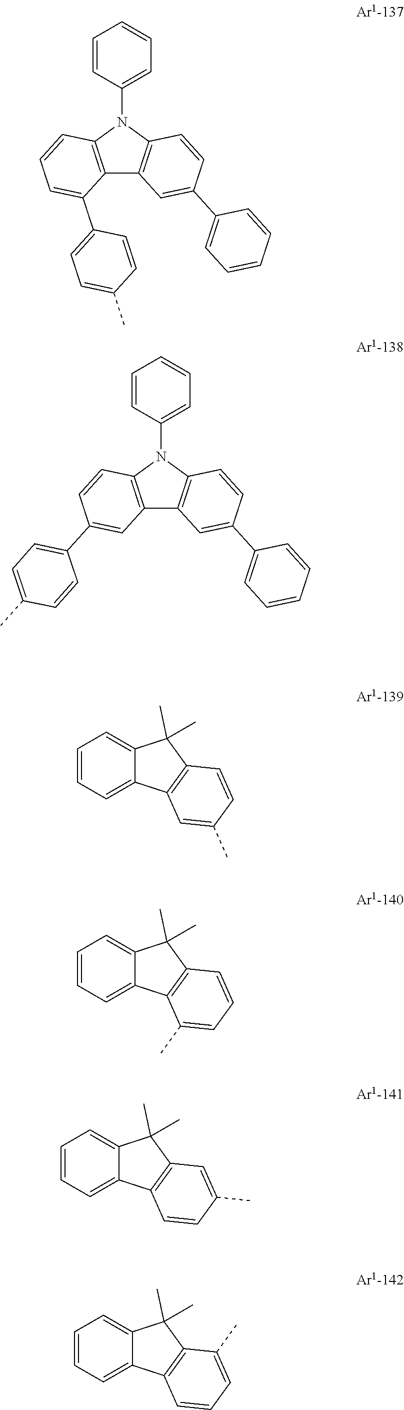

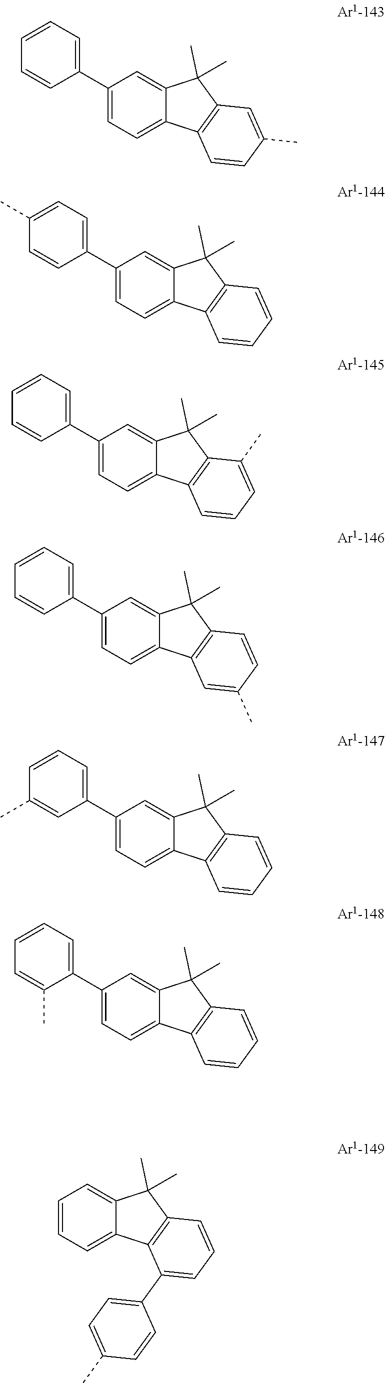

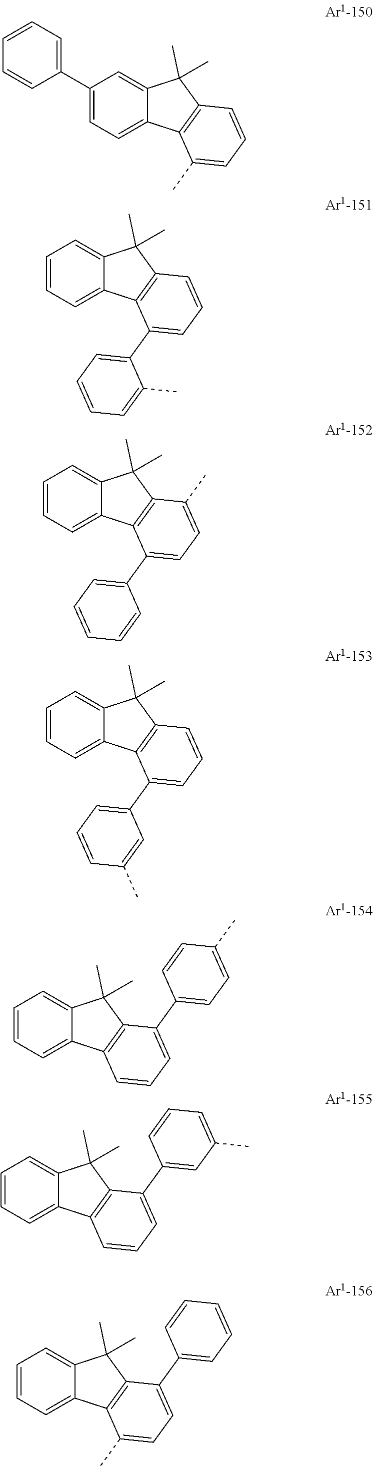

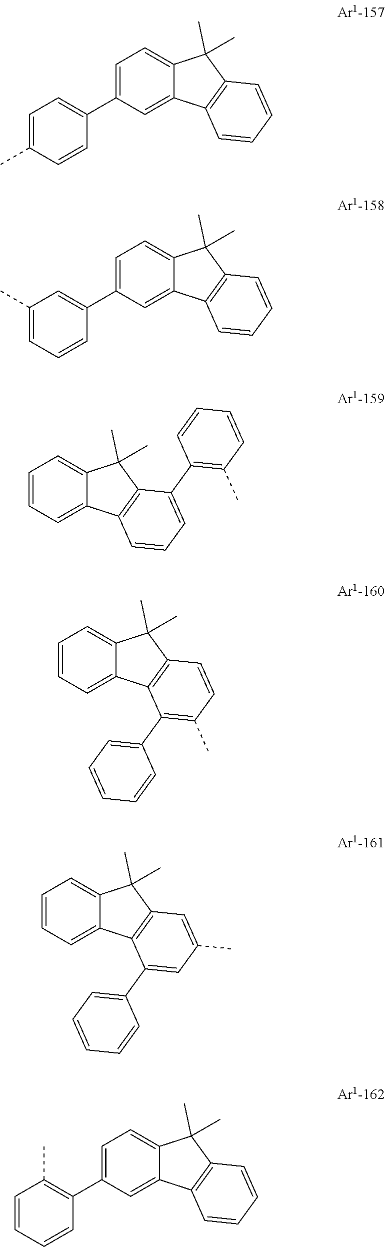

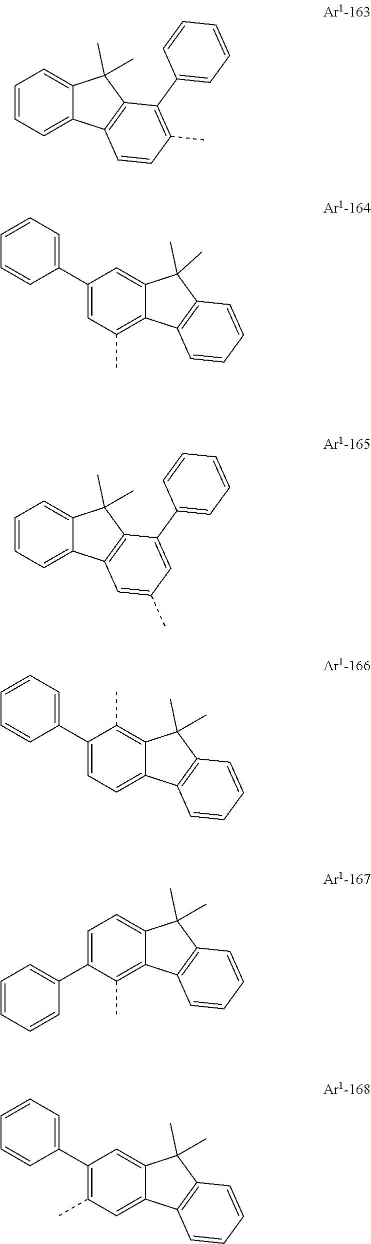

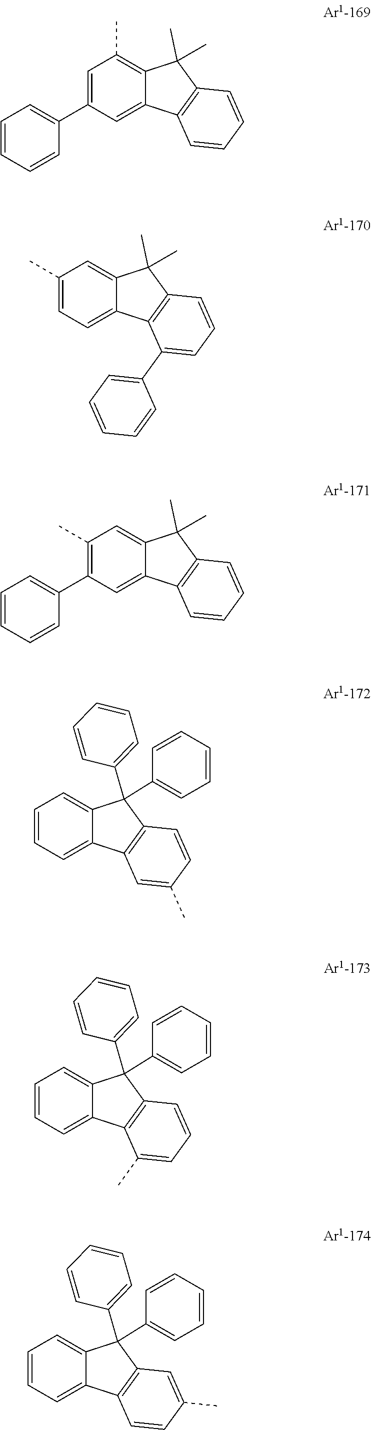

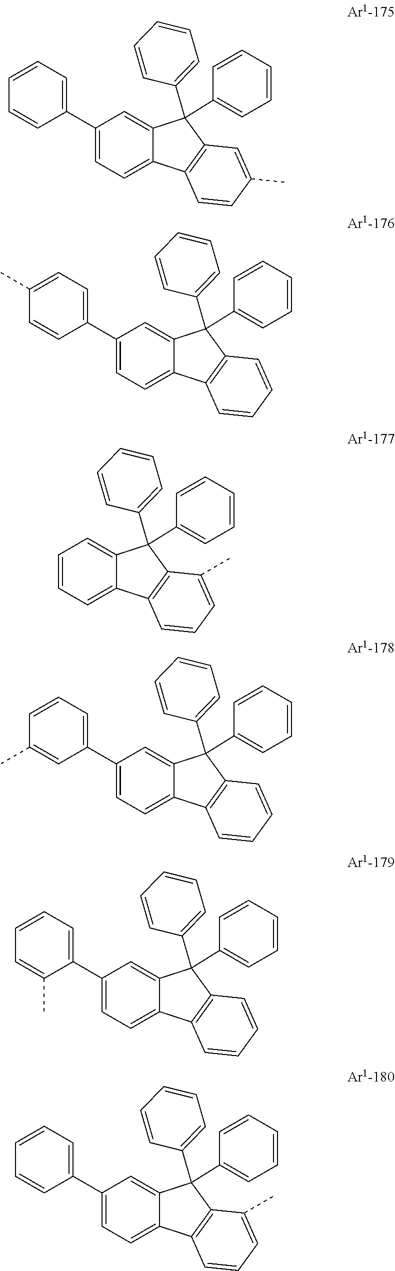

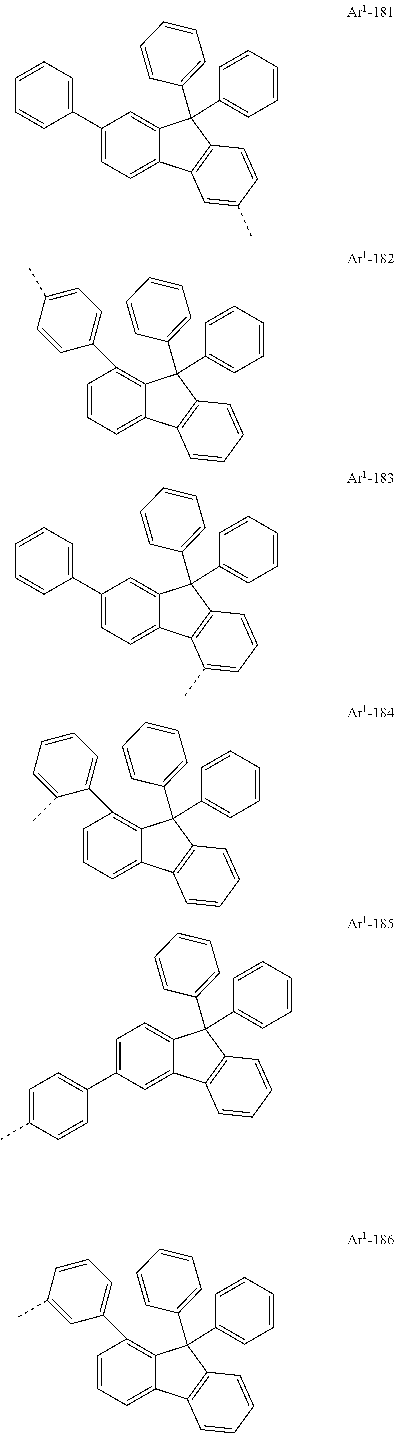

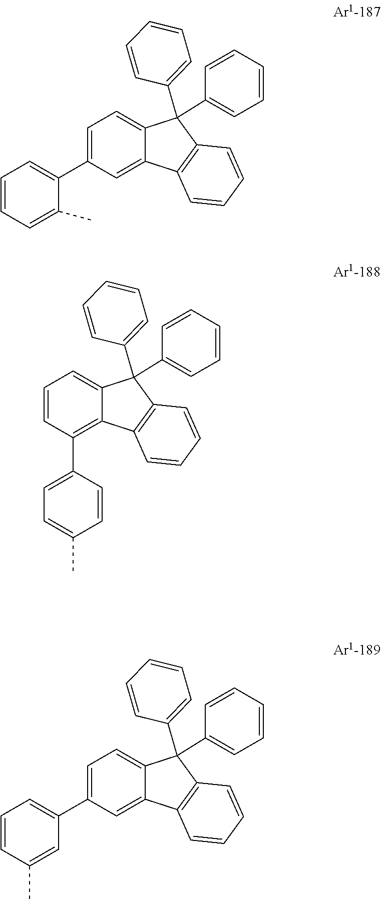

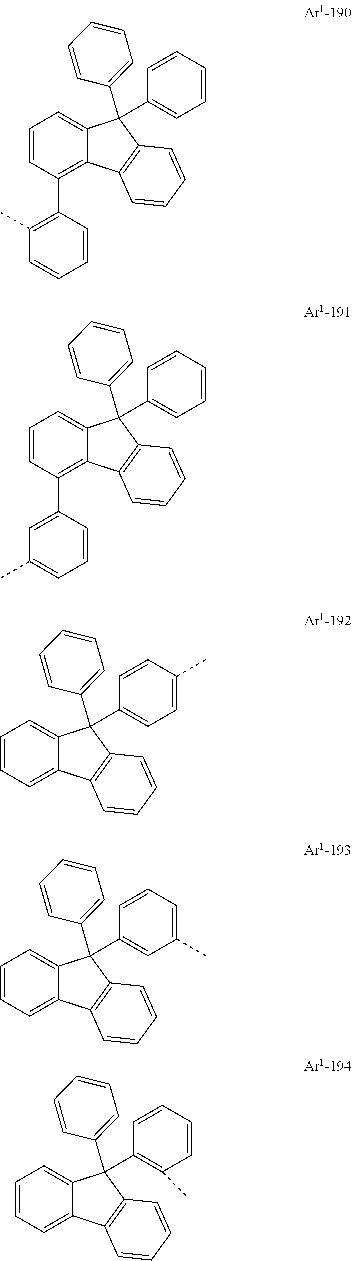

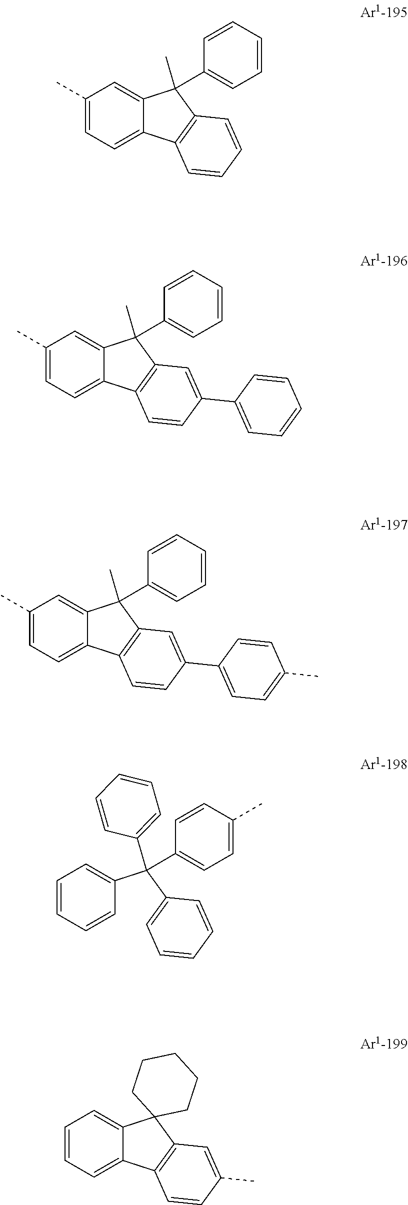

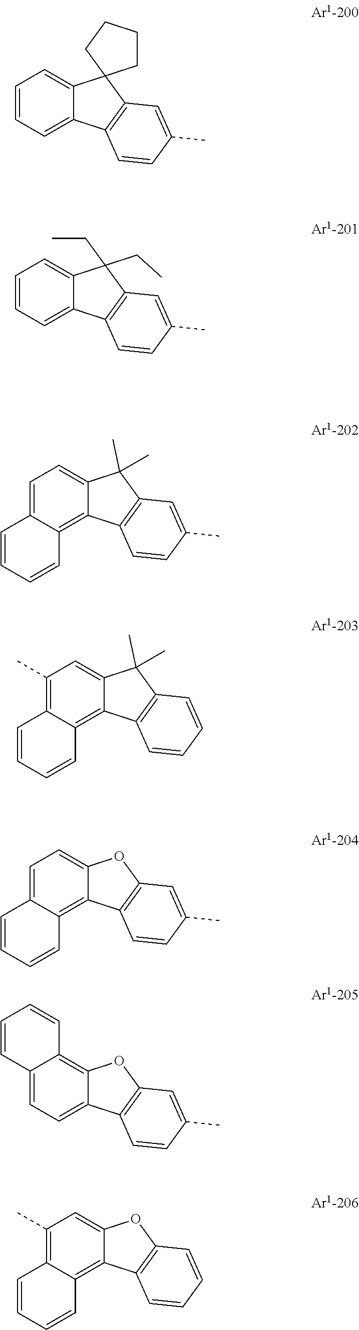

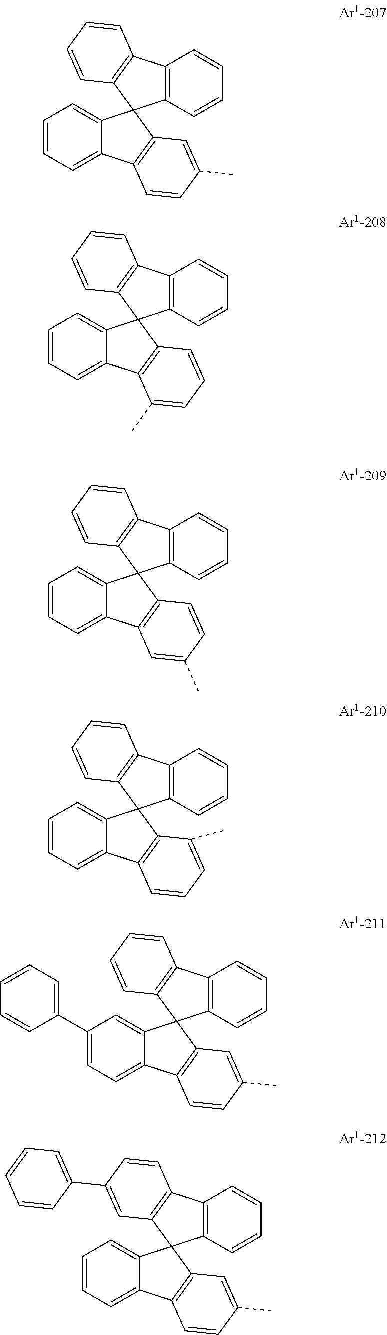

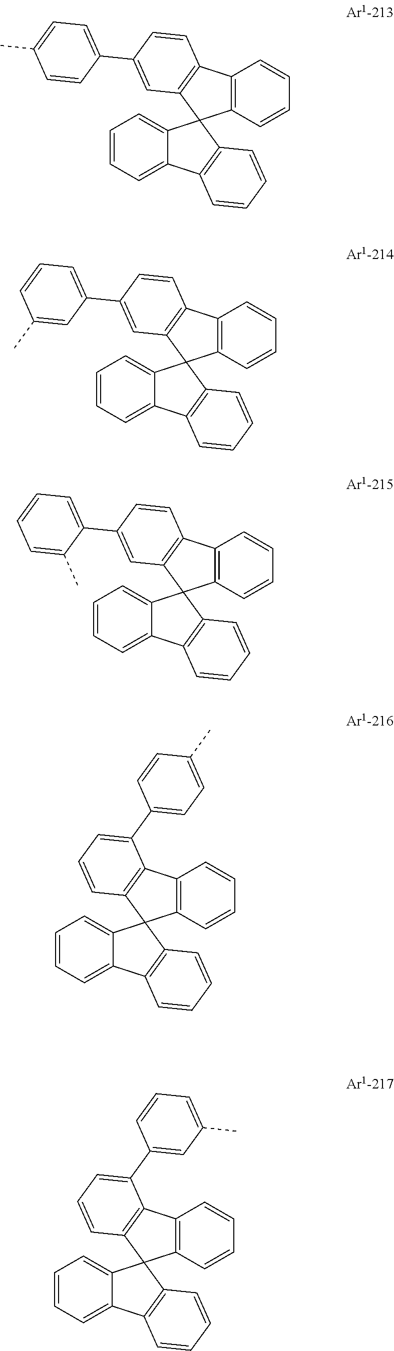

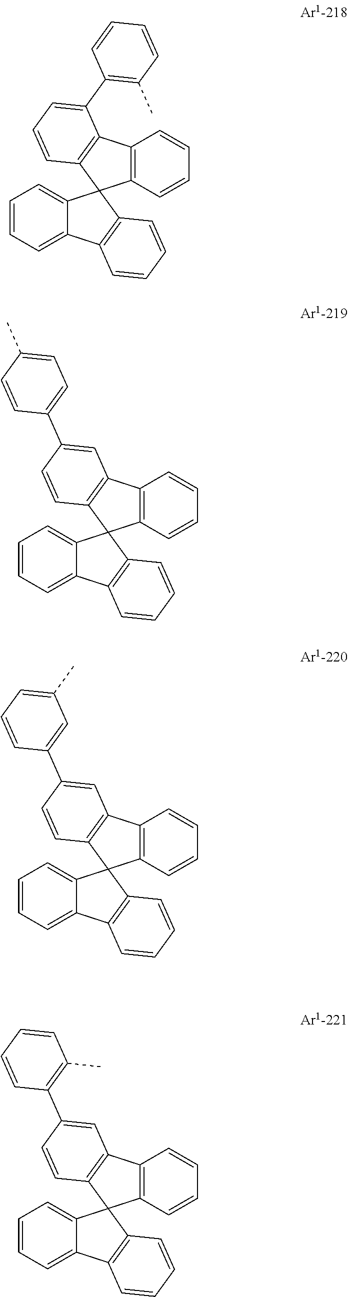

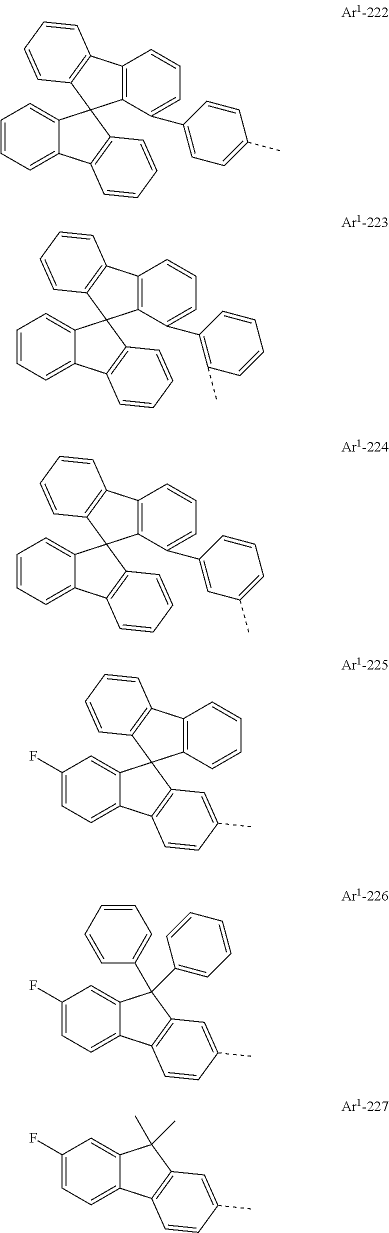

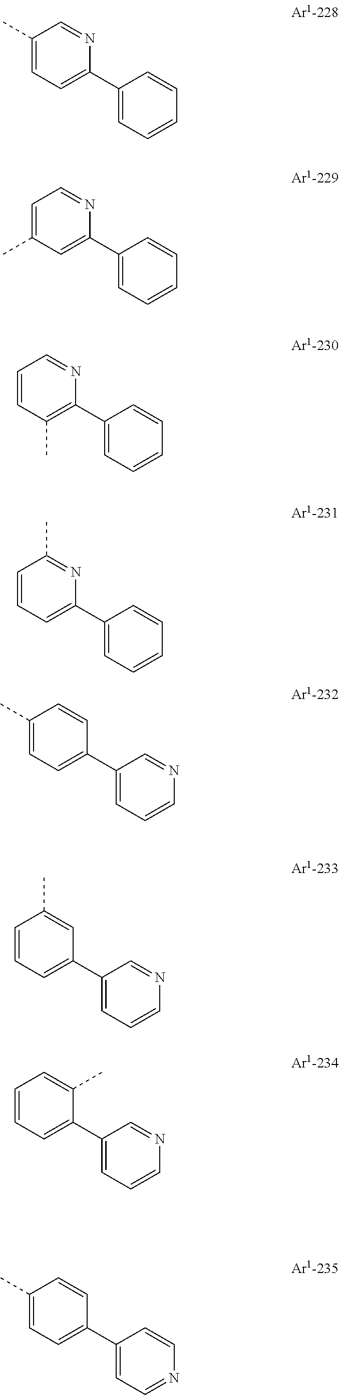

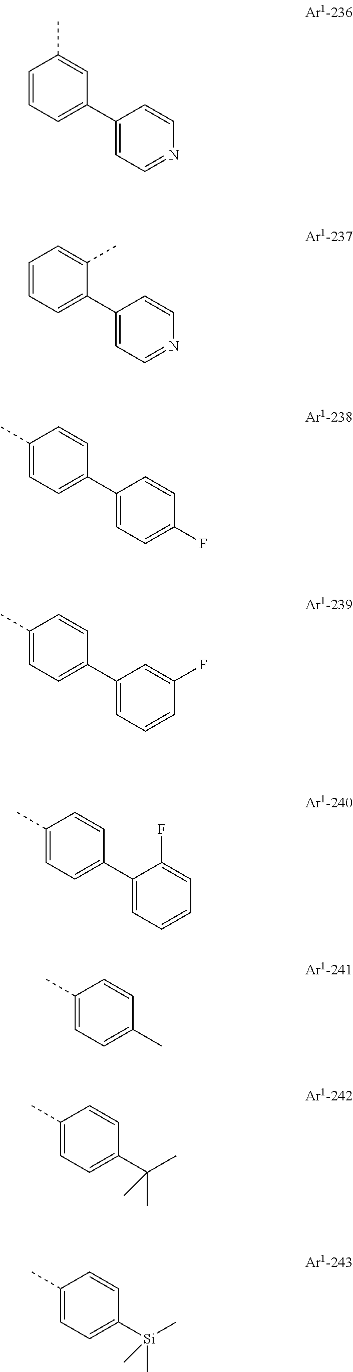

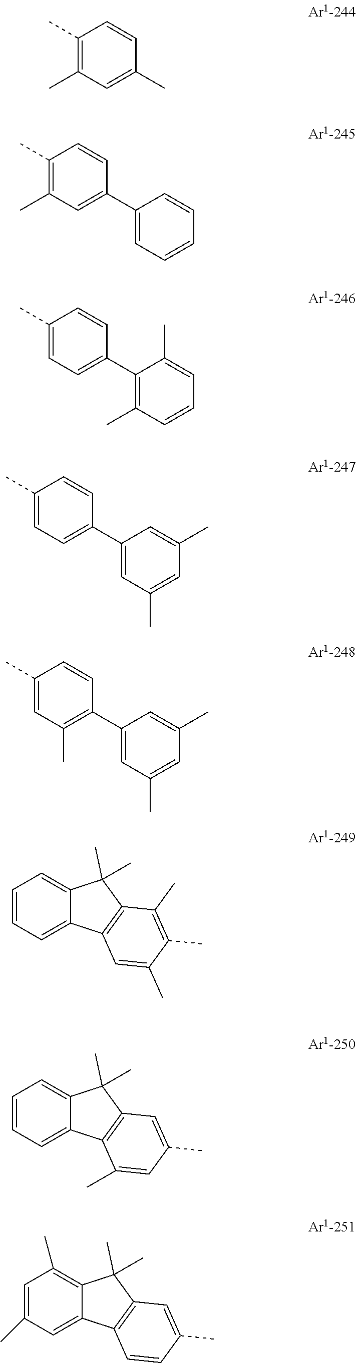

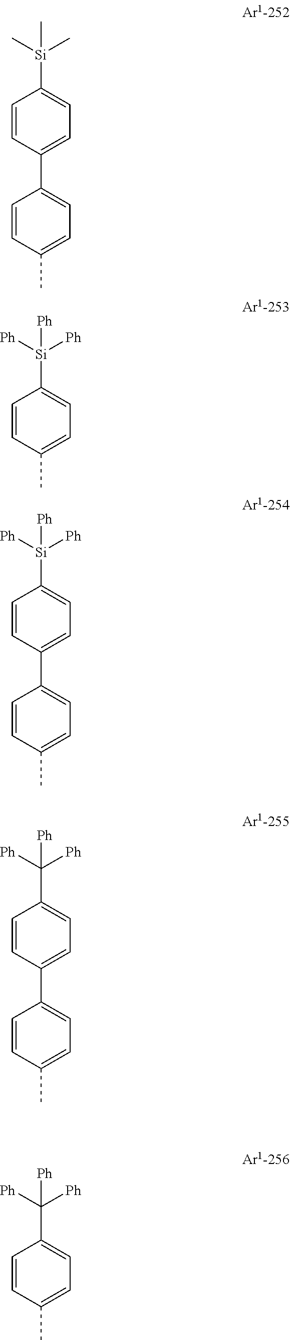

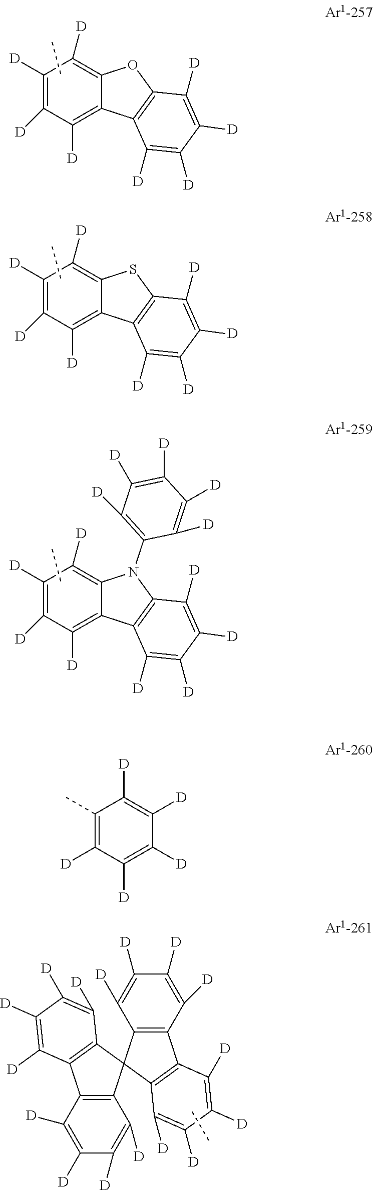

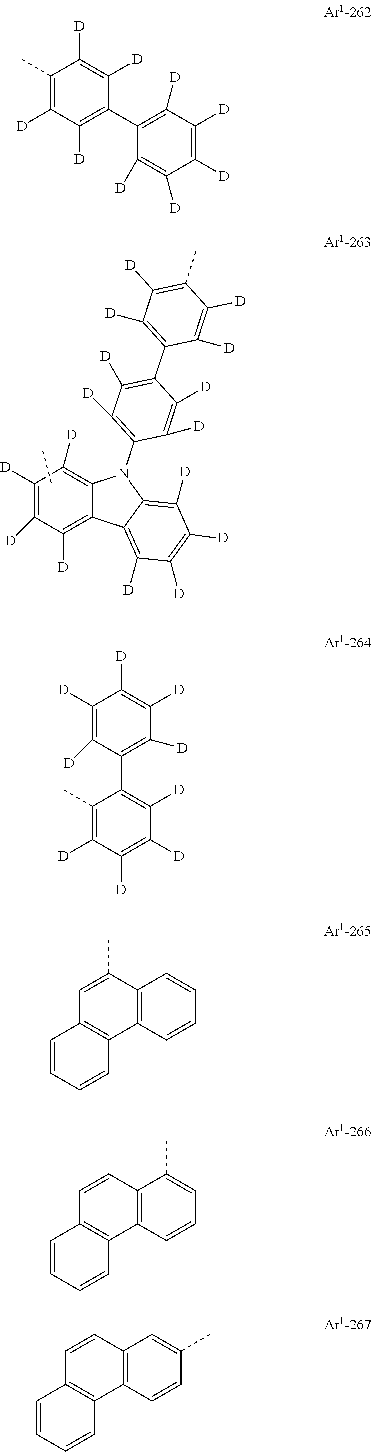

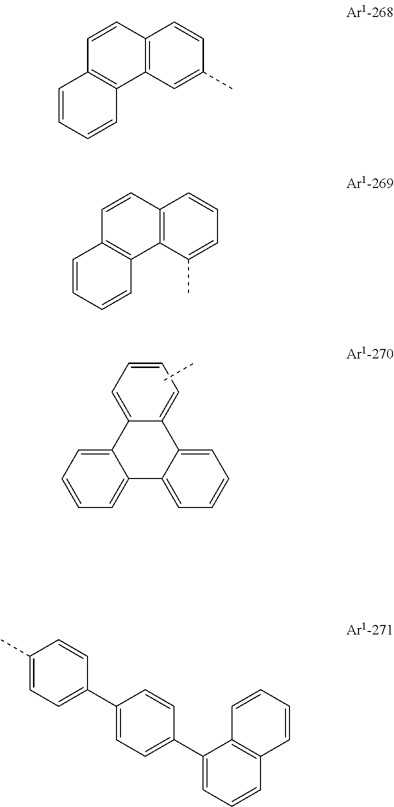

where the dotted line represents the bond to the nitrogen atom and where the groups at the position is shown as unsubstituted may be substituted by R⁴ radicals, and preferably have only H in the positions shown as unsubstituted. Among the abovementioned groups, preference is given to the Ar¹-1 to Ar¹-106 and Ar¹-139 to Ar¹-271 groups, particular preference to the Ar¹-2 to Ar¹-106 and Ar¹-139 to Ar¹-271 groups. It is very particularly preferable when one or both of the Ar¹ groups, preferably both of the Ar¹ groups, are selected from Ar¹-2, Ar¹-5, Ar¹-48, Ar¹-50, Ar¹-74, Ar¹-78, Ar¹-140, Ar¹-141, Ar¹-144, Ar¹-149, Ar¹-193, Ar¹-195, Ar¹-257 to Ar¹-264, Ar¹-265, Ar¹-266 Ar¹-268 and Ar¹-271 groups.

Preferably, both Ar¹ groups are selected from groups of the formulae (Ar¹-1) to (Ar¹-10) and (Ar¹-139) to (Ar¹-171), as defined above.

Among the formulae (Ar¹-1) to (Ar¹-10), preference is given to the formulae (Ar¹-1), (Ar¹-2), (Ar¹-3), (Ar¹-6), (Ar¹-7), (Ar¹-8) and (Ar¹-9).

In an alternative embodiment, it may preferably be the case that one or both of the Ar¹ groups, preferably both of the Ar¹ groups, are selected from the groups Ar¹-2, Ar¹-5, Ar¹-48, Ar¹-50, Ar¹-78, Ar¹-140, Ar¹-141, Ar¹-149, Ar¹-139 to Ar¹-171, Ar¹-193, Ar¹-265, Ar¹-266, Ar¹-268 and Ar¹-271, more preferably Ar¹-2, Ar¹-139 and Ar¹-141.

In a preferred embodiment, the Ar¹ groups do not contain a carbazole group as a substituent R⁴, R⁶ or R⁷.

It may preferably be the case that the two Ar¹ groups are connected solely via the nitrogen atom to which both groups Ar¹ are bonded, but not via substituents R⁴, R⁶ or R⁷ that can form a ring system.

Ar² is the same or different at each instance and is selected from aromatic ring systems which have 6 to 40 aromatic ring atoms and are substituted by R⁵ radicals, and heteroaromatic ring systems which have 5 to 40 aromatic ring atoms and are substituted by R⁵ radicals. Preferably, Ar² is the same or different at each instance and is selected from aromatic ring systems which have 6 to 25 aromatic ring atoms and are substituted by R⁵ radicals, and heteroaromatic ring systems which have 5 to 25 aromatic ring atoms and are substituted by R⁵ radicals.

Preferred Ar² groups are the same or different at each instance and are selected from ortho-divalent groups derived from benzene, biphenyl, terphenyl, quaterphenyl, naphthalene, fluorene, especially 9,9′-dimethylfluorene and 9,9′-diphenylfluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where each of the divalent groups is substituted by R⁵ radicals. Preferably, in addition, the Ar² groups are the same or different at each instance and are selected from combinations of 2 to 4 groups derived from benzene, biphenyl, terphenyl, quaterphenyl, naphthalene, fluorene, especially 9,9′-dimethylfluorene and 9,9′-diphenylfluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where each of the groups is substituted by R⁵ radicals. More preferably, the Ar² groups are the same or different at each instance and are selected from ortho-divalent groups derived from benzene, biphenyl, terphenyl, quaterphenyl, fluorene, benzofluorene, spirobifluorene, more preferably biphenyl, terphenyl, fluorene, spirobifluorene, where each of the divalent groups is substituted by R⁵ radicals.

Ar² is preferably the same or different at each instance and is selected from groups of the following formulae:

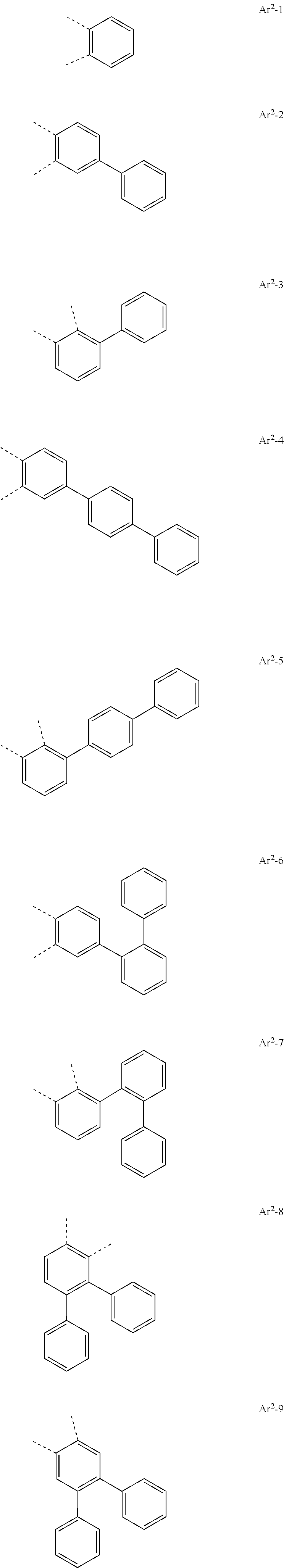

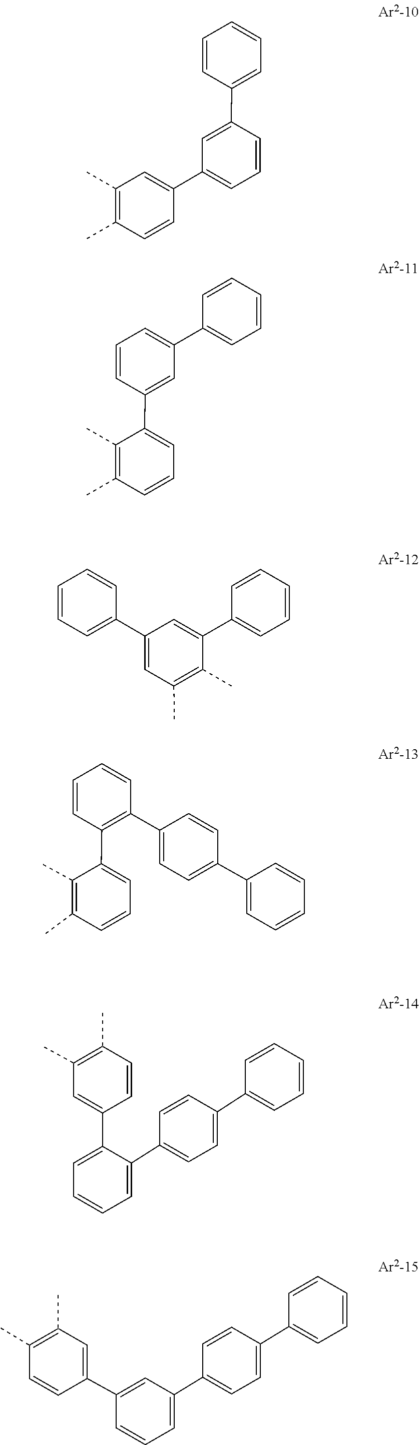

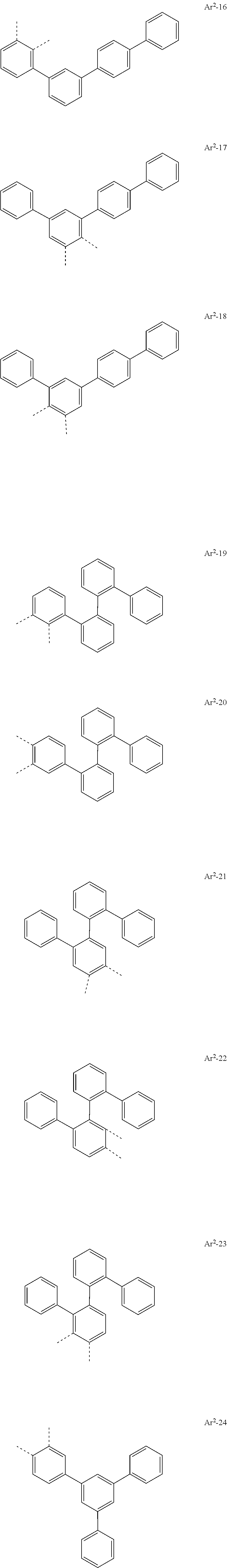

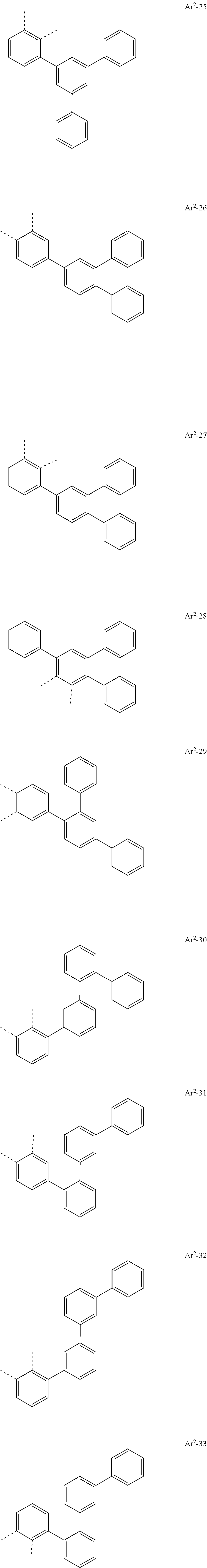

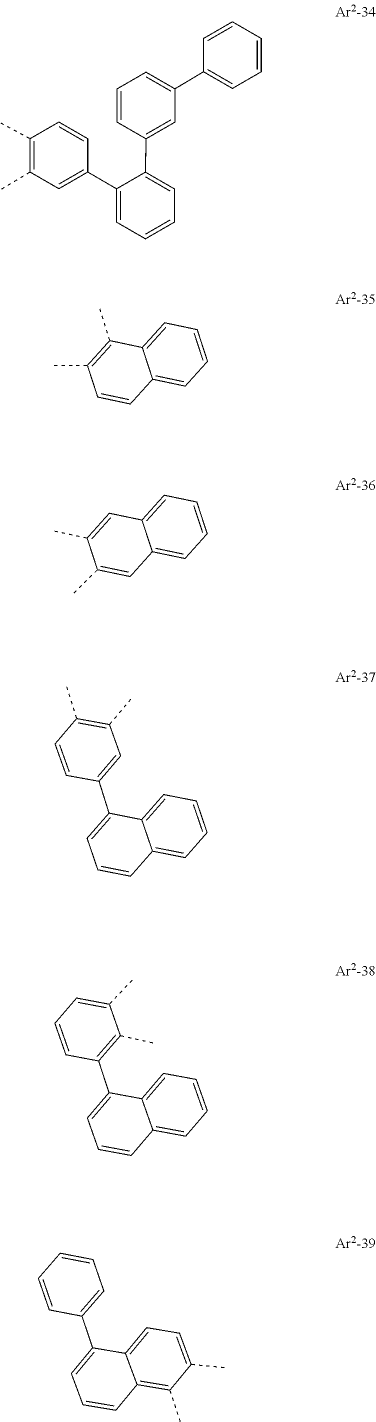

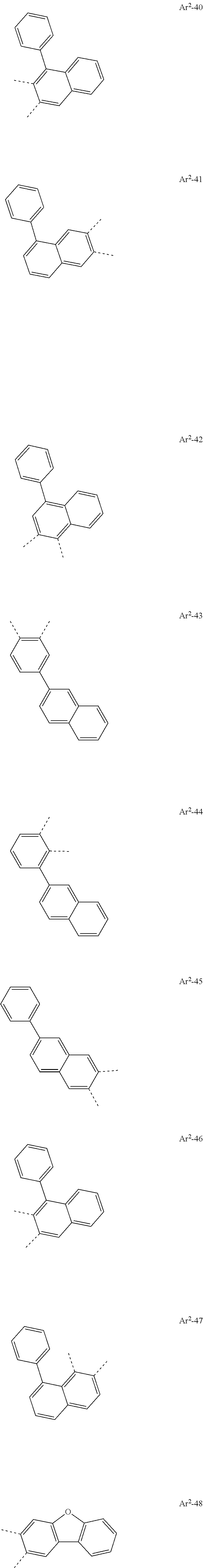

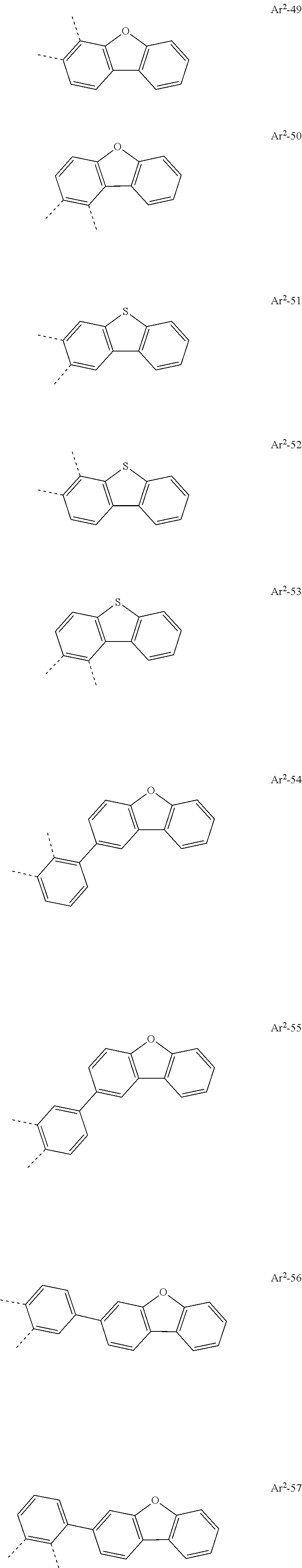

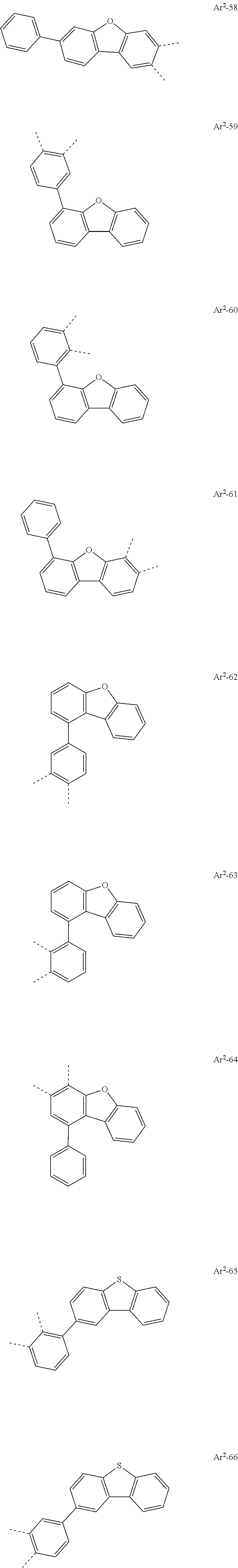

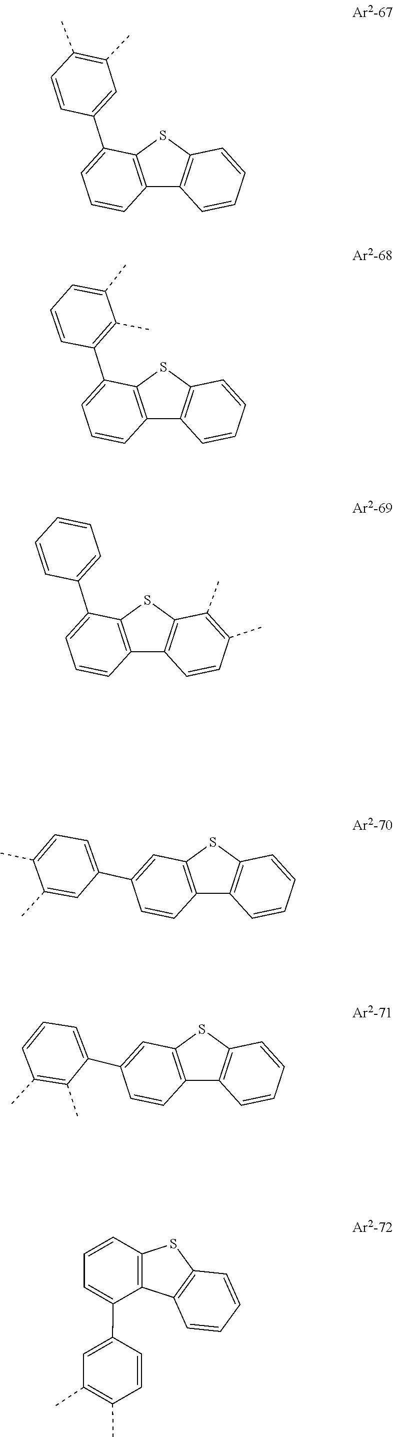

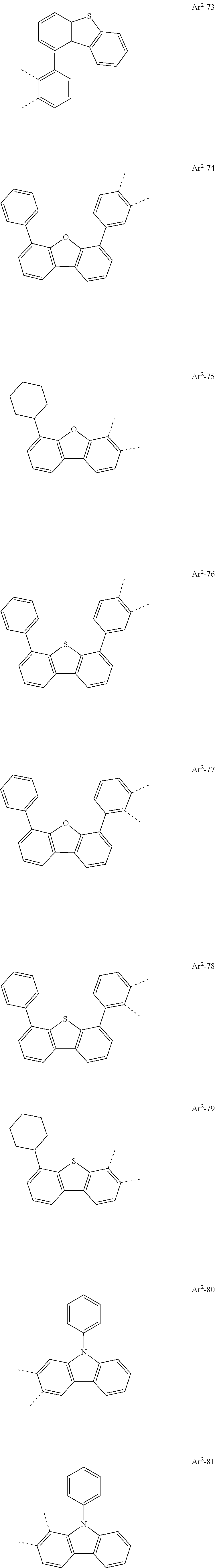

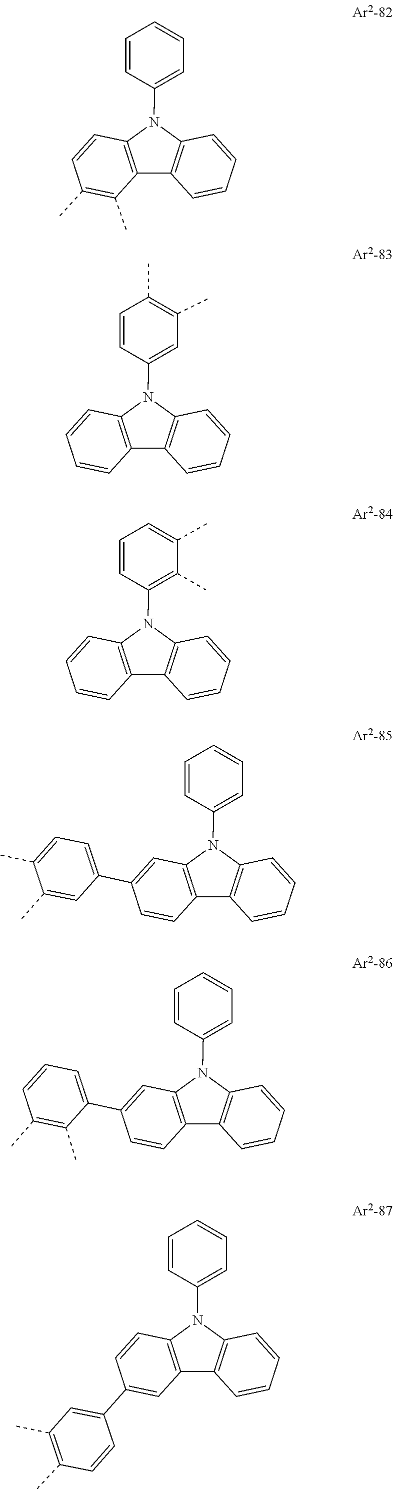

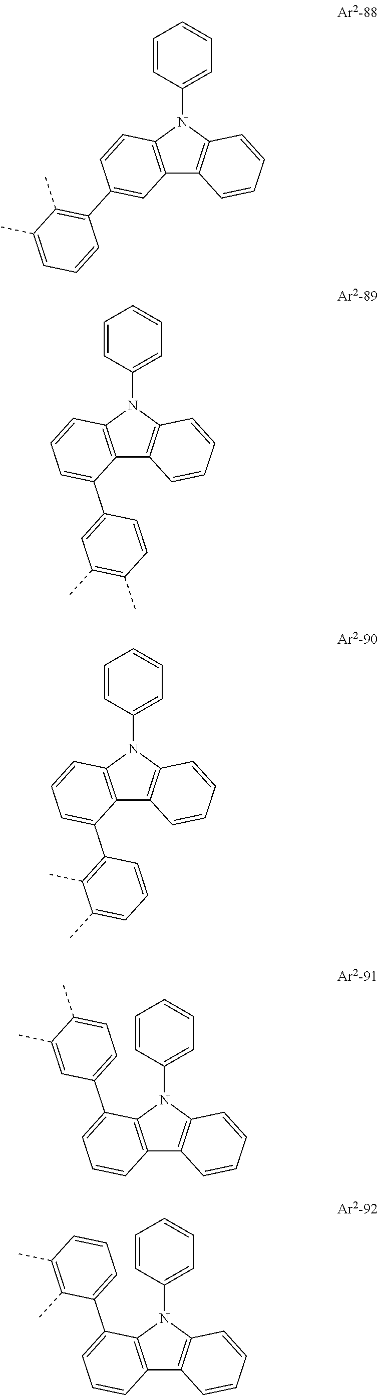

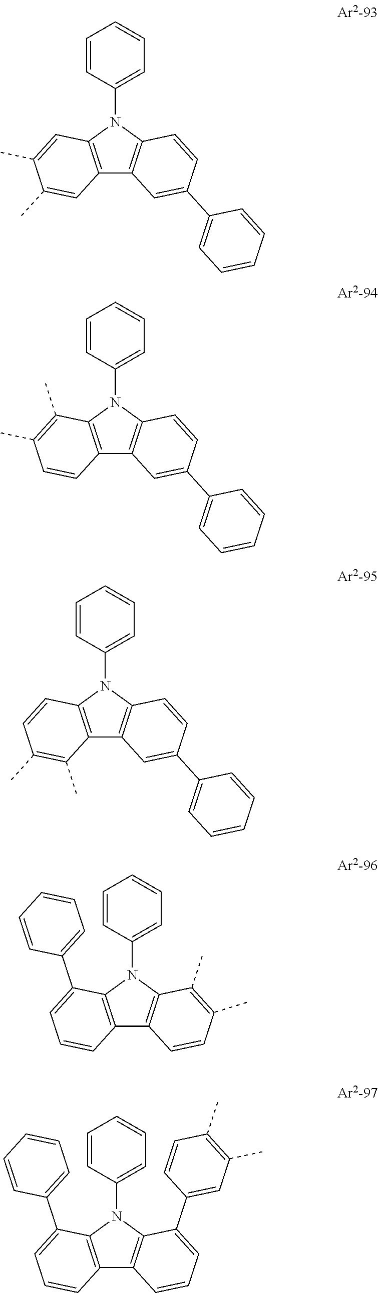

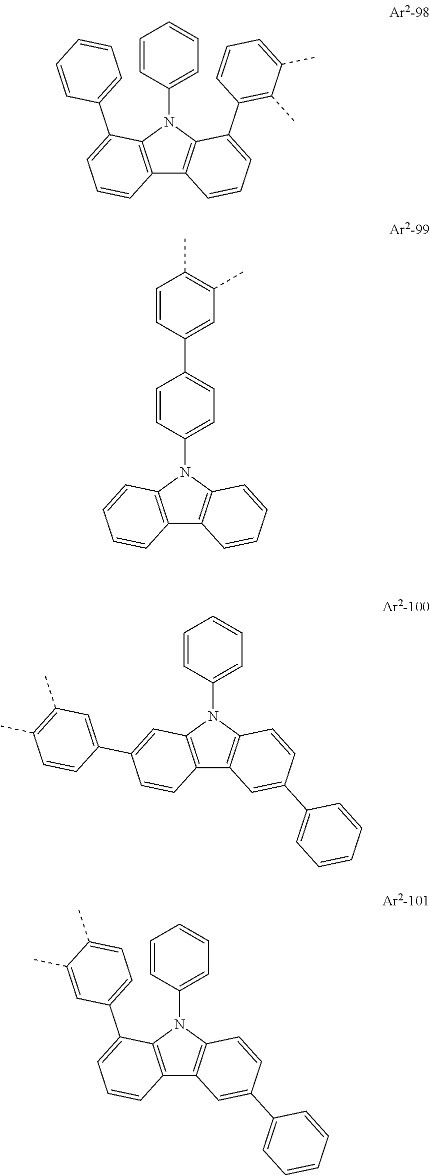

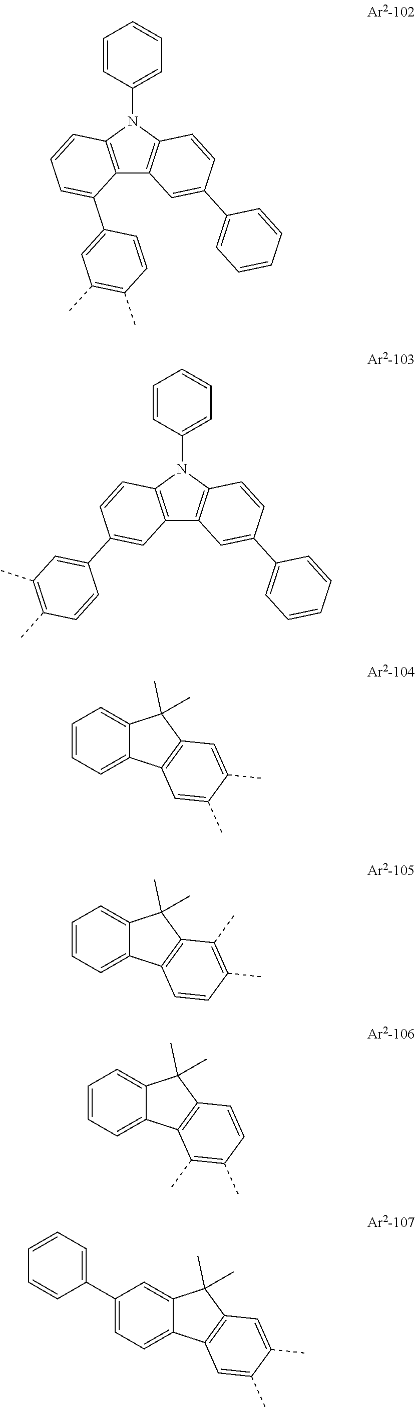

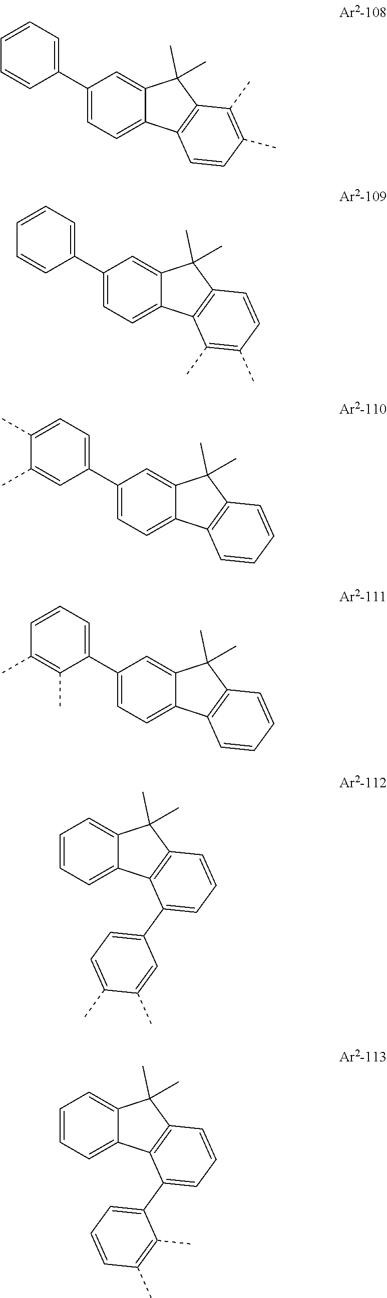

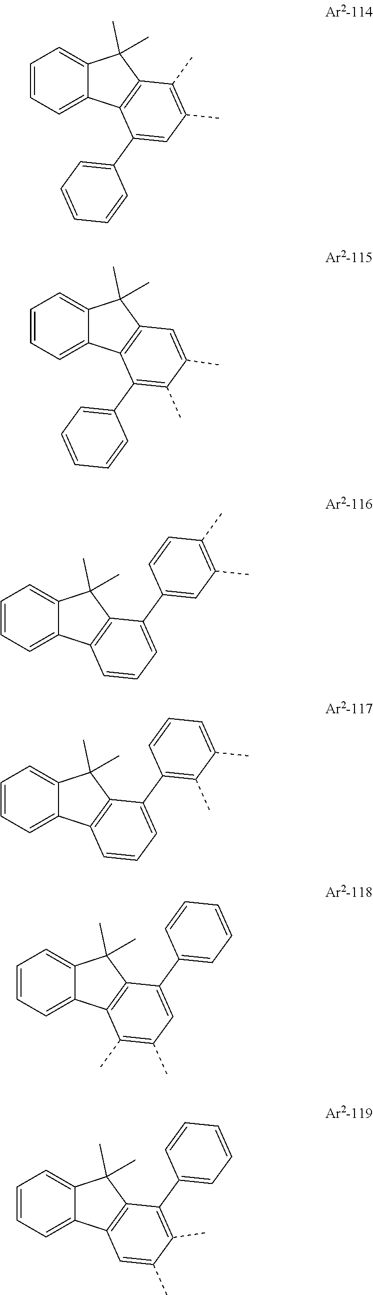

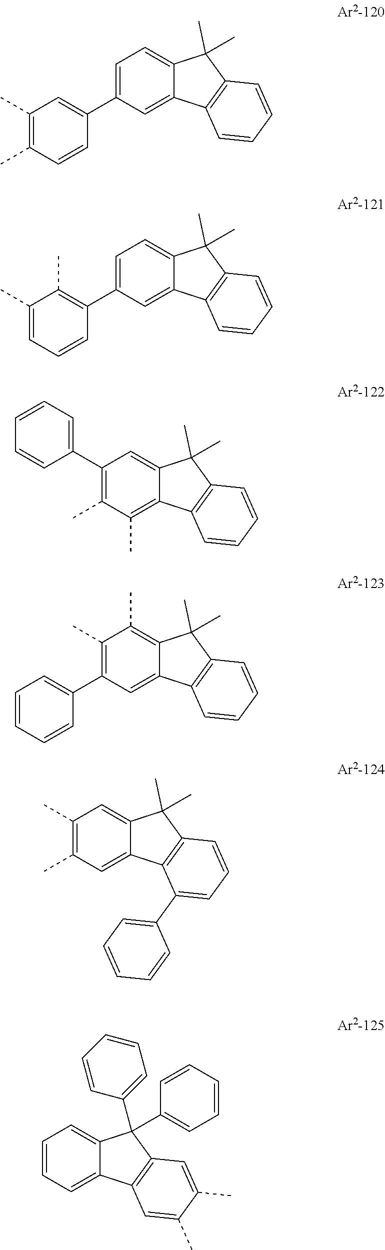

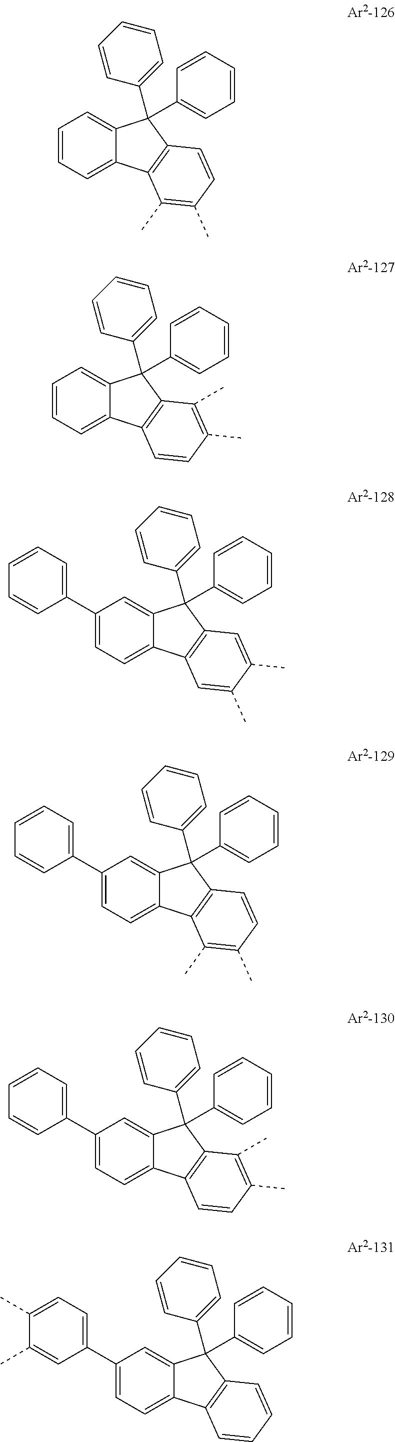

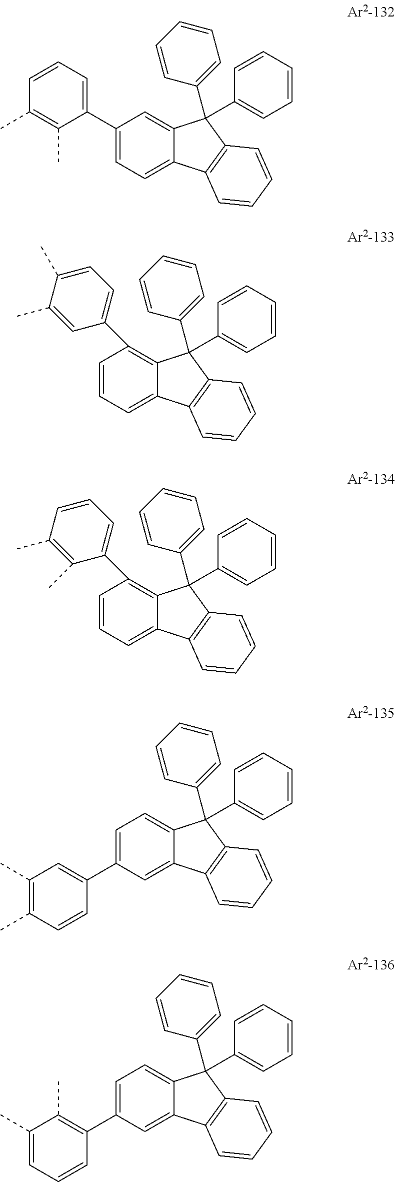

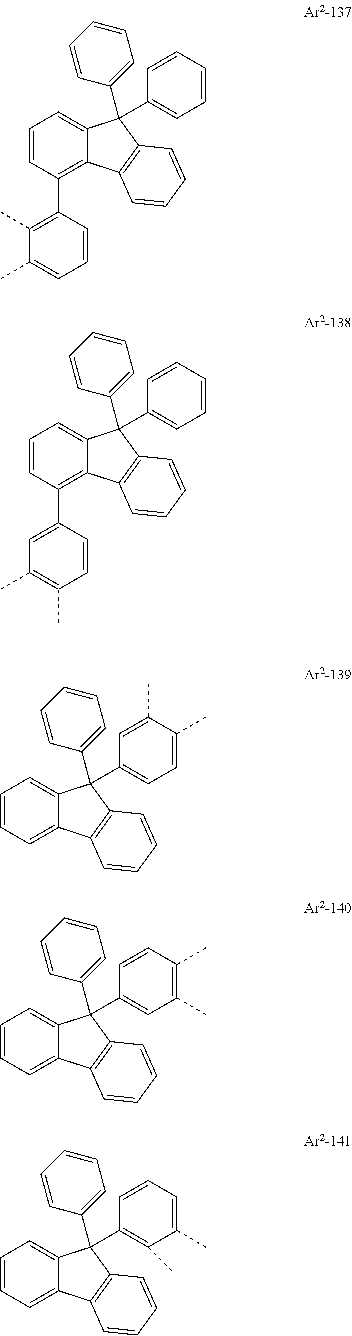

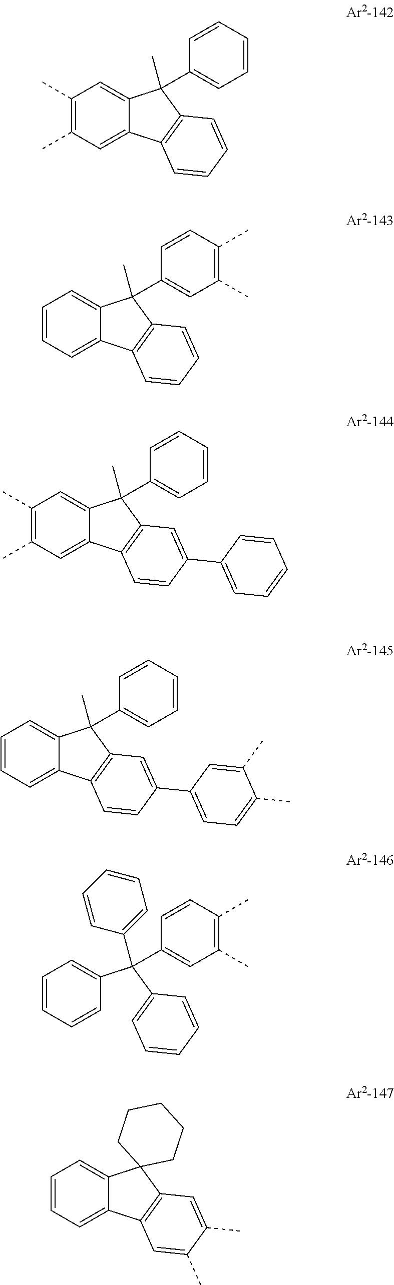

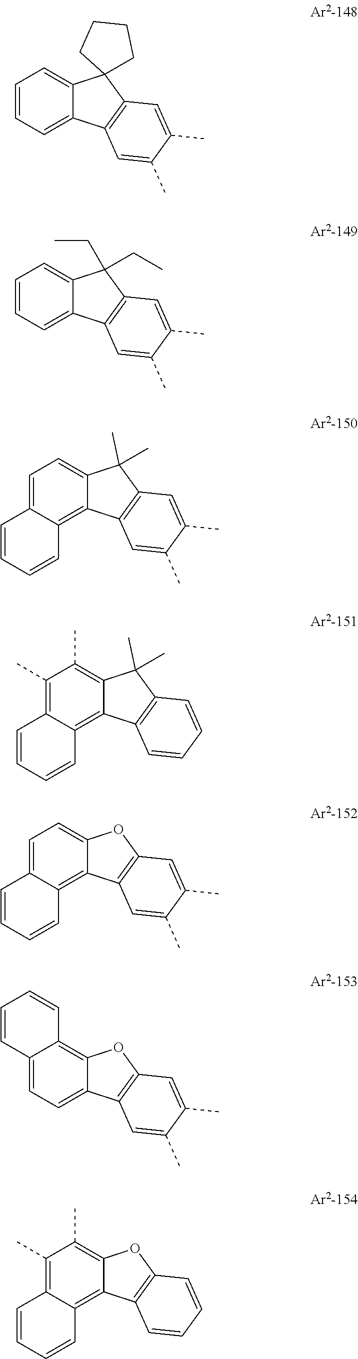

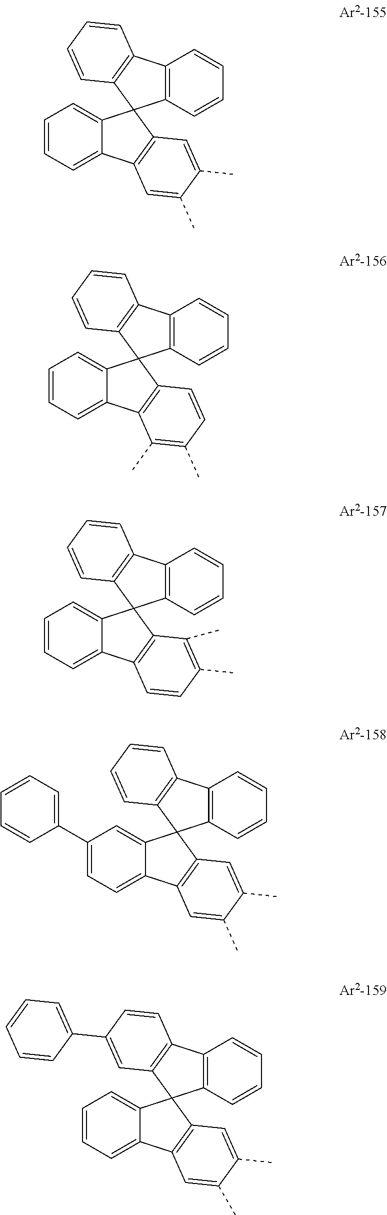

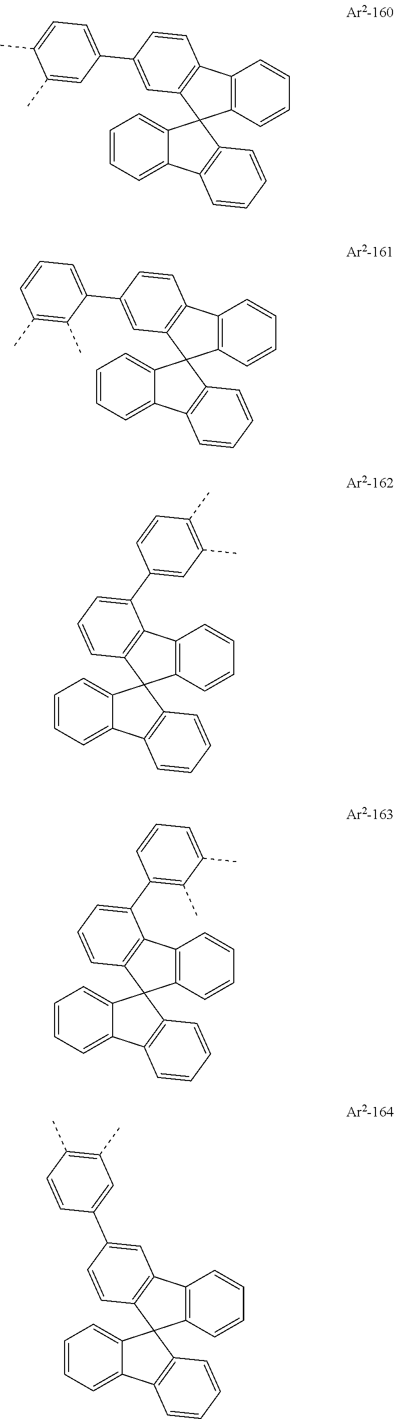

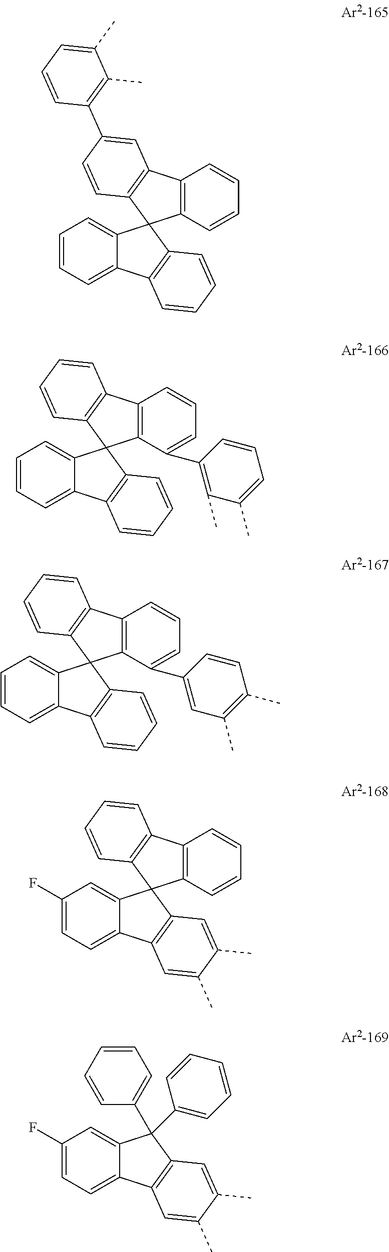

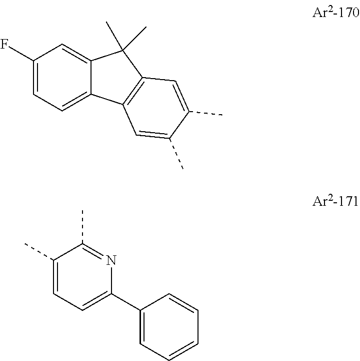

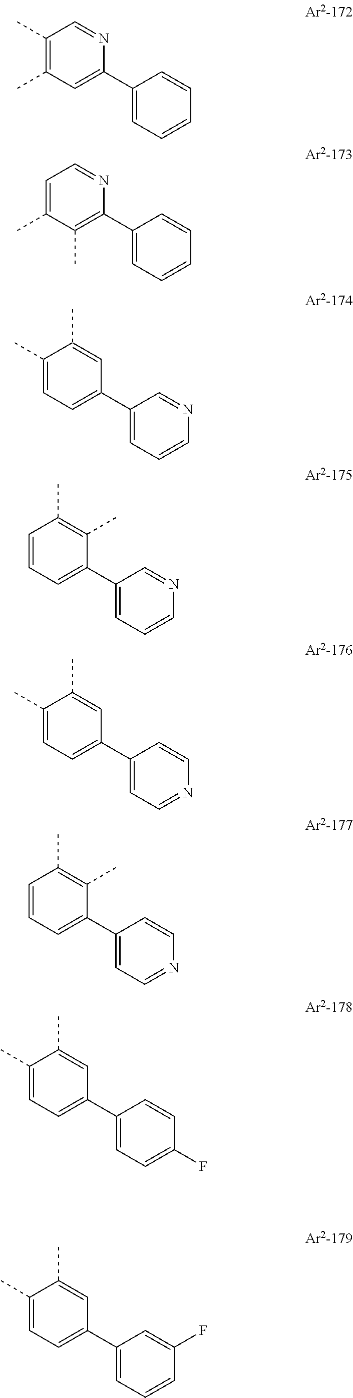

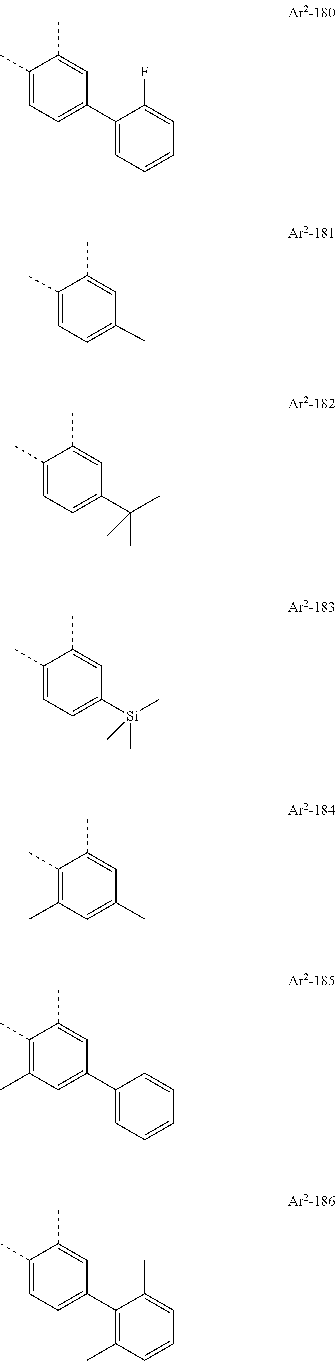

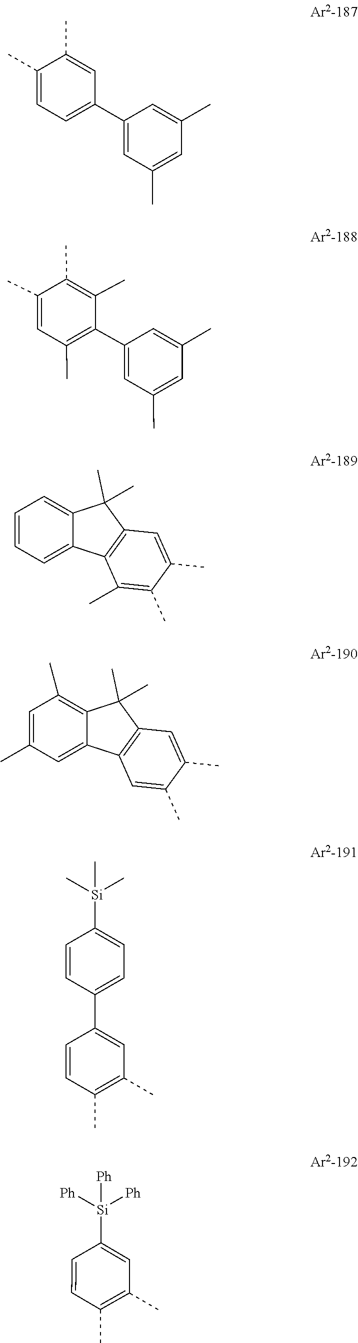

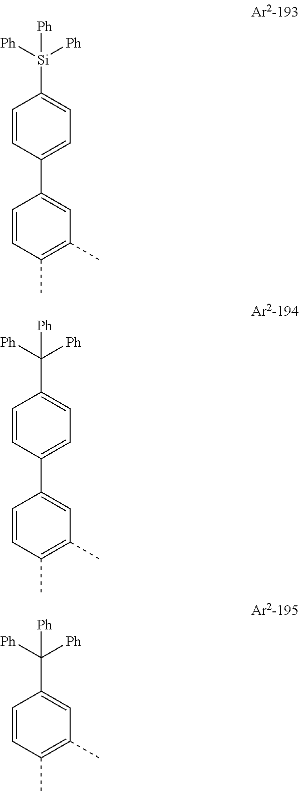

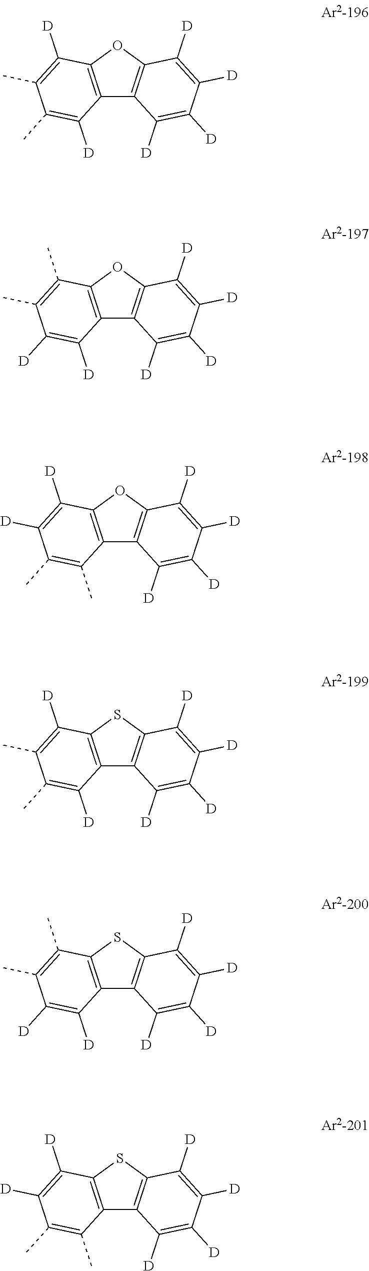

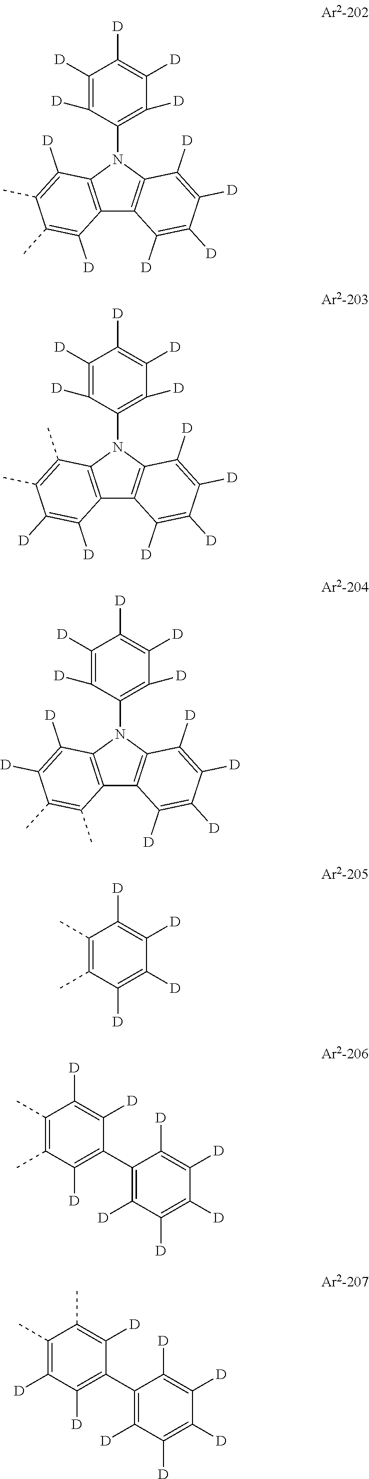

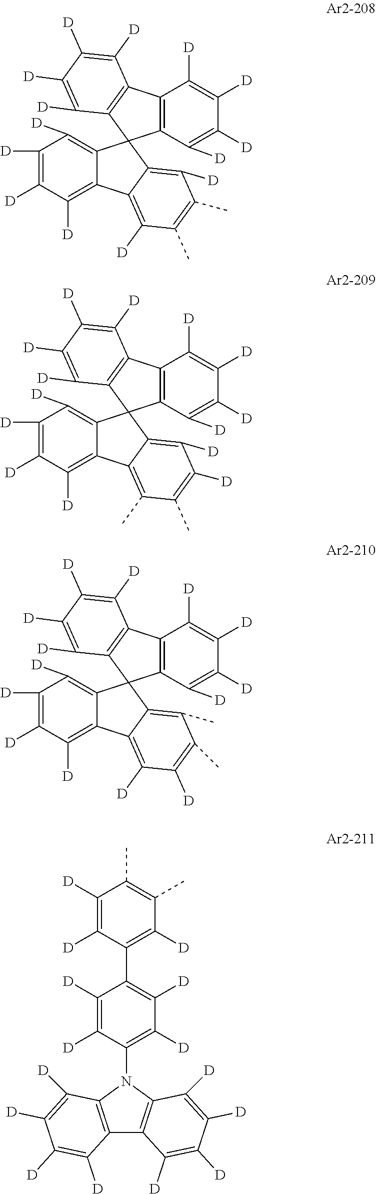

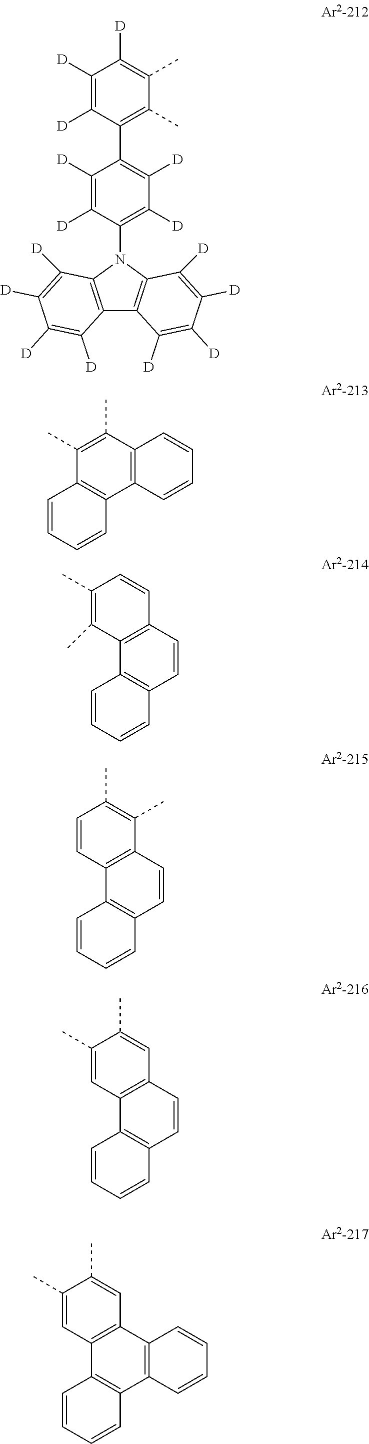

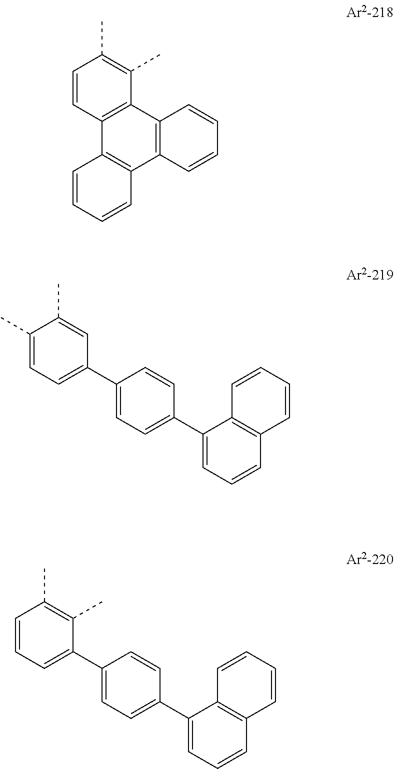

where the dotted line represents the bond to the nitrogen atom or the bond to the V group and where the groups at the positions shown as unsubstituted may be substituted by R⁵ radicals, and preferably have only H in the positions shown as unsubstituted. Among the abovementioned groups, preference is given to the Ar²-1 to Ar²-79 and Ar²-104 to Ar²-220 groups, particular preference to the Ar²-2 to Ar²-47 and Ar²-104 to Ar¹-220 groups. It is very particularly preferable when one or both of the Ar² groups, preferably both of the Ar² groups, are selected from Ar²-2, Ar²-4, Ar²-35, Ar²-37, Ar²-107, Ar²-109, Ar²-110, Ar²-111, Ar²-125, Ar²-126, Ar²-213, Ar²-214, Ar²-215 and Ar²-220. groups.

Preferably, both Ar² are selected from groups of the formulae (Ar²-1) to (Ar²-7) and (Ar²-104) to (Ar²-151), as defined above.

Among the formulae (Ar²-1) to (Ar²-7), preference is given to the formulae (Ar²-1), (Ar²-2), (Ar²-3), (Ar²-4) and (Ar²-5).

In an alternative embodiment, it may preferably be the case that one or both of the Ar² groups, preferably both of the Ar² groups, are selected from the Ar²-2, Ar²-4, Ar²-35, Ar²-37, Ar²-107, Ar²-109, Ar²-110, Ar²-111, Ar²-125, Ar²-126, Ar²-213, Ar²-214, Ar²-215 and Ar²-220 groups, more preferably Ar²-2, Ar²-104 and Ar²-105.

In a preferred embodiment, the Ar² groups do not contain a carbazole group as a substituent R⁵, R⁶ or R⁷.

In a preferred embodiment, V is selected from a bond, O, Si(R⁵)₂ and C(R⁵)₂, preferably a bond, Si(R⁵)₂ and C(R⁵)₂. More preferably, V is a bond. In this case, a carbazole-type structure is formed together with the Ar² groups and the nitrogen atom.

R¹ is preferably the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms; where the alkyl and alkoxy groups mentioned, the aromatic ring systems mentioned and the heteroaromatic ring systems mentioned are each substituted by R⁶ radicals; and where one or more CH₂ groups in the alkyl or alkoxy groups mentioned may be replaced by —C≡C—, —R⁶C═CR⁶—, Si(R⁶)₂, C═O, C═NR⁶, —NR⁶—, —O—, —S—, —C(═O)O— or —C(═O)NR⁶—. More preferably, R¹ is the same or different at each instance and is selected from H, D, F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, where said alkyl groups, said aryl groups and said heteroaryl groups are each substituted by R⁶ radicals.

Preferably, in the compounds of one of the formulae (I) and (II), zero, one, two or three R¹ groups per formula are not H or D. These groups that are not H or D are preferably selected from F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, preferably 5 to 25 aromatic ring atoms, more preferably 6 to 14 aromatic ring atoms, where said alkyl groups, said aryl groups and said heteroaryl groups are each substituted by R⁶ radicals. Preferably none or one of the R¹ groups per formula is not H or D, and more preferably none of the R¹ groups per formula is not H or D.

More preferably, all R¹ radicals in formulae (I) and (II) are H or D, more preferably H.

In an alternative configuration, it may preferably be the case that the compounds of one of the formulae (I) and (II) have at least one R¹ group selected from aromatic ring systems which have 6 to 40 aromatic ring atoms and are substituted by R⁶ radicals; the compounds of one of the formulae (I) and (II) more preferably have at least one R¹ group selected from aryl groups having 6 to 25, preferably 6 to 14, aromatic ring atoms substituted by R⁶ radicals.

In an alternative embodiment, it may more preferably be the case that the compounds of one of the formulae (I) and (II) have at least one R¹ group which is a phenyl group substituted by R⁶ radicals.

R² is preferably the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms; where the alkyl and alkoxy groups mentioned, the aromatic ring systems mentioned and the heteroaromatic ring systems mentioned are each substituted by Re radicals; and where one or more CH₂ groups in the alkyl or alkoxy groups mentioned may be replaced by —C≡C—, —R⁶C═CR⁶—, Si(R⁶)₂, C═O, C═NR⁶, —NR⁶—, —O—, —S—, —C(═O)O— or —C(═O)NR⁶—.

More preferably, R² is the same or different at each instance and is selected from H, D, F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, preferably 5 to 25 aromatic ring atoms, more preferably 6 to 14 aromatic ring atoms, where said alkyl groups, said aryl groups and said heteroaryl groups are each substituted by R⁶ radicals.

Preferably, in the compounds of one of the formulae (I) and (II), zero, one, two or three R² groups per formula are not H or D. These groups that are not H or D are preferably selected from F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, preferably 5 to 25 aromatic ring atoms, more preferably 6 to 14 aromatic atoms, where said alkyl groups, said aryl groups and said heteroaryl groups are each substituted by R⁶ radicals. Preferably none or one of the R² groups per formula is not H or D, and more preferably none of the R² groups per formula is not H or D.

R³ is preferably the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms; where the alkyl and alkoxy groups mentioned, the aromatic ring systems mentioned and the heteroaromatic ring systems mentioned are each substituted by R⁶ radicals; and where one or more CH₂ groups in the alkyl or alkoxy groups mentioned may be replaced by —C≡C—, —R⁶C═CR⁶—, Si(R⁶)₂, C═O, C═NR⁶, —NR⁶—, —O—, —S—, —C(═O)O— or —C(═O)NR⁶—.

Preferably, in the compounds of one of the formulae (I) and (II), zero, one, two or three R³ groups per formula are not H or D. These groups that are not H or D are preferably selected from F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, preferably 5 to 25 aromatic ring atoms, more preferably 6 to 14 aromatic atoms, where said alkyl groups, said aryl groups and said heteroaryl groups are each substituted by R⁶ radicals. Preferably none or one of the R³ groups per formula is not H or D, and more preferably none of the R³ groups per formula is not H or D.

More preferably, all R³ radicals in formulae (I) and (II) are H or D, more preferably H.

In an alternative configuration, it may preferably be the case that the compounds of one of the formulae (I) and (II) have at least one R³ group selected from aromatic ring systems which have 6 to 40 aromatic ring atoms and are substituted by R⁶ radicals; the compounds of one of the formulae (I) and (II) more preferably have at least one R³ group selected from aryl groups having 6 to 25, preferably 6 to 14, aromatic ring atoms substituted by R⁶ radicals.

In an alternative embodiment, it may more preferably be the case that the compounds of one of the formulae (I) and (II) have at least one R³ group which is a phenyl group substituted by R⁶ radicals.

It may preferably be the case that all R¹ and R³ radicals in formulae (I) and (II) are H or D.

R⁴ is preferably the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms; where the alkyl and alkoxy groups mentioned, the aromatic ring systems mentioned and the heteroaromatic ring systems mentioned are each substituted by R⁶ radicals; and where one or more CH₂ groups in the alkyl or alkoxy groups mentioned may be replaced by —C≡C—, —R⁶C═CR⁶—, Si(R⁶)₂, C═O, C═NR⁶, —NR⁶—, —O—, —S—, —C(═O)O— or —C(═O)NR⁶—.

Preferably, in the compounds of one of the formulae (I) and (II), zero, one, two, three or four R⁴ groups per Ar¹ radical are not H or D. These groups that are not H or D are preferably selected from F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, preferably 5 to 25 aromatic ring atoms, more preferably 6 to 14 aromatic atoms, where said alkyl groups, said aryl groups and said heteroaryl groups are each substituted by R⁶ radicals. Preferably none, one or two of the R⁴ groups per formula is not H or D, and more preferably none or one of the R⁴ groups per formula is not H or D.

R⁵ is preferably the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms; where the alkyl and alkoxy groups mentioned, the aromatic ring systems mentioned and the heteroaromatic ring systems mentioned are each substituted by R⁶ radicals; and where one or more CH₂ groups in the alkyl or alkoxy groups mentioned may be replaced by —C≡C—, —R⁶C═CR⁶—, Si(R⁶)₂, C═O, C═NR⁶, —NR⁶—, —O—, —S—, —C(═O)O— or —C(═O)NR⁶—.

Preferably, in the compounds of one of the formulae (I) and (II), zero, one, two, three or four R⁵ groups per Ar² radical are not H or D. These groups that are not H or D are preferably selected from F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, preferably 5 to 25 aromatic ring atoms, more preferably 6 to 14 aromatic atoms, where said alkyl groups, said aryl groups and said heteroaryl groups are each substituted by R⁶ radicals. Preferably none, one or two of the R⁵ groups per formula is not H or D, and more preferably none or one of the R⁵ groups per formula is not H or D.

R⁶ is preferably the same or different at each instance and is selected from H, D, F, CN, Si(R⁷)₃, N(R⁷)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms; where the alkyl and alkoxy groups mentioned, the aromatic ring systems mentioned and the heteroaromatic ring systems mentioned are each substituted by R⁷ radicals; and where one or more CH₂ groups in the alkyl or alkoxy groups mentioned may be replaced by —C≡C—, —R⁷C═CR⁷—, Si(R⁷)₂, C═O, C═NR⁷, —NR⁷—, —O—, —S—, —C(═O)O— or —C(═O)NR⁷—.

Formula (I) preferably conforms to one of the following formulae:

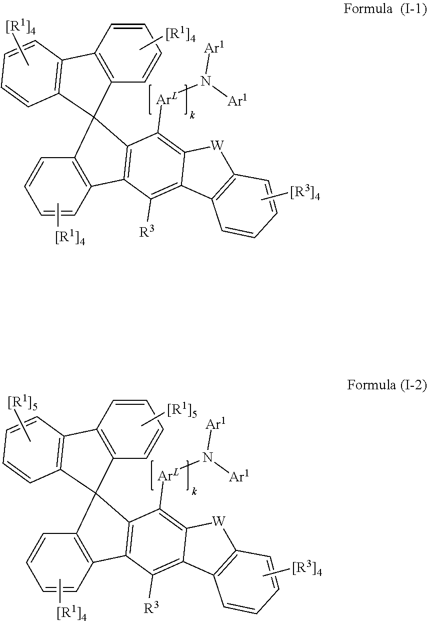

where the groups that occur are as defined above.

Preferred embodiments of the abovementioned formulae conform to the following formulae:

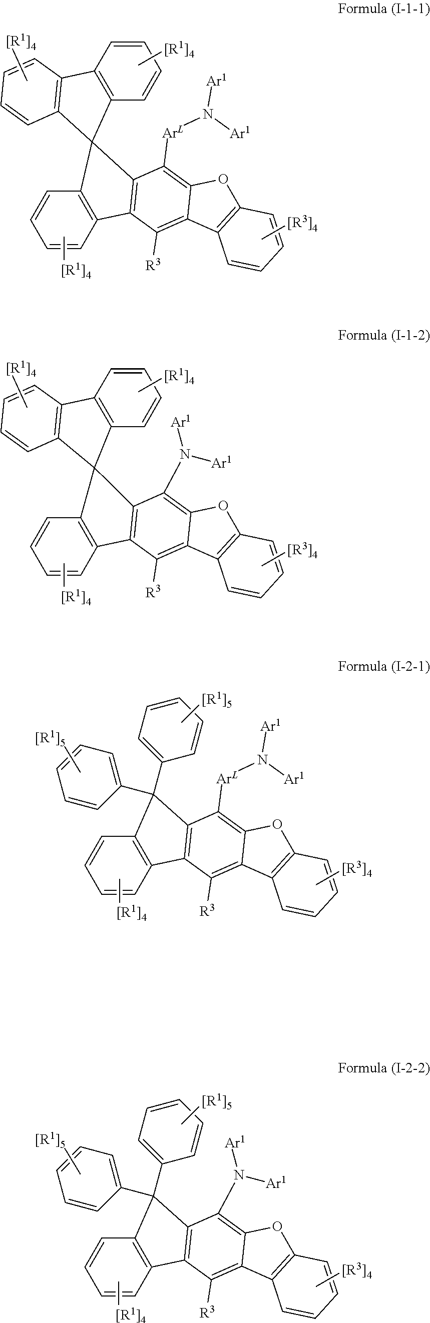

where the groups that occur are as defined above.

Formula (II) preferably conforms to one of the following formulae:

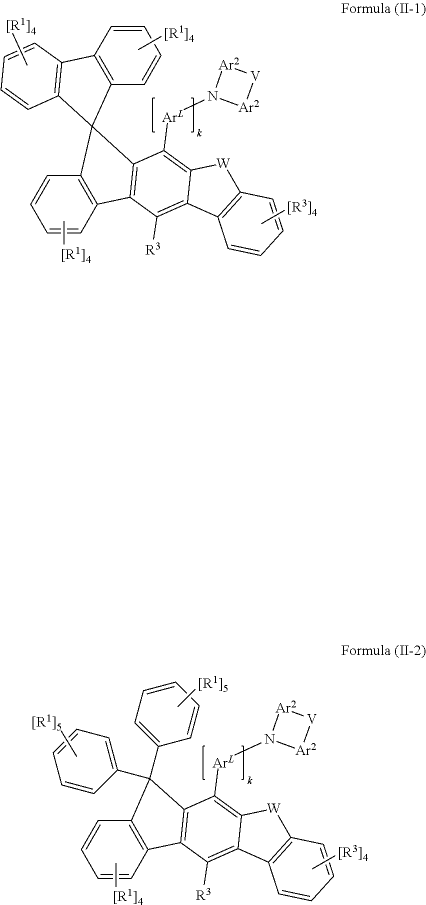

where the groups that occur are as defined above.

Preferred embodiments of the abovementioned formulae conform to the following formulae:

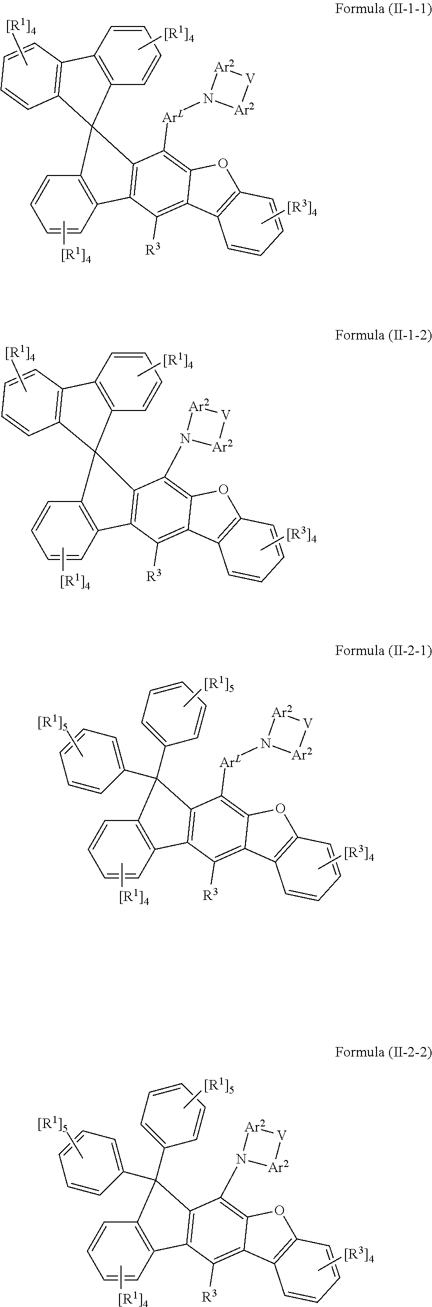

where the groups that occur are as defined above.

Preferred compounds according to the present application are shown below:

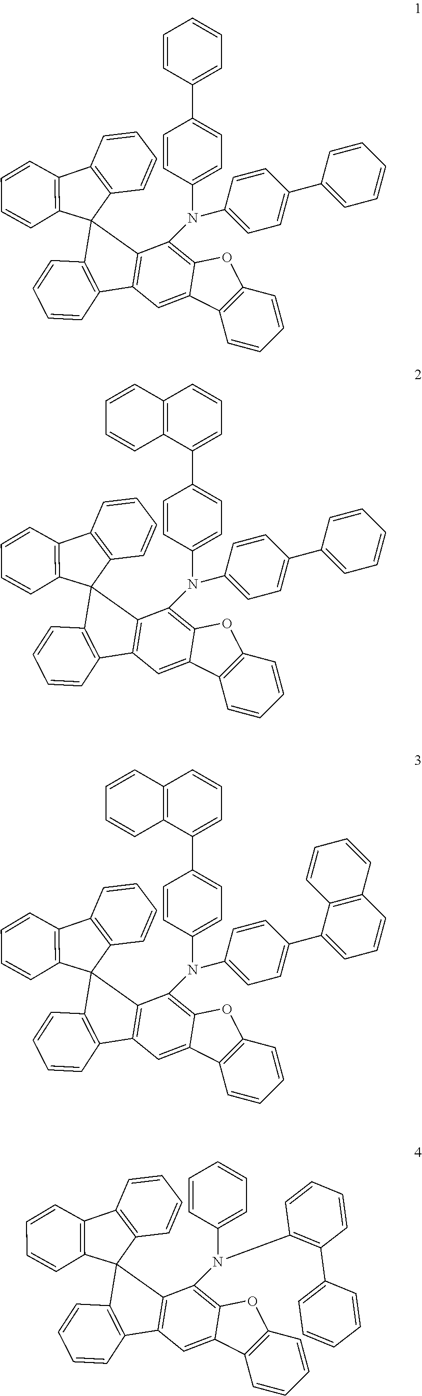 [SMILES: c1ccc(-c2ccc(N(c3ccc(-c4ccccc4)cc3)c3c4c(cc5c3oc3ccccc35)-c3ccccc3C43c4ccccc4-c4ccccc43)cc2)cc1 ; c1ccc(-c2ccc(N(c3ccc(-c4cccc5ccccc45)cc3)c3c4c(cc5c3oc3ccccc35)-c3ccccc3C43c4ccccc4-c4ccccc43)cc2)cc1 ; c1ccc2c(c1)-c1ccccc1C21c2ccccc2-c2cc3c(oc4ccccc43)c(N(c3ccc(-c4cccc5ccccc45)cc3)c3ccc(-c4cccc5ccccc45)cc3)c21 ; c1ccc(-c2ccccc2N(c2ccccc2)c2c3c(cc4c2oc2ccccc24)-c2ccccc2C32c3ccccc3-c3ccccc32)cc1]

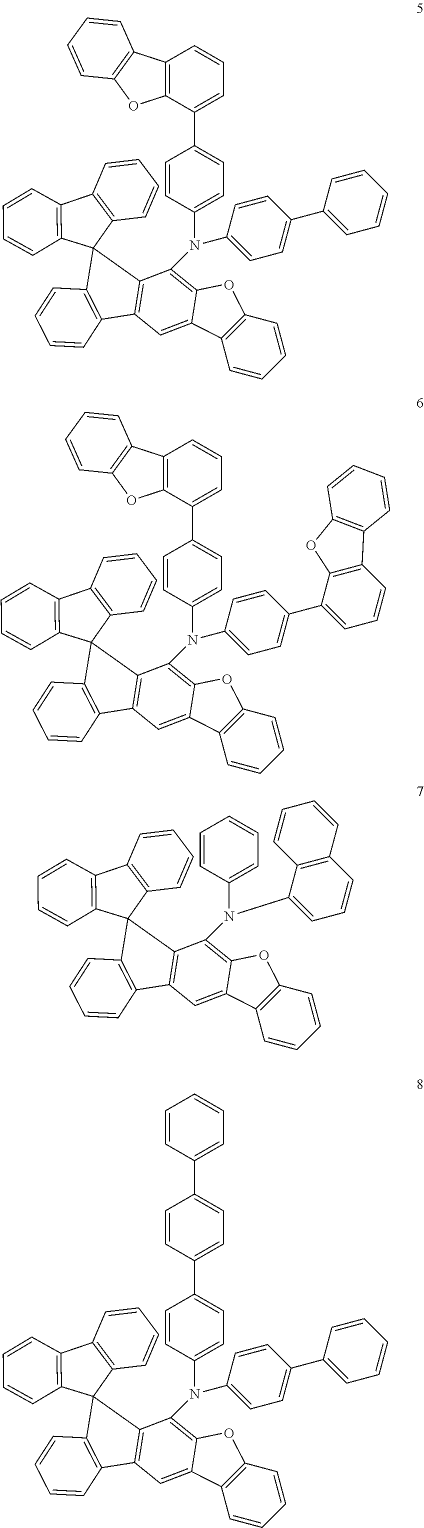

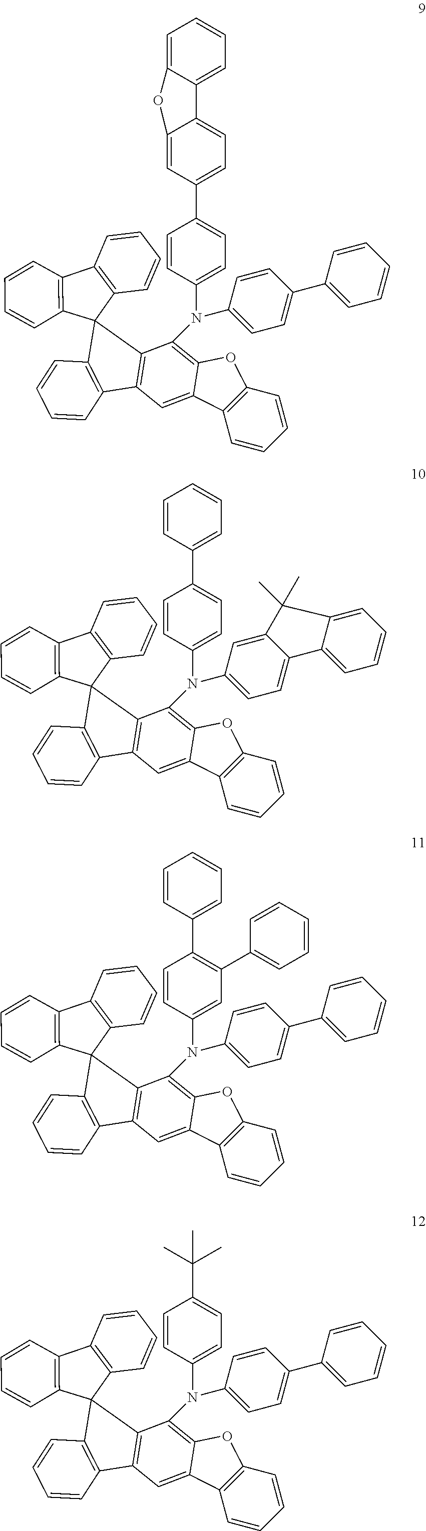 [SMILES: c1ccc(-c2ccc(N(c3ccc(-c4ccc5c(c4)oc4ccccc45)cc3)c3c4c(cc5c3oc3ccccc35)-c3ccccc3C43c4ccccc4-c4ccccc43)cc2)cc1 ; CC1(C)c2ccccc2-c2ccc(N(c3ccc(-c4ccccc4)cc3)c3c4c(cc5c3oc3ccccc35)-c3ccccc3C43c4ccccc4-c4ccccc43)cc21 ; c1ccc(-c2ccc(N(c3ccc(-c4ccccc4)c(-c4ccccc4)c3)c3c4c(cc5c3oc3ccccc35)-c3ccccc3C43c4ccccc4-c4ccccc43)cc2)cc1 ; CC(C)(C)c1ccc(N(c2ccc(-c3ccccc3)cc2)c2c3c(cc4c2oc2ccccc24)-c2ccccc2C32c3ccccc3-c3ccccc32)cc1]

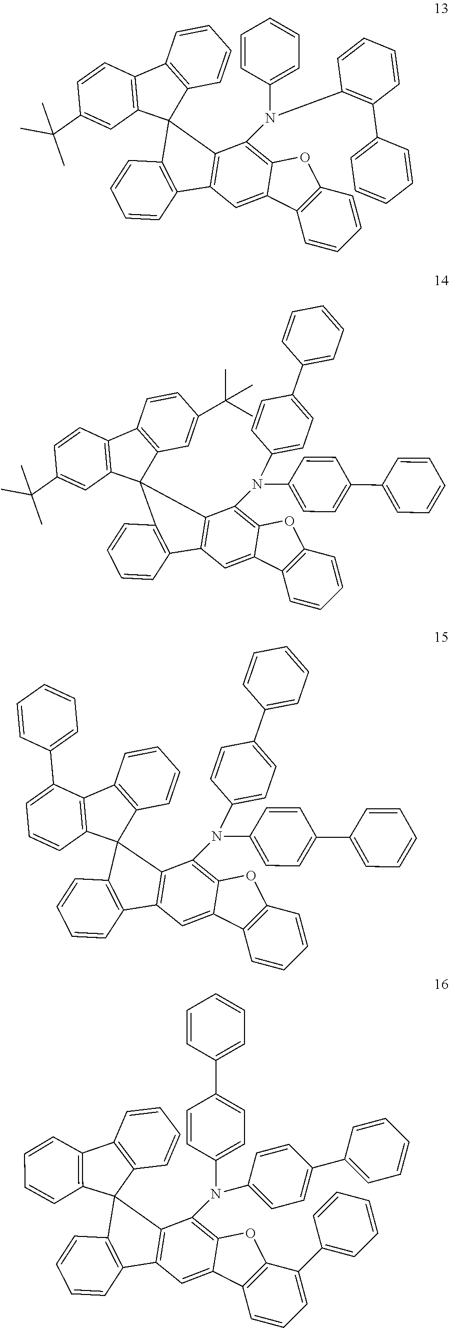

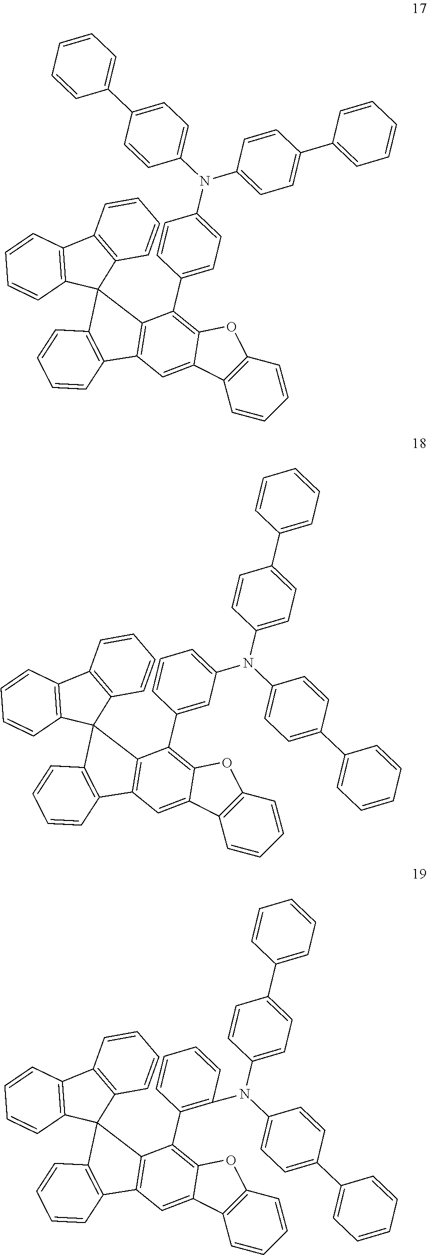 [SMILES: c1ccc(-c2ccc(N(c3ccc(-c4ccccc4)cc3)c3ccc(-c4c5c(cc6c4oc4ccccc46)-c4ccccc4C54c5ccccc5-c5ccccc54)cc3)cc2)cc1 ; c1ccc(-c2ccc(N(c3ccc(-c4ccccc4)cc3)c3cccc(-c4c5c(cc6c4oc4ccccc46)-c4ccccc4C54c5ccccc5-c5ccccc54)c3)cc2)cc1 ; c1ccc(-c2ccc(N(c3ccc(-c4ccccc4)cc3)c3ccccc3-c3c4c(cc5c3oc3ccccc35)-c3ccccc3C43c4ccccc4-c4ccccc43)cc2)cc1]

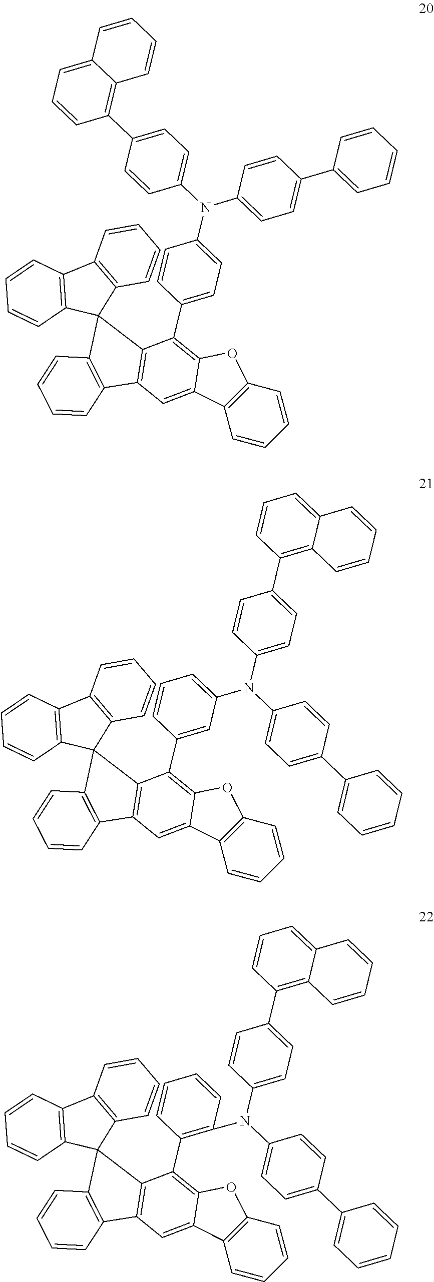 [SMILES: c1ccc(-c2ccc(N(c3ccc(-c4cccc5ccccc45)cc3)c3ccc(-c4c5c(cc6c4oc4ccccc46)-c4ccccc4C54c5ccccc5-c5ccccc54)cc3)cc2)cc1 ; c1ccc(-c2ccc(N(c3ccc(-c4cccc5ccccc45)cc3)c3cccc(-c4c5c(cc6c4oc4ccccc46)-c4ccccc4C54c5ccccc5-c5ccccc54)c3)cc2)cc1 ; c1ccc(-c2ccc(N(c3ccc(-c4cccc5ccccc45)cc3)c3ccccc3-c3c4c(cc5c3oc3ccccc35)-c3ccccc3C43c4ccccc4-c4ccccc43)cc2)cc1]

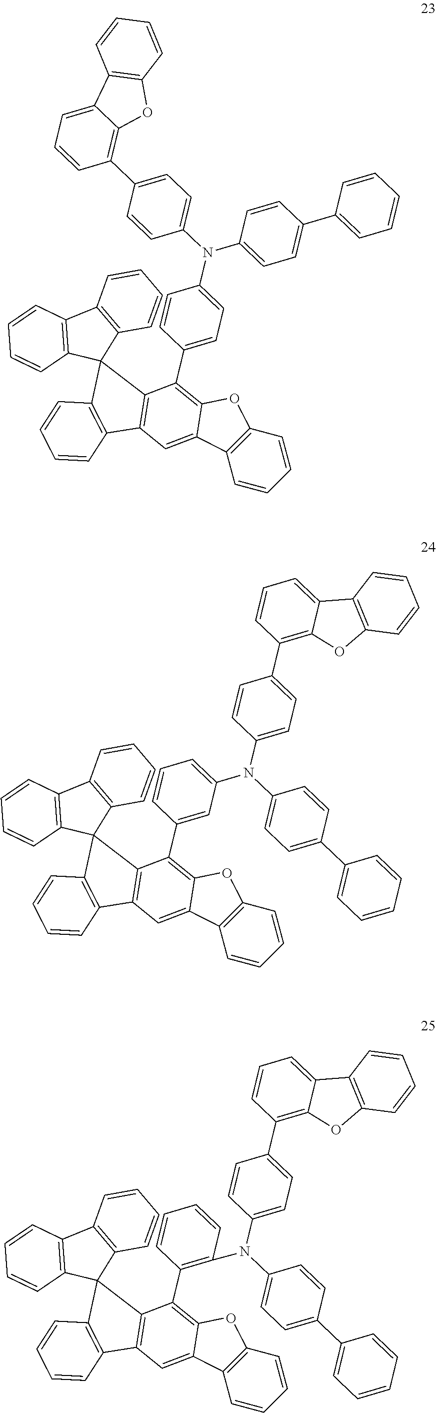 [SMILES: c1ccc(-c2ccc(N(c3ccc(-c4cccc5c4oc4ccccc45)cc3)c3ccc(-c4c5c(cc6c4oc4ccccc46)-c4ccccc4C54c5ccccc5-c5ccccc54)cc3)cc2)cc1 ; c1ccc(-c2ccc(N(c3ccc(-c4cccc5c4oc4ccccc45)cc3)c3cccc(-c4c5c(cc6c4oc4ccccc46)-c4ccccc4C54c5ccccc5-c5ccccc54)c3)cc2)cc1 ; c1ccc(-c2ccc(N(c3ccc(-c4cccc5c4oc4ccccc45)cc3)c3ccccc3-c3c4c(cc5c3oc3ccccc35)-c3ccccc3C43c4ccccc4-c4ccccc43)cc2)cc1]

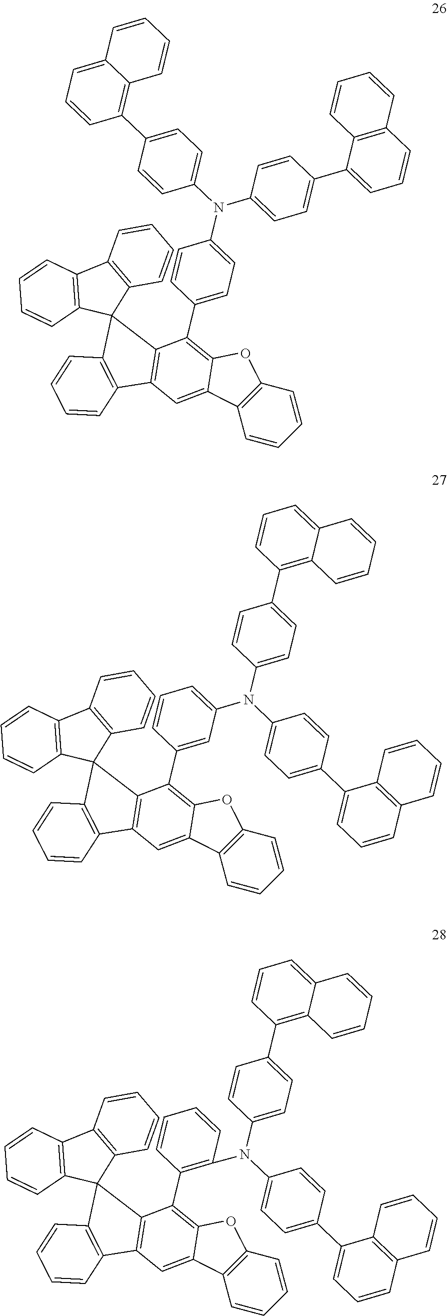 [SMILES: c1ccc2c(c1)-c1ccccc1C21c2ccccc2-c2cc3c(oc4ccccc43)c(-c3ccc(N(c4ccc(-c5cccc6ccccc56)cc4)c4ccc(-c5cccc6ccccc56)cc4)cc3)c21 ; c1cc(-c2c3c(cc4c2oc2ccccc24)-c2ccccc2C32c3ccccc3-c3ccccc32)cc(N(c2ccc(-c3cccc4ccccc34)cc2)c2ccc(-c3cccc4ccccc34)cc2)c1 ; c1ccc(N(c2ccc(-c3cccc4ccccc34)cc2)c2ccc(-c3cccc4ccccc34)cc2)c(-c2c3c(cc4c2oc2ccccc24)-c2ccccc2C32c3ccccc3-c3ccccc32)c1]

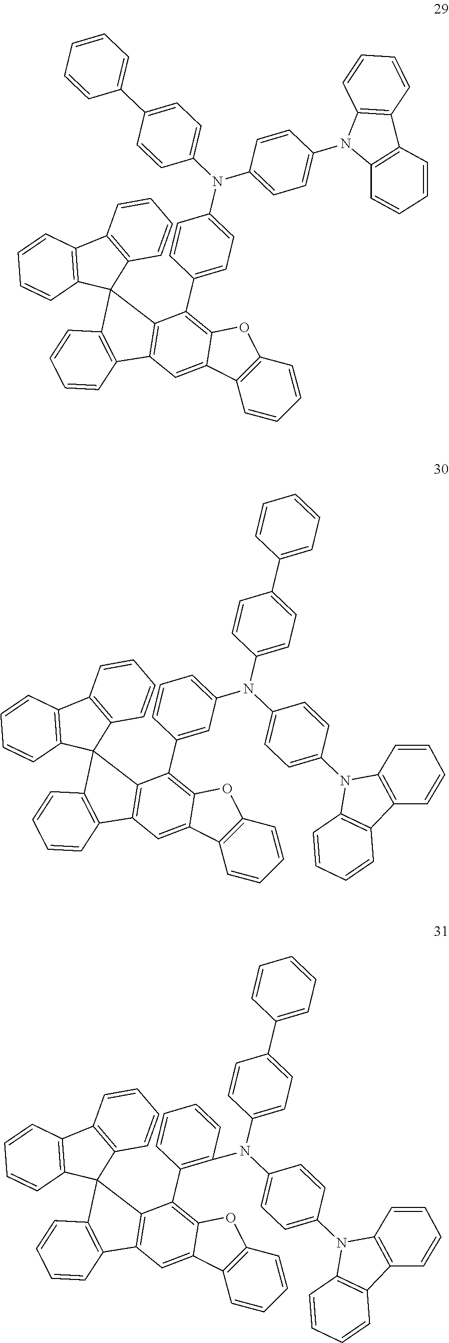 [SMILES: c1ccc(-c2ccc(N(c3ccc(-c4c5c(cc6c4oc4ccccc46)-c4ccccc4C54c5ccccc5-c5ccccc54)cc3)c3ccc(-n4c5ccccc5c5ccccc54)cc3)cc2)cc1 ; c1ccc(-c2ccc(N(c3ccc(-n4c5ccccc5c5ccccc54)cc3)c3cccc(-c4c5c(cc6c4oc4ccccc46)-c4ccccc4C54c5ccccc5-c5ccccc54)c3)cc2)cc1 ; c1ccc(-c2ccc(N(c3ccc(-n4c5ccccc5c5ccccc54)cc3)c3ccccc3-c3c4c(cc5c3oc3ccccc35)-c3ccccc3C43c4ccccc4-c4ccccc43)cc2)cc1]

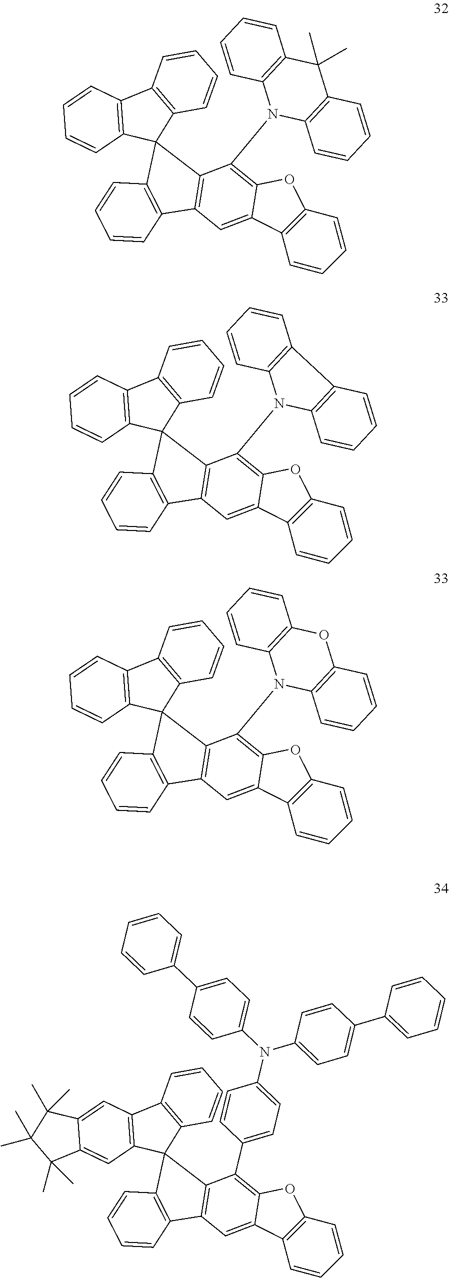

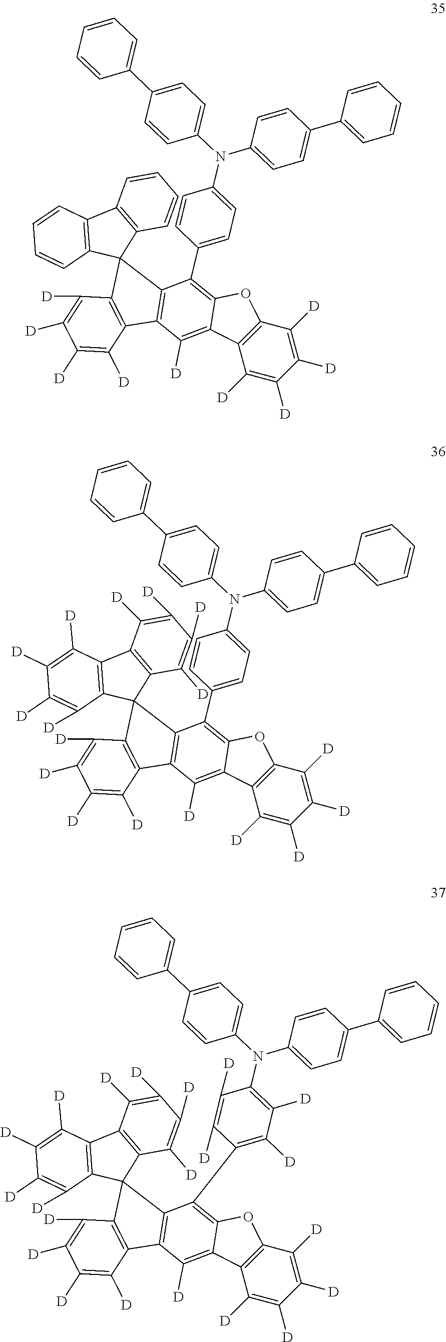

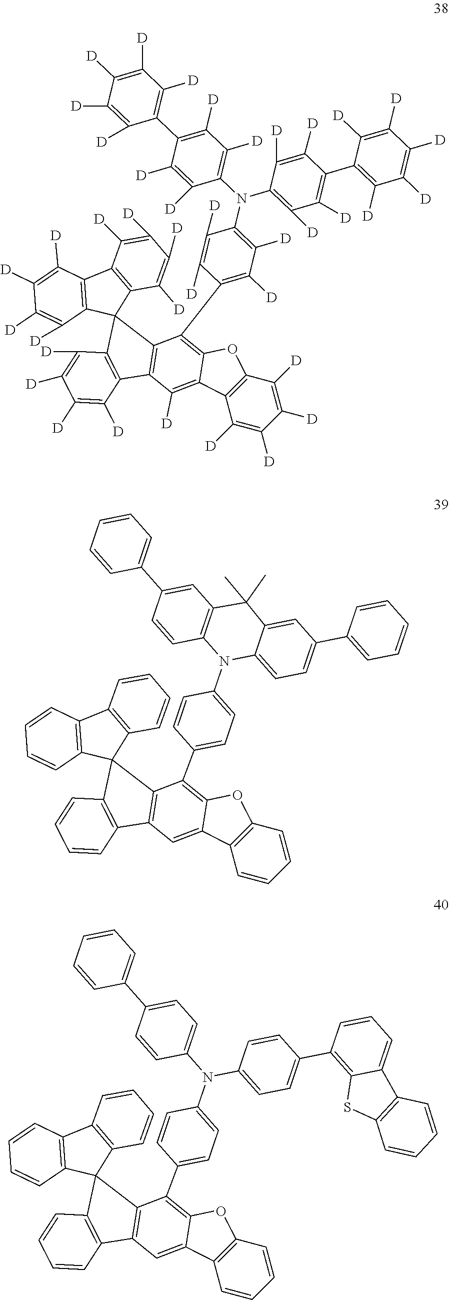

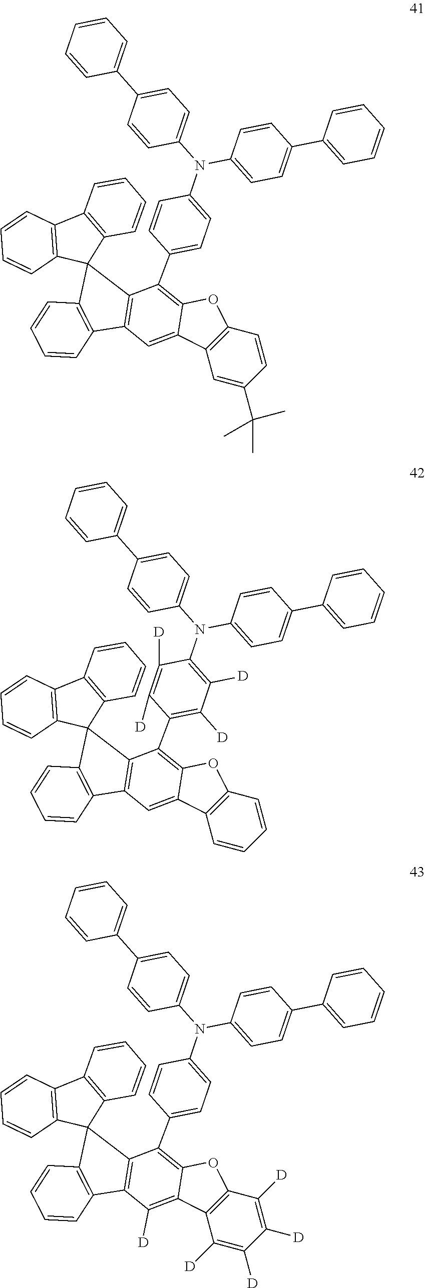 [SMILES: CC(C)(C)c1ccc2oc3c(-c4ccc(N(c5ccc(-c6ccccc6)cc5)c5ccc(-c6ccccc6)cc5)cc4)c4c(cc3c2c1)-c1ccccc1C41c2ccccc2-c2ccccc21 ; [2H]c1c([2H])c(N(c2ccc(-c3ccccc3)cc2)c2ccc(-c3ccccc3)cc2)c([2H])c([2H])c1-c1c2c(cc3c1oc1ccccc13)-c1ccccc1C21c2ccccc2-c2ccccc21 ; [2H]c1c([2H])c([2H])c2c(oc3c(-c4ccc(N(c5ccc(-c6ccccc6)cc5)c5ccc(-c6ccccc6)cc5)cc4)c4c(c([2H])c32)-c2ccccc2C42c3ccccc3-c3ccccc32)c1[2H]]

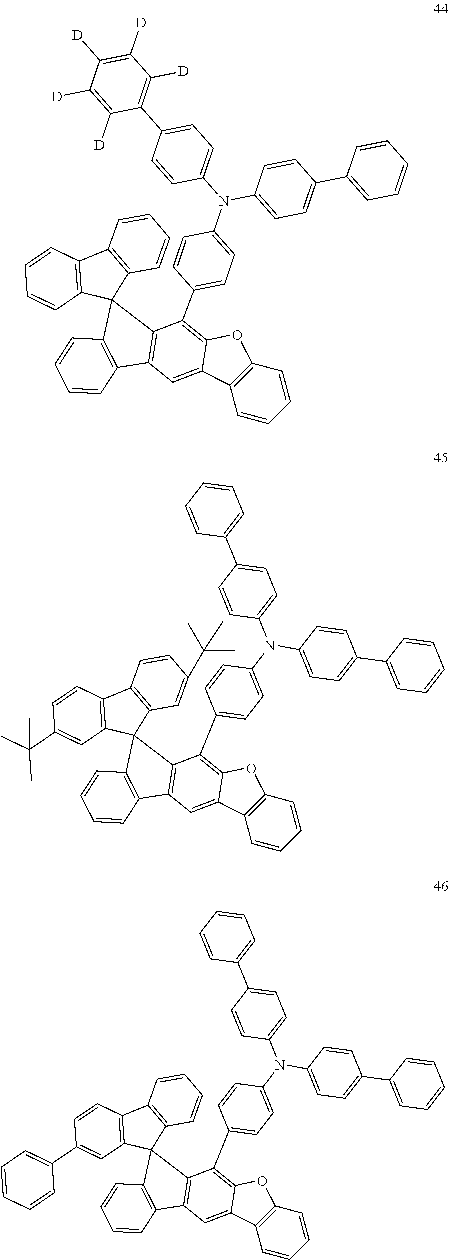 [SMILES: [2H]c1c([2H])c([2H])c(-c2ccc(N(c3ccc(-c4ccccc4)cc3)c3ccc(-c4c5c(cc6c4oc4ccccc46)-c4ccccc4C54c5ccccc5-c5ccccc54)cc3)cc2)c([2H])c1[2H] ; CC(C)(C)c1ccc2c(c1)C1(c3cc(C(C)(C)C)ccc3-2)c2ccccc2-c2cc3c(oc4ccccc43)c(-c3ccc(N(c4ccc(-c5ccccc5)cc4)c4ccc(-c5ccccc5)cc4)cc3)c21 ; c1ccc(-c2ccc(N(c3ccc(-c4ccccc4)cc3)c3ccc(-c4c5c(cc6c4oc4ccccc46)-c4ccccc4C54c5ccccc5-c5ccc(-c6ccccc6)cc54)cc3)cc2)cc1]

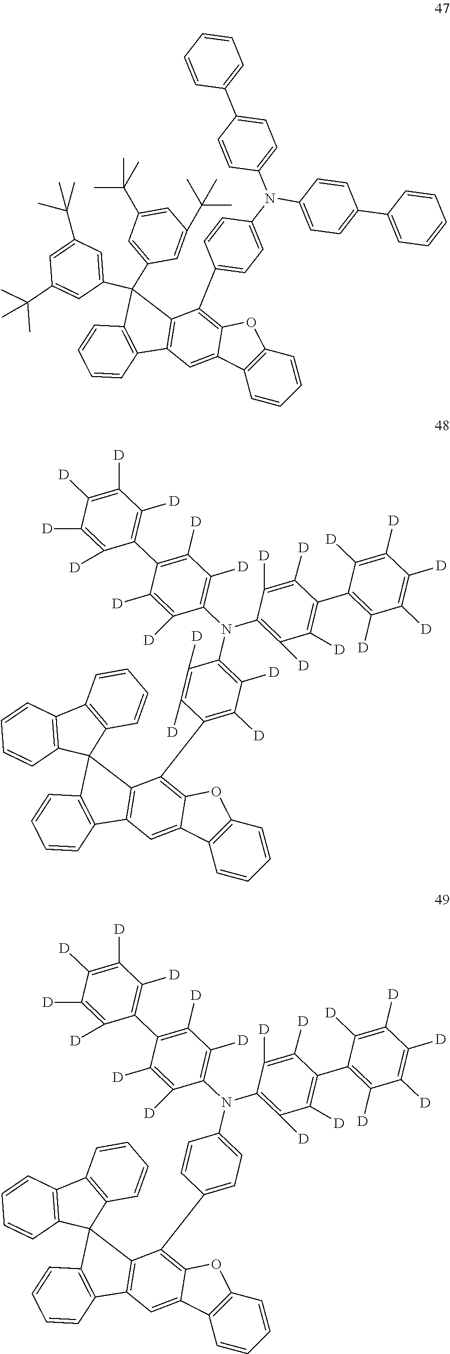

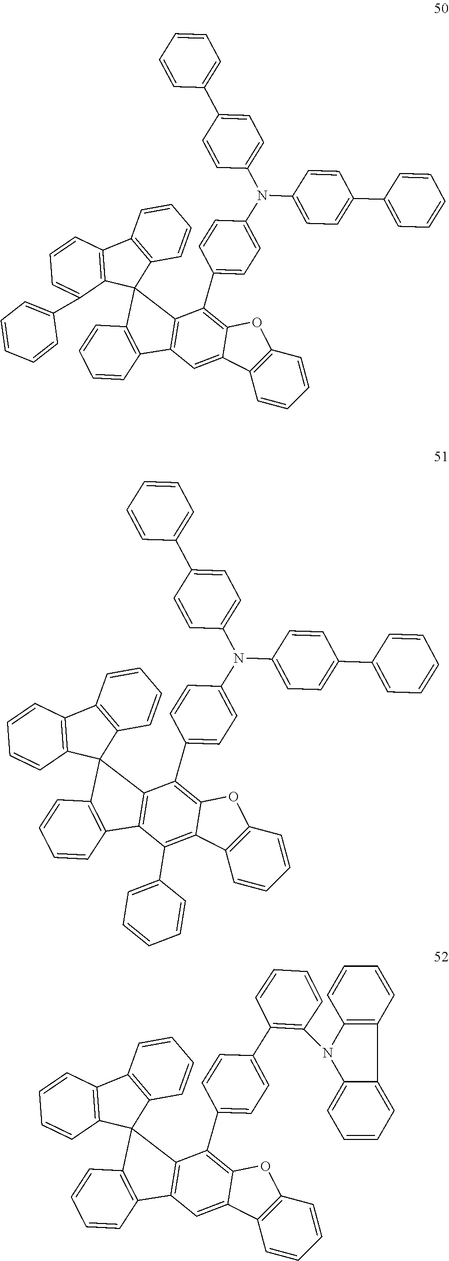 [SMILES: c1ccc(-c2ccc(N(c3ccc(-c4ccccc4)cc3)c3ccc(-c4c5c(cc6c4oc4ccccc46)-c4ccccc4C54c5ccccc5-c5cccc(-c6ccccc6)c54)cc3)cc2)cc1 ; c1ccc(-c2ccc(N(c3ccc(-c4ccccc4)cc3)c3ccc(-c4c5c(c(-c6ccccc6)c6c4oc4ccccc46)-c4ccccc4C54c5ccccc5-c5ccccc54)cc3)cc2)cc1 ; c1ccc(-n2c3ccccc3c3ccccc32)c(-c2ccc(-c3c4c(cc5c3oc3ccccc35)-c3ccccc3C43c4ccccc4-c4ccccc43)cc2)c1]

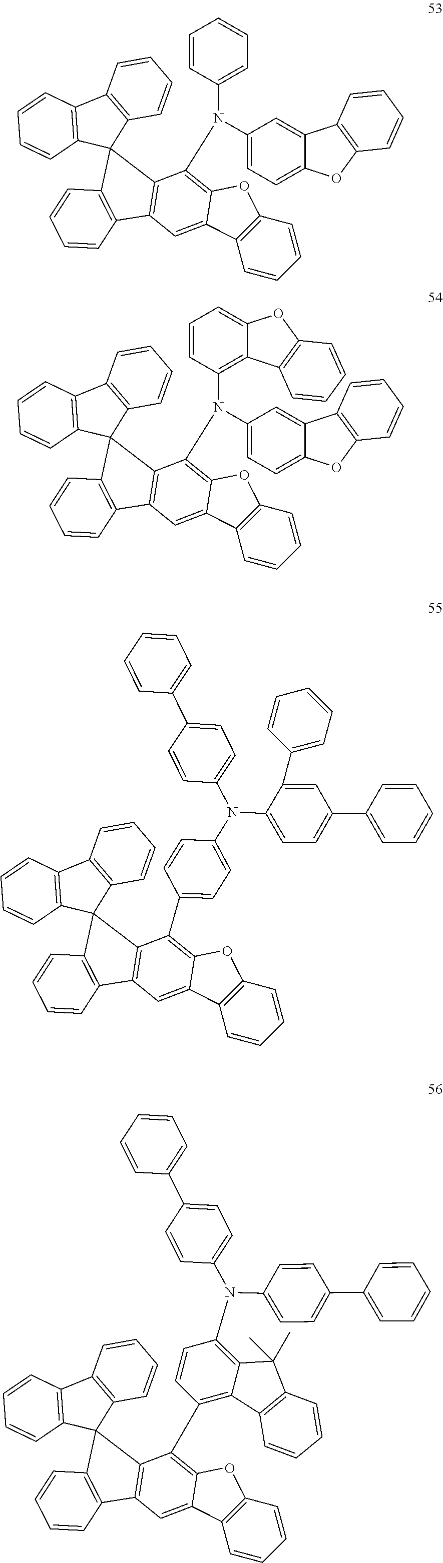

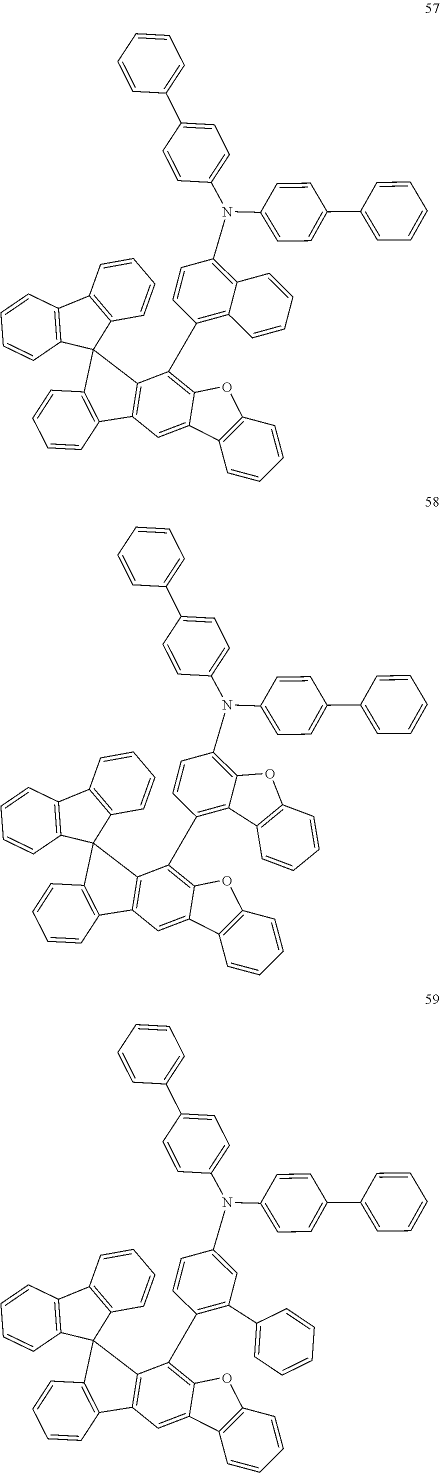 [SMILES: c1ccc(-c2ccc(N(c3ccc(-c4ccccc4)cc3)c3ccc(-c4c5c(cc6c4oc4ccccc46)-c4ccccc4C54c5ccccc5-c5ccccc54)c4ccccc34)cc2)cc1 ; c1ccc(-c2ccc(N(c3ccc(-c4ccccc4)cc3)c3ccc(-c4c5c(cc6c4oc4ccccc46)-c4ccccc4C54c5ccccc5-c5ccccc54)c4c3oc3ccccc34)cc2)cc1 ; c1ccc(-c2ccc(N(c3ccc(-c4ccccc4)cc3)c3ccc(-c4c5c(cc6c4oc4ccccc46)-c4ccccc4C54c5ccccc5-c5ccccc54)c(-c4ccccc4)c3)cc2)cc1]

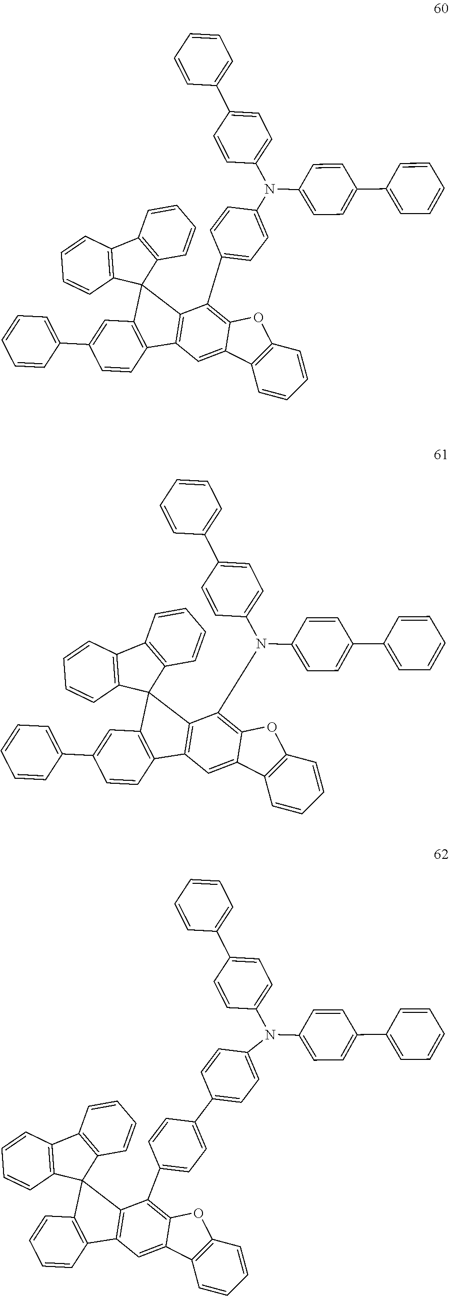 [SMILES: c1ccc(-c2ccc(N(c3ccc(-c4ccccc4)cc3)c3ccc(-c4c5c(cc6c4oc4ccccc46)-c4ccc(-c6ccccc6)cc4C54c5ccccc5-c5ccccc54)cc3)cc2)cc1 ; c1ccc(-c2ccc(N(c3ccc(-c4ccccc4)cc3)c3c4c(cc5c3oc3ccccc35)-c3ccc(-c5ccccc5)cc3C43c4ccccc4-c4ccccc43)cc2)cc1 ; c1ccc(-c2ccc(N(c3ccc(-c4ccccc4)cc3)c3ccc(-c4ccc(-c5c6c(cc7c5oc5ccccc57)-c5ccccc5C65c6ccccc6-c6ccccc65)cc4)cc3)cc2)cc1]

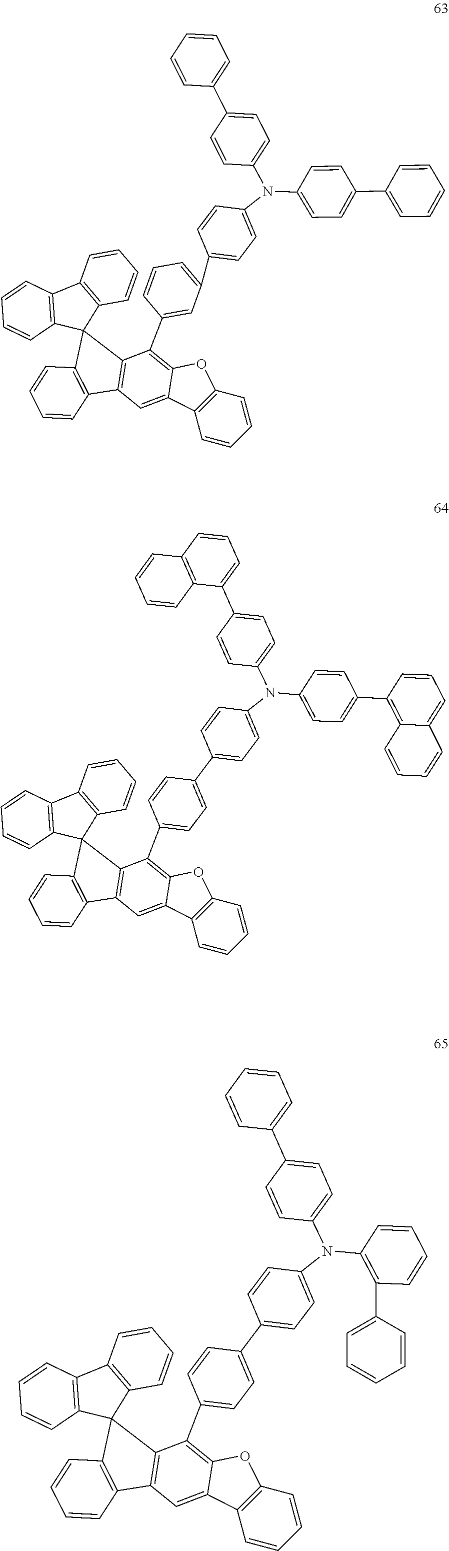 [SMILES: c1ccc(-c2ccc(N(c3ccc(-c4ccccc4)cc3)c3ccc(-c4cccc(-c5c6c(cc7c5oc5ccccc57)-c5ccccc5C65c6ccccc6-c6ccccc65)c4)cc3)cc2)cc1 ; c1ccc2c(c1)-c1ccccc1C21c2ccccc2-c2cc3c(oc4ccccc43)c(-c3ccc(-c4ccc(N(c5ccc(-c6cccc7ccccc67)cc5)c5ccc(-c6cccc7ccccc67)cc5)cc4)cc3)c21 ; c1ccc(-c2ccc(N(c3ccc(-c4ccc(-c5c6c(cc7c5oc5ccccc57)-c5ccccc5C65c6ccccc6-c6ccccc65)cc4)cc3)c3ccccc3-c3ccccc3)cc2)cc1]

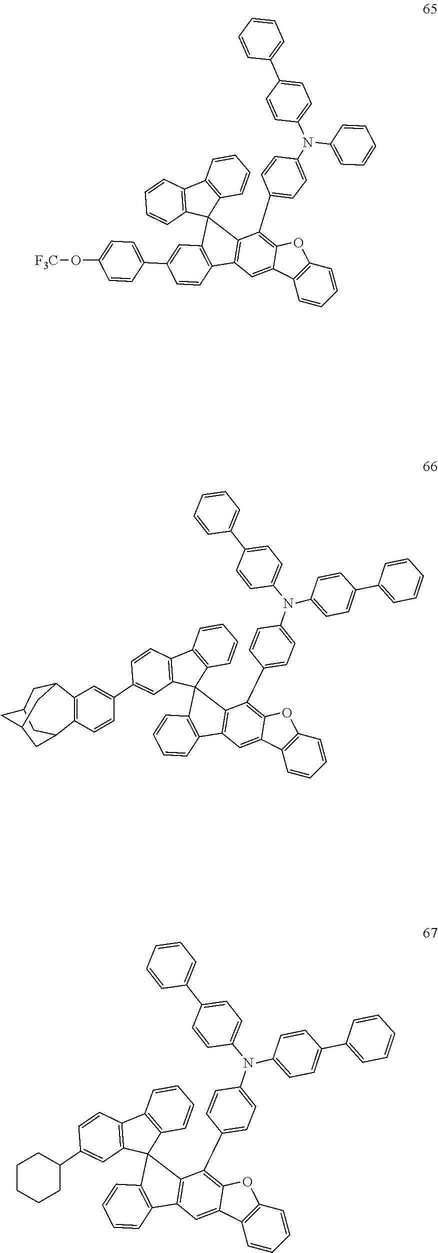 [SMILES: FC(F)(F)Oc1ccc(-c2ccc3c(c2)C2(c4ccccc4-c4ccccc42)c2c-3cc3c(oc4ccccc43)c2-c2ccc(N(c3ccccc3)c3ccc(-c4ccccc4)cc3)cc2)cc1 ; c1ccc(-c2ccc(N(c3ccc(-c4ccccc4)cc3)c3ccc(-c4c5c(cc6c4oc4ccccc46)-c4ccccc4C54c5ccccc5-c5ccc(-c6ccc7c(c6)C6CC8CC(CC7C8)C6)cc54)cc3)cc2)cc1 ; c1ccc(-c2ccc(N(c3ccc(-c4ccccc4)cc3)c3ccc(-c4c5c(cc6c4oc4ccccc46)-c4ccccc4C54c5ccccc5-c5ccc(C6CCCCC6)cc54)cc3)cc2)cc1]

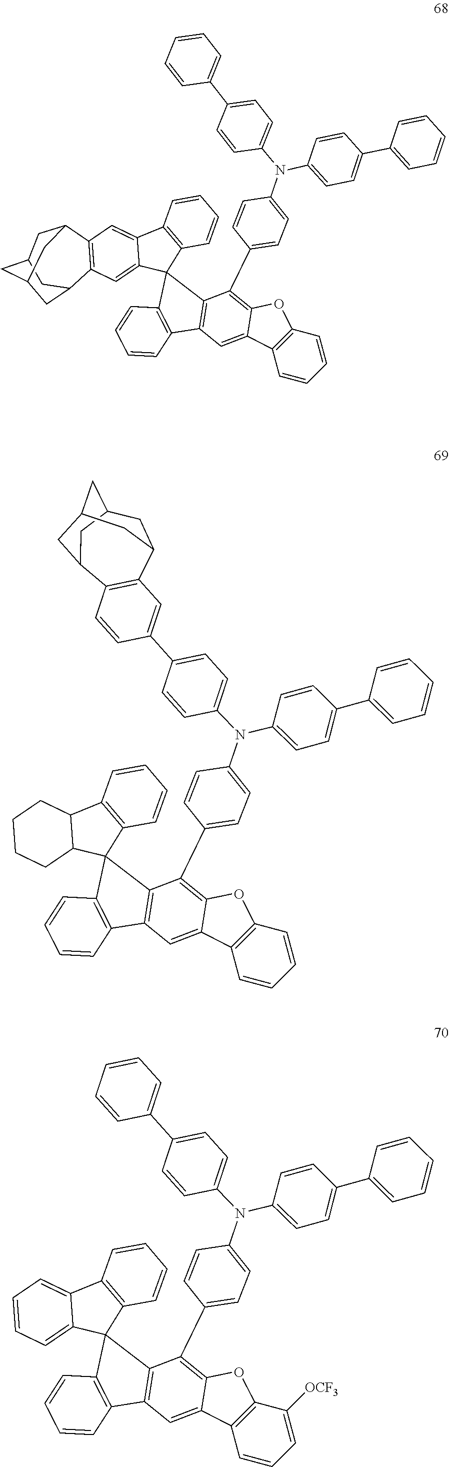 [SMILES: c1ccc(-c2ccc(N(c3ccc(-c4ccccc4)cc3)c3ccc(-c4c5c(cc6c4oc4ccccc46)-c4ccccc4C54c5ccccc5-c5cc6c(cc54)C4CC5CC(CC6C5)C4)cc3)cc2)cc1 ; c1ccc(-c2ccc(N(c3ccc(-c4ccc5c(c4)C4CC6CC(CC5C6)C4)cc3)c3ccc(-c4c5c(cc6c4oc4ccccc46)-c4ccccc4C54c5ccccc5C5CCCCC54)cc3)cc2)cc1 ; FC(F)(F)Oc1cccc2c1oc1c(-c3ccc(N(c4ccc(-c5ccccc5)cc4)c4ccc(-c5ccccc5)cc4)cc3)c3c(cc12)-c1ccccc1C31c2ccccc2-c2ccccc21]

The compounds according to the present application may be prepared by means of the synthesis methods described hereinafter.

According to the method shown in diagram 1, a Hartwig-Buchwald coupling can proceed from an indenodibenzofuran derivative, by means of which an amino group is introduced into the molecule. This affords a compound according to the present application in which the index k=0.

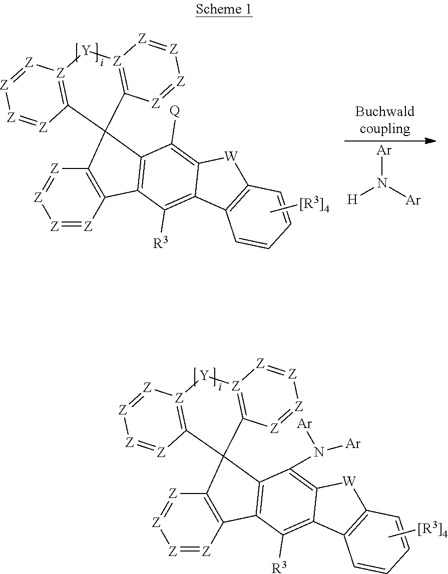

An alternative, as shown in schemes 2 and 3, is a Suzuki coupling, by means of which an aromatic ring system is introduced into the molecule. This affords a compound according to the present application in which the index k>0.

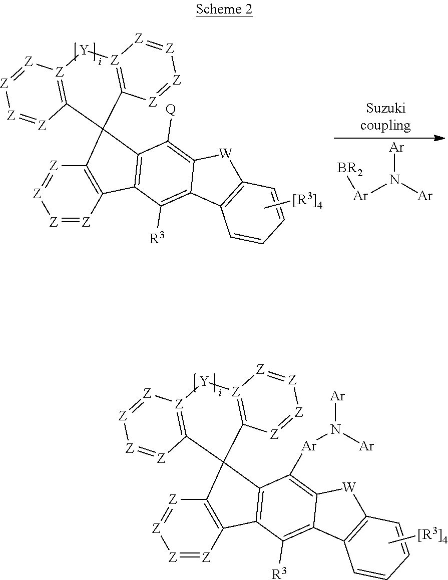

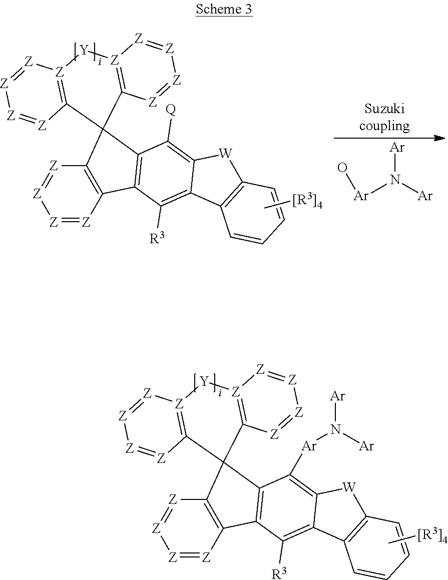

The definitions of the variable groups in the schemes shown above are as defined above, where the further groups are as follows:
R=H or organic radical
Q=reactive group
Ar=optionally substituted aromatic or heteroaromatic, corresponding to the above-defined Ar^{L}, Ar¹, Ar² and V groups.

The present application thus provides a process for preparing a compound according to the present application, characterized in that an indenodibenzofuran derivative a) substituted by a reactive group is reacted in a coupling reaction with a secondary amine, or b) is reacted in a coupling reaction with an aromatic or heteroaromatic species bearing a reactive group. In variant b), the reactive group on the indenodibenzofuran derivative preferably contains boron, and the reactive group on the aromatic or heteroaromatic system is preferably selected from Cl, Br and I. Alternatively, the reactive group on the indenodibenzofuran derivative is selected from Cl, Br and I, and the reactive group on the aromatic or heteroaromatic system preferably contains boron. In a first embodiment of variant b), an indenodibenzofuran derivative substituted by a reactive group is reacted in a coupling reaction with an aromatic or heteroaromatic system bearing a boron-containing group. In a second embodiment of variant b), an indenodibenzofuran derivative substituted by a boron-containing group is reacted in a coupling reaction with an aromatic or heteroaromatic system bearing a reactive group.

Reactive groups are known to the skilled person for the specific reaction. One of the reactive groups is preferably selected from Cl, Br and I, more preferably from Br and I. The coupling reaction in the reaction under a) is preferably a Hartwig-Buchwald coupling reaction. The coupling reaction under b) is preferably a Suzuki coupling reaction. In a Suzuki coupling reaction, a reactive group is preferably selected as set out above and a further reactive group is preferably a boron atom-containing group, preferably a boric acid or boric ester group.

The indenodibenzofuran derivative substituted by a reactive group is preferably prepared from a dibenzofuran derivative substituted by two reactive groups, which is reacted with a carbonyl compound in an organometallic addition reaction.

Compounds having a boron atom-containing group, preferably a boric acid or boric ester group, can preferably be obtained by reaction with organometallic compounds, preferably organometallic lithium compounds. It is also possible here to use unsubstituted indenodibenzofuran derivatives. In addition, unsubstituted indenodibenzofuran derivatives can also be used to obtain indenodibenzofuran derivatives with a reactive group selected from Cl, Br and I, more preferably from Br and I, by reaction with organometallic compounds, preferably organometallic lithium compounds.

The above-detailed process procedures and synthesis stages are disclosed inter alia in documents WO 2015/090504 and WO 2015/022051 A1. These publications are hereby explicitly incorporated.

The above-described compounds of the invention, especially compounds substituted by reactive leaving groups, such as bromine, iodine, chlorine, boronic acid or boronic ester, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C—C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of formula (I) or (II), wherein the bond(s) to the polymer, oligomer or dendrimer may be localized at any desired positions substituted by R¹, R², R³, R⁴ or R⁵ in formula (I) or (II). According to the linkage of the compound of formula (I) or (II), the compound is part of a side chain of the oligomer or polymer or part of the main chain. An oligomer in the context of this invention is understood to mean a compound formed from at least three monomer units. A polymer in the context of the invention is understood to mean a compound formed from at least ten monomer units. The polymers, oligomers or dendrimers of the invention may be conjugated, partly conjugated or nonconjugated. The oligomers or polymers of the invention may be linear, branched or dendritic. In the structures having linear linkage, the units of formula (I) or (II) may be joined directly to one another, or they may be joined to one another via a bivalent group, for example via a substituted or unsubstituted alkylene group, via a heteroatom or via a bivalent aromatic or heteroaromatic group.

In branched and dendritic structures, it is possible, for example, for three or more units of formula (I) or (II) to be joined via a trivalent or higher-valency group, for example via a trivalent or higher-valency aromatic or heteroaromatic group, to give a branched or dendritic oligomer or polymer.

For the repeat units of formula (I) or (II) in oligomers, dendrimers and polymers, the same preferences apply as described above for compounds of formula (I) or (II).

For preparation of the oligomers or polymers, the monomers of the invention are homopolymerized or copolymerized with further monomers. Suitable and preferred comonomers are selected from fluorenes, spirobifluorenes, paraphenylenes, carbazoles, thiophenes, dihydrophenanthrenes, cis- and trans-indenofluorenes, ketones, phenanthrenes or else two or more of these units. The polymers, oligomers and dendrimers typically contain still further units, for example emitting (fluorescent or phosphorescent) units, for example vinyltriarylamines or phosphorescent metal complexes, and/or charge transport units, especially those based on triarylamines.

The polymers, oligomers and dendrimers of the invention have advantageous properties, especially high lifetimes, high efficiencies and good colour coordinates.

The polymers and oligomers of the invention are generally prepared by polymerization of one or more monomer types, of which at least one monomer leads to repeat units of the formula (I) or (II) in the polymer. Suitable polymerization reactions are known to those skilled in the art and are described in the literature. Particularly suitable and preferred polymerization reactions which lead to C—C and C—N couplings are as follows:
(A) SUZUKI polymerization;
(B) YAMAMOTO polymerization;
(C) STILLE polymerization; and
(D) HARTWIG-BUCHWALD polymerization.

How the polymerization can be conducted by these methods and how the polymers can then be separated from the reaction medium and purified is known to those skilled in the art and is described in detail in the literature.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, alpha-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, or mixtures of these solvents.

The invention therefore further provides a formulation, especially a solution, dispersion or emulsion, comprising at least one compound of formula (I) or (II) or at least one polymer, oligomer or dendrimer containing at least one unit of formula (I) or (II) and at least one solvent, preferably an organic solvent. The way in which such solutions can be prepared is known to those skilled in the art.

The compound of formula (I) or (II) is suitable for use in an electronic device, especially an organic electroluminescent device (OLED). Depending on the substitution, the compound of the formula (I) or (II) can be used in different functions and layers. Preference is given to use as a hole-transporting material in a hole-transporting layer and/or as matrix material in an emitting layer, more preferably in combination with a phosphorescent emitter.

The invention therefore further provides for the use of a compound of formula (I) or (II) in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides an electronic device comprising at least one compound of formula (I) or (II). This electronic device is preferably selected from the abovementioned devices.

Particular preference is given to an organic electroluminescent device comprising anode, cathode and at least one emitting layer, characterized in that at least one organic layer comprising at least one compound of formula (I) or (II) is present in the device. Preference is given to an organic electroluminescent device comprising anode, cathode and at least one emitting layer, characterized in that at least one organic layer in the device, selected from hole-transporting and emitting layers, comprises at least one compound of formula (I) or (II).

A hole-transporting layer is understood here to mean all layers disposed between anode and emitting layer, preferably hole injection layer, hole transport layer and electron blocker layer. A hole injection layer is understood here to mean a layer that directly adjoins the anode. A hole transport layer is understood here to mean a layer which is between the anode and emitting layer but does not directly adjoin the anode, and preferably does not directly adjoin the emitting layer either. An electron blocker layer is understood here to mean a layer which is between the anode and emitting layer and directly adjoins the emitting layer. An electron blocker layer preferably has a high-energy LUMO and hence prevents electrons from exiting from the emitting layer.

Apart from the cathode, anode and emitting layer, the electronic device may comprise further layers. These are selected, for example, from in each case one or more hole injection layers, hole transport layers, hole blocker layers, electron transport layers, electron injection layers, electron blocker layers, exciton blocker layers, interlayers, charge generation layers and/or organic or inorganic p/n junctions. However, it should be pointed out that not every one of these layers need necessarily be present and the choice of layers always depends on the compounds used and especially also on whether the device is a fluorescent or phosphorescent electroluminescent device.

The sequence of layers in the electronic device is preferably as follows:
-anode-
-hole injection layer-
-hole transport layer-
-optionally further hole transport layers-
-emitting layer-
-optionally hole blocker layer-
-electron transport layer-
-electron injection layer-
-cathode-.

At the same time, it should be pointed out again that not all the layers mentioned need be present and/or that further layers may additionally be present.

The organic electroluminescent device of the invention may contain two or more emitting layers. More preferably, these emission layers have several emission maxima between 380 nm and 750 nm overall, such that the overall result is white emission; in other words, various emitting compounds which may fluoresce or phosphoresce and which emit blue, green, yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. The compounds of the invention here are preferably present in a hole-transporting layer or in the emitting layer. It should be noted that, for the production of white light, rather than a plurality of color-emitting emitter compounds, an emitter compound used individually which emits over a broad wavelength range may also be suitable.

It is preferable that the compound of the formula (I) or (II) is used as hole transport material. The emitting layer here may be a fluorescent emitting layer, or it may be a phosphorescent emitting layer. The emitting layer is preferably a blue-fluorescing layer or a green-phosphorescing layer.

When the device containing the compound of the formula (I) or (II) contains a phosphorescent emitting layer, it is preferable that this layer contains two or more, preferably exactly two, different matrix materials (mixed matrix system). Preferred embodiments of mixed matrix systems are described in detail further down.

If the compound of formula (I) or (II) is used as hole transport material in a hole transport layer, a hole injection layer or an electron blocker layer, the compound can be used as pure material, i.e. in a proportion of 100%, in the hole transport layer, or it can be used in combination with one or more further compounds.

In a preferred embodiment, a hole-transporting layer comprising the compound of the formula (I) or (II) additionally comprises one or more further hole-transporting compounds. These further hole-transporting compounds are preferably selected from triarylamine compounds, more preferably from monotriarylamine compounds. They are most preferably selected from the preferred embodiments of hole transport materials that are specified further down. In the preferred embodiment described, the compound of the formula (I) or (II) and the one or more further hole-transporting compounds are preferably each present in a proportion of at least 10%, more preferably each in a proportion of at least 20%.

In a preferred embodiment, a hole-transporting layer comprising the compound of the formula (I) or (II) additionally contains one or more p-dopants. p-Dopants used according to the present invention are preferably those organic electron acceptor compounds capable of oxidizing one or more of the other compounds in the mixture.

Particularly preferred as p-dopants are quinodimethane compounds, azaindenofluorenediones, azaphenalenes, azatriphenylenes, I₂, metal halides, preferably transition metal halides, metal oxides, preferably metal oxides comprising at least one transition metal or a metal from main group 3, and transition metal complexes, preferably complexes of Cu, Co, Ni, Pd and Pt with ligands containing at least one oxygen atom as binding site. Preference is further given to transition metal oxides as dopants, preferably oxides of rhenium, molybdenum and tungsten, more preferably Re₂O₇, MoO₃, WO₃ and ReO₃. Still further preference is given to complexes of bismuth in the (III) oxidation state, more particularly bismuth(III) complexes with electron-deficient ligands, more particularly carboxylate ligands.

The p-dopants are preferably in substantially homogeneous distribution in the p-doped layers. This can be achieved, for example, by co-evaporation of the p-dopant and the hole transport material matrix. The p-dopant is preferably present in a proportion of 1% to 10% in the p-doped layer.

Preferred p-dopants are especially the following compounds:

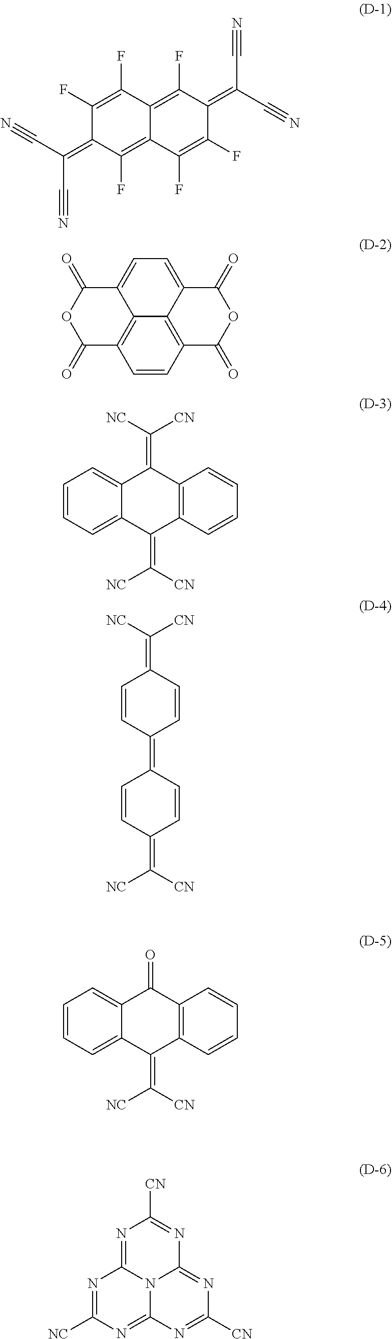

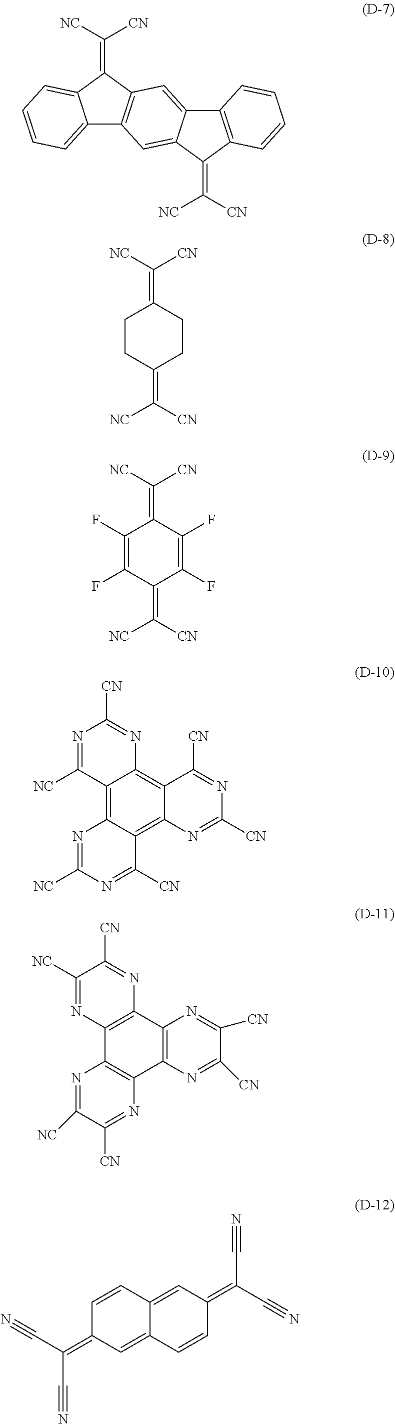

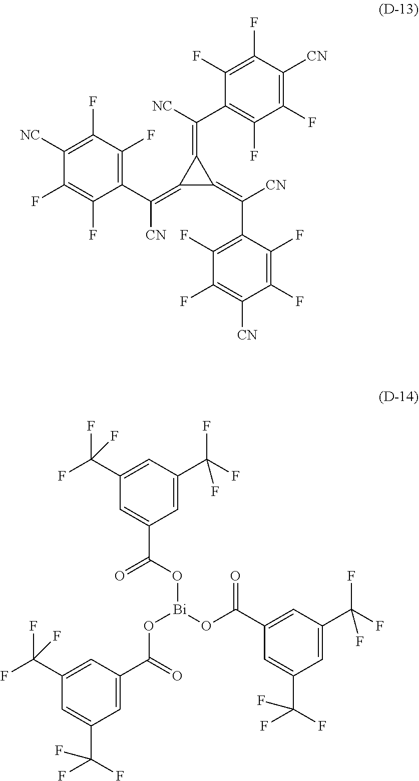

In a preferred embodiment, a hole injection layer that conforms to one of the following embodiments is present in the device: a) it contains a triarylamine and a p-dopant; or b) it contains a single electron-deficient material (electron acceptor). In a preferred embodiment of embodiment a), the triarylamine is a monotriarylamine, especially one of the preferred triarylamine derivatives mentioned further down. In a preferred embodiment of embodiment b), the electron-deficient material is a hexaazatriphenylene derivative as described in US 2007/0092755.

The compound of the formula (I) or (II) may be present in a hole injection layer, in a hole transport layer and/or in an electron blocker layer of the device. When the compound is present in a hole injection layer or in a hole transport layer, it has preferably been p-doped, meaning that it is in mixed form with a p-dopant, as described above, in the layer.

The compound of the formula (I) or (II) is preferably present in an electron blocker layer. In this case, it is preferably not p-doped. Further preferably, in this case, it is preferably in the form of a single compound in the layer without addition of a further compound.

In an alternative preferred embodiment, the compound of the formula (I) or (II) is used in an emitting layer as matrix material in combination with one or more emitting compounds, preferably phosphorescent emitting compounds. The phosphorescent emitting compounds here are preferably selected from red-phosphorescing and green-phosphorescing compounds.

The proportion of the matrix material in the emitting layer in this case is between 50.0% and 99.9% by volume, preferably between 80.0% and 99.5% by volume, and more preferably between 85.0% and 97.0% by volume.

Correspondingly, the proportion of the emitting compound is between 0.1% and 50.0% by volume, preferably between 0.5% and 20.0% by volume, and more preferably between 3.0% and 15.0% by volume.

An emitting layer of an organic electroluminescent device may also contain systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds of formula (I) or (II) are used as a component of mixed matrix systems, preferably for phosphorescent emitters. The mixed matrix systems preferably comprise two or three different matrix materials, more preferably two different matrix materials. Preferably, in this case, one of the two materials is a material having hole-transporting properties and the other material is a material having electron-transporting properties. It is further preferable when one of the materials is selected from compounds having a large energy differential between HOMO and LUMO (wide-bandgap materials). The compound of the formula (I) or (II) in a mixed matrix system is preferably the matrix material having hole-transporting properties. Correspondingly, when the compound of the formula (I) or (II) is used as matrix material for a phosphorescent emitter in the emitting layer of an OLED, a second matrix compound having electron-transporting properties is present in the emitting layer. The two different matrix materials may be present here in a ratio of 1:50 to 1:1, preferably 1:20 to 1:1, more preferably 1:10 to 1:1 and most preferably 1:4 to 1:1.

The desired electron-transporting and hole-transporting properties of the mixed matrix components may, however, also be combined mainly or entirely in a single mixed matrix component, in which case the further mixed matrix component(s) fulfil(s) other functions.

Preference is given to using the following material classes in the abovementioned layers of the device:

### Phosphorescent Emitters:

The term “phosphorescent emitters” typically encompasses compounds where the emission of light is effected through a spin-forbidden transition, for example a transition from an excited triplet state or a state having a higher spin quantum number, for example a quintet state.

Suitable phosphorescent emitters are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitters, compounds containing copper, molybdenum, tungsten, rhenium, ruthenium, osmium, rhodium, iridium, palladium, platinum, silver, gold or europium, especially compounds containing iridium, platinum or copper.

In the context of the present invention, all luminescent iridium, platinum or copper complexes are considered to be phosphorescent compounds.

In general, all phosphorescent complexes as used for phosphorescent OLEDs according to the prior art and as known to those skilled in the art in the field of organic electroluminescent devices are suitable for use in the devices of the invention. Further examples of suitable phosphorescent emitters are shown in the following table:

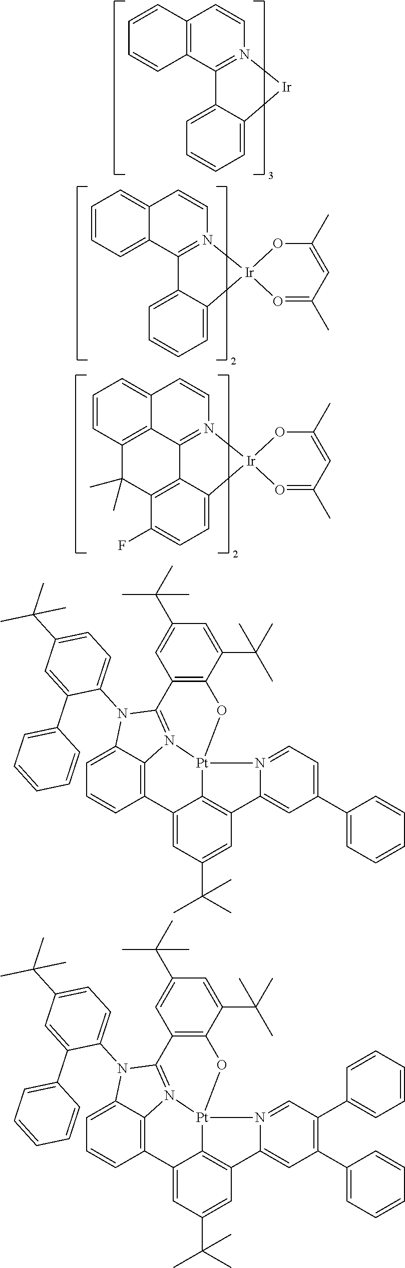

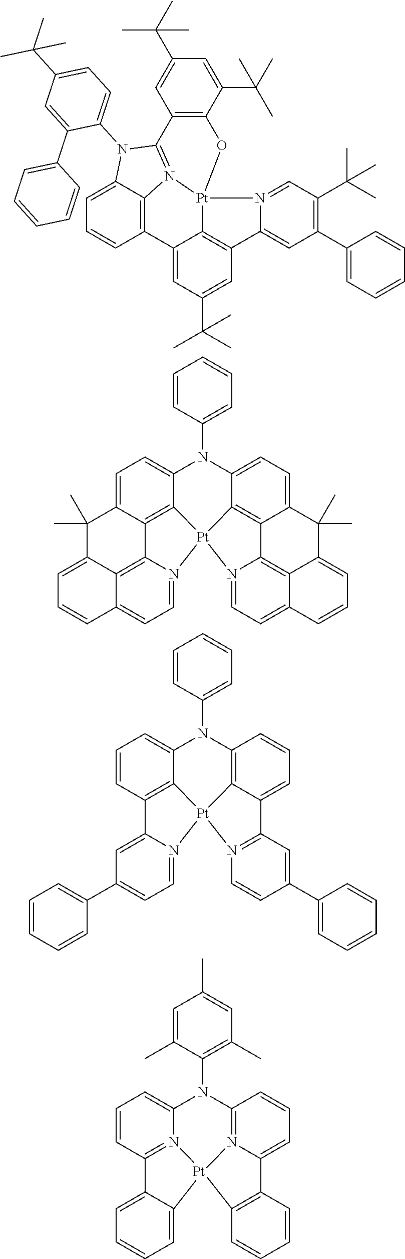

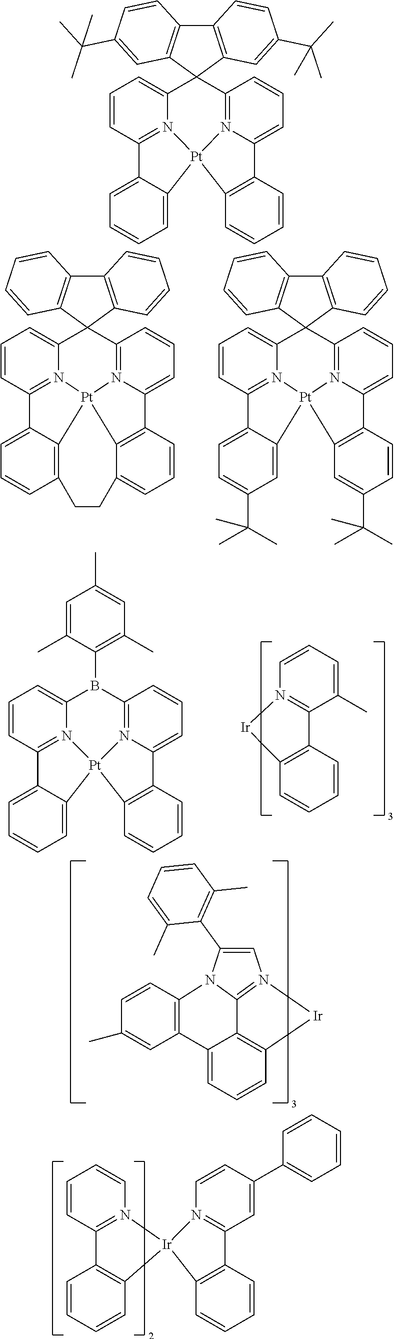

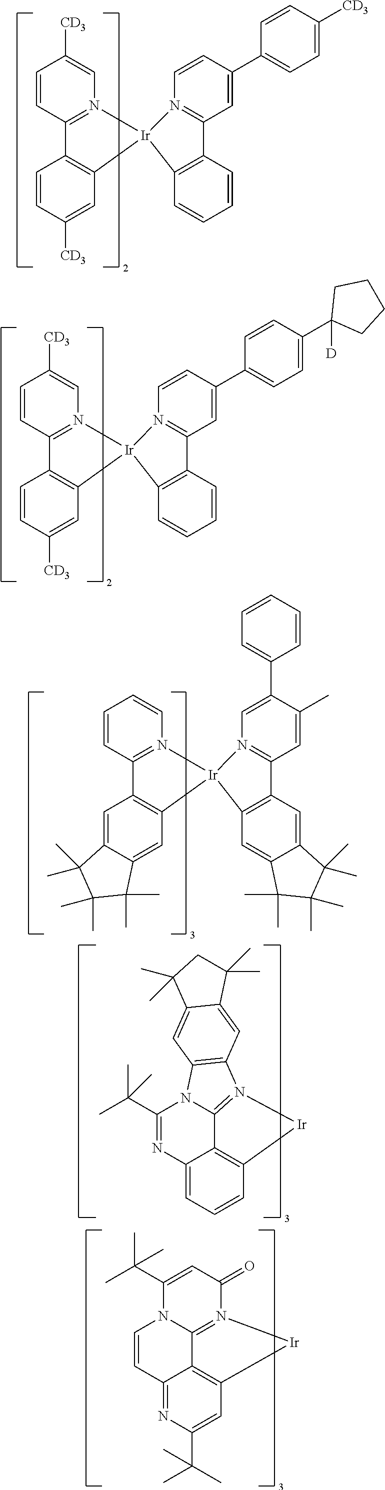

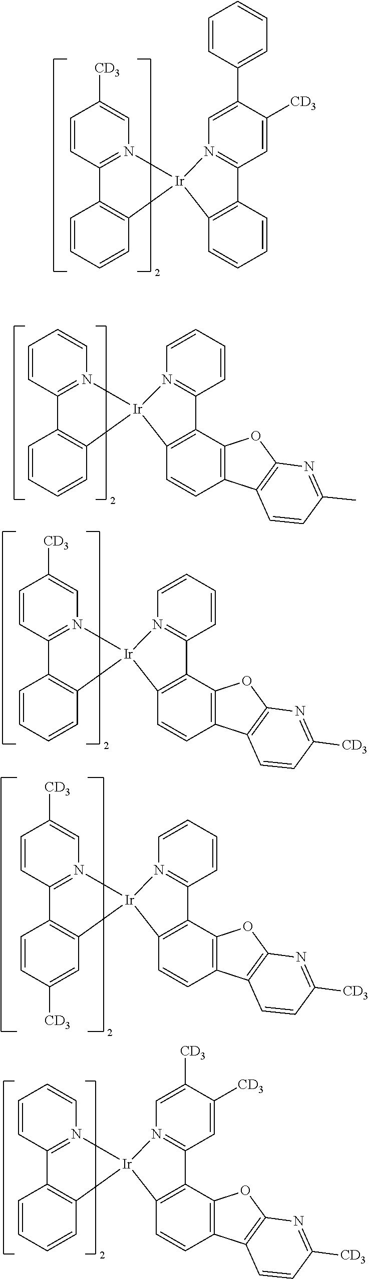

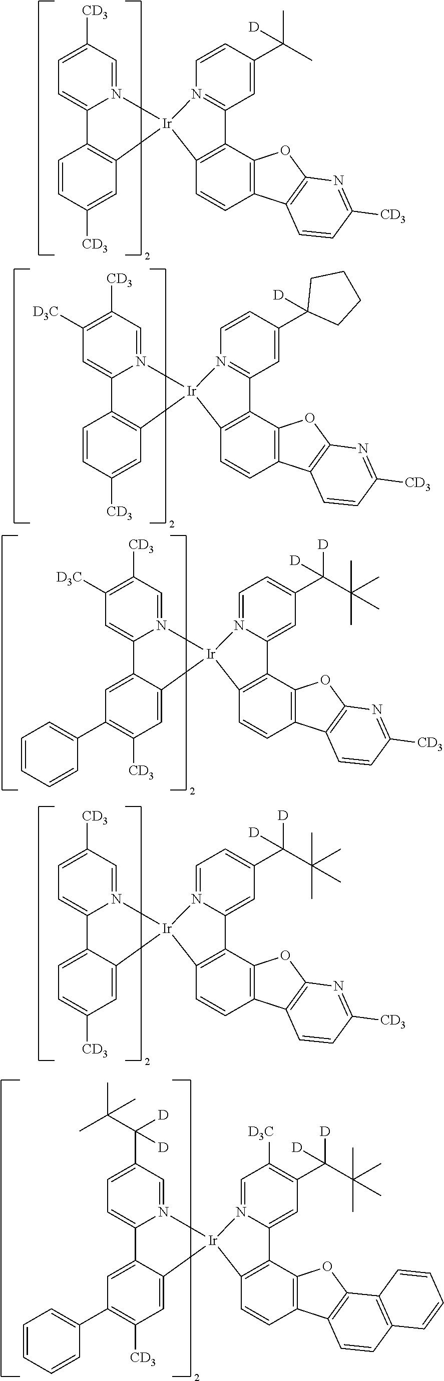

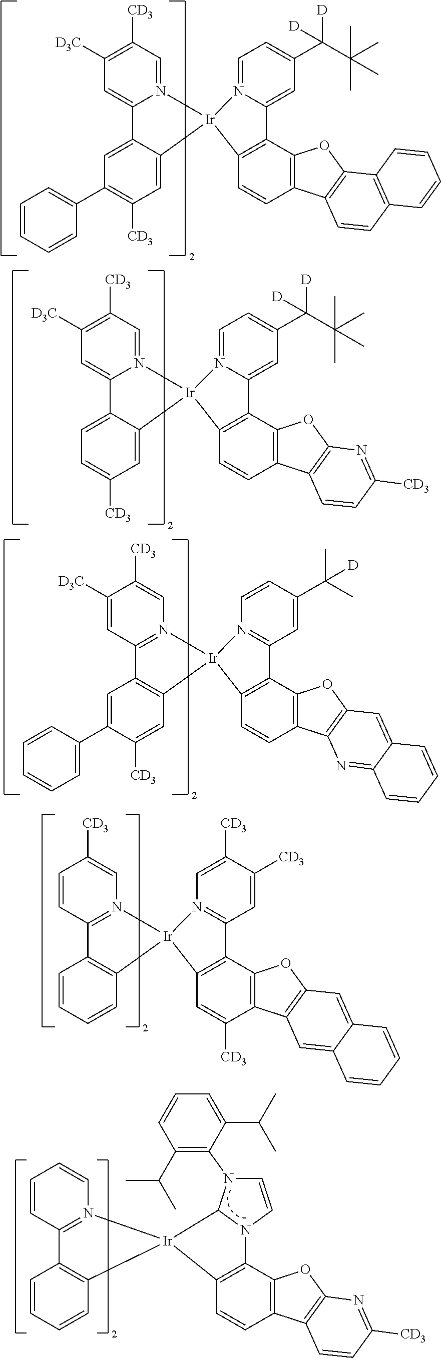

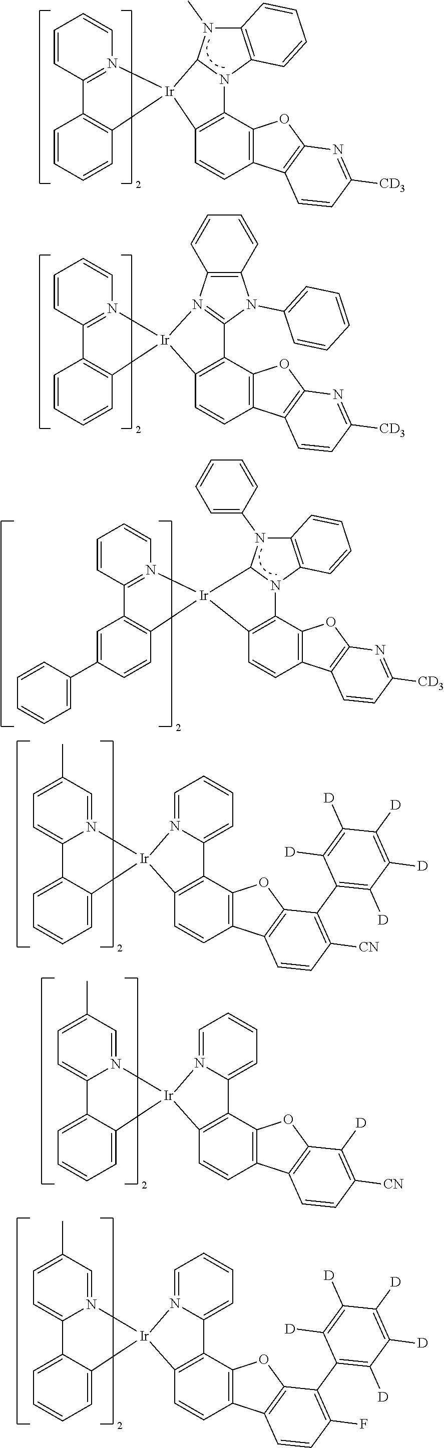

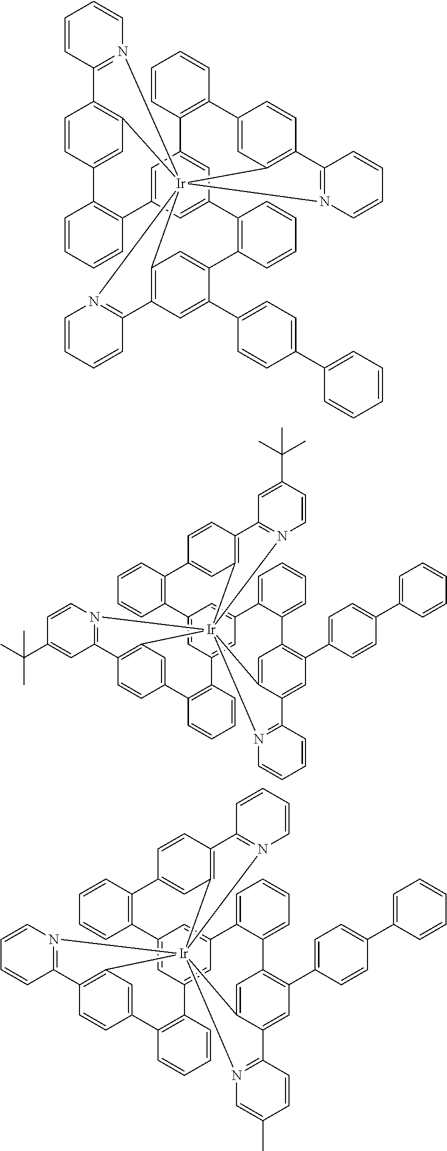

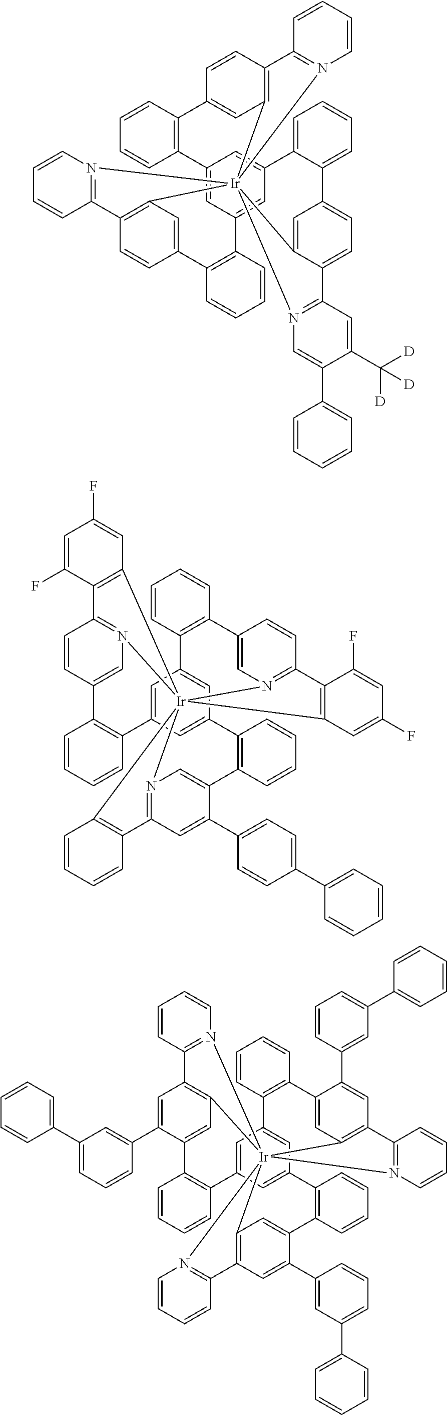

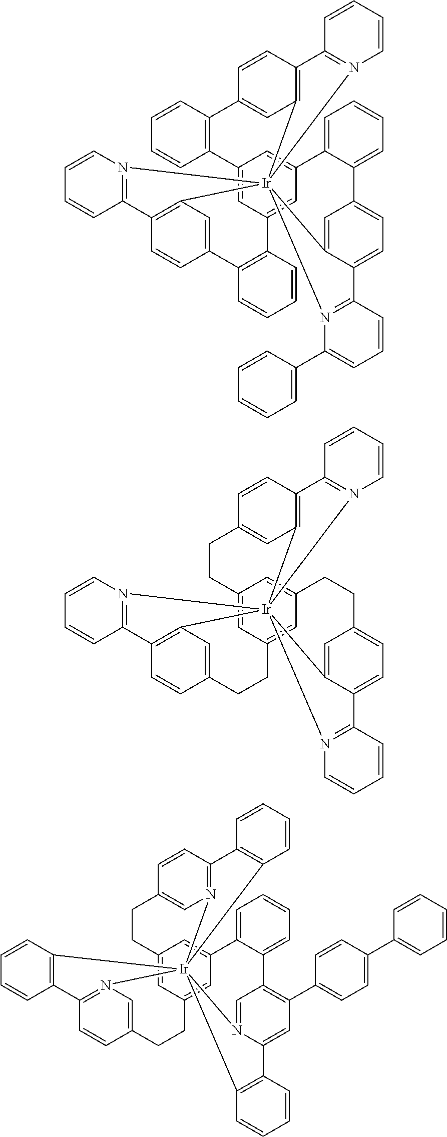

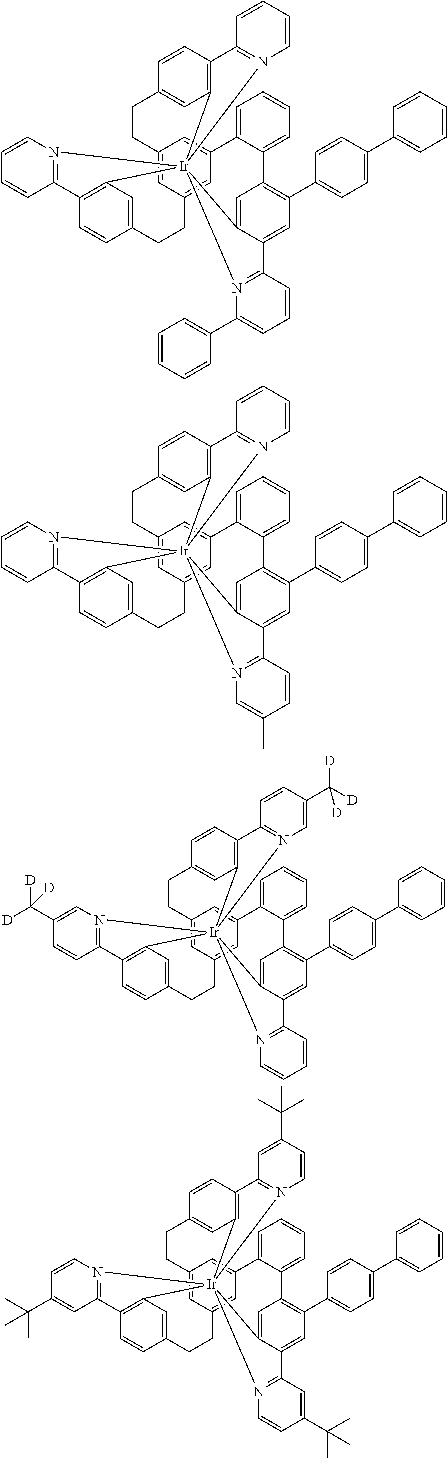

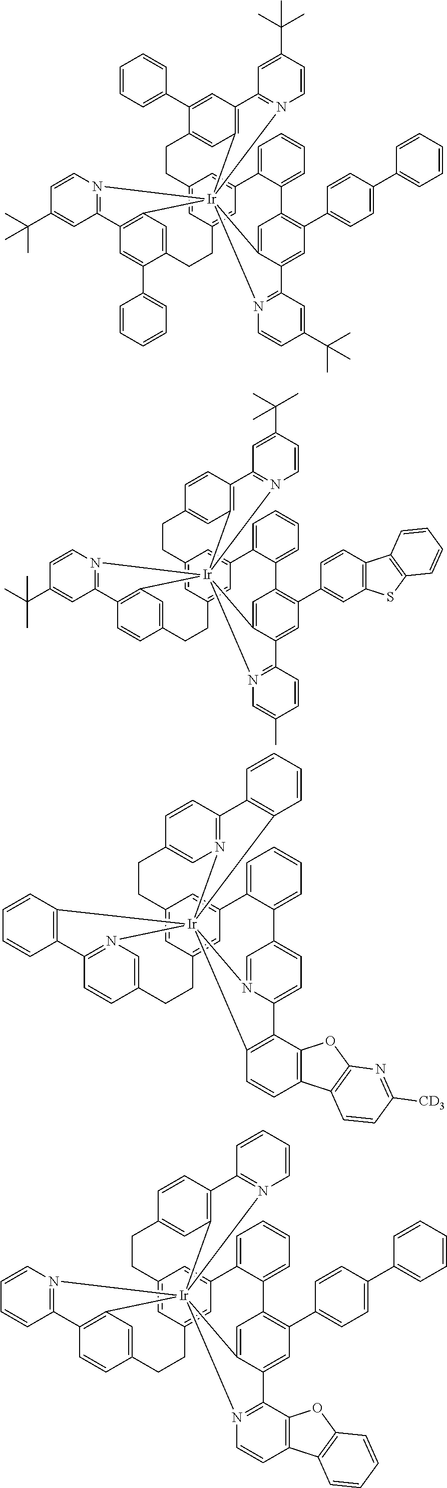

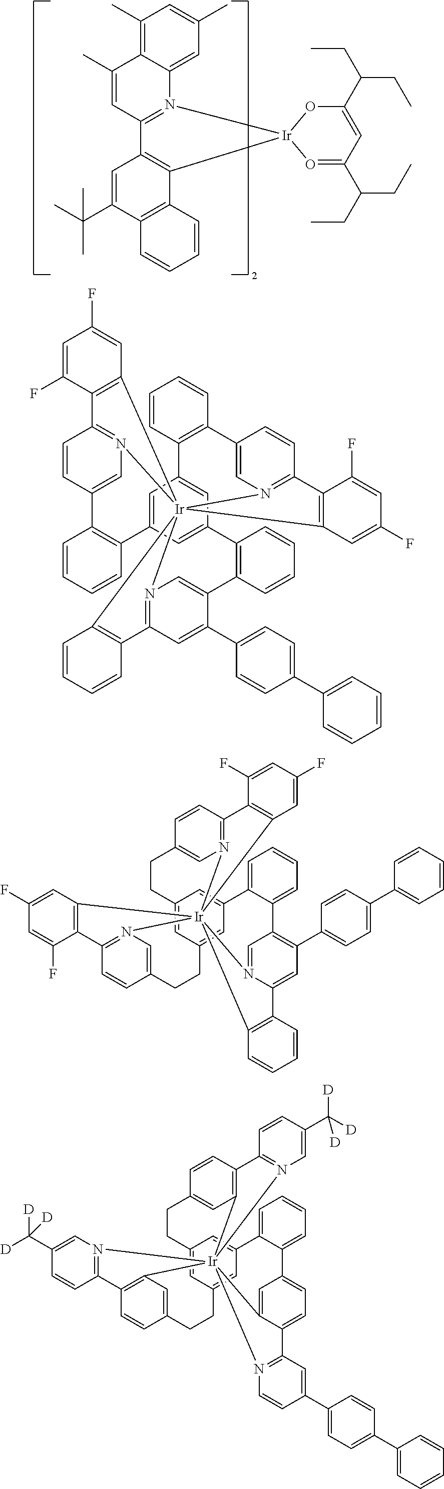

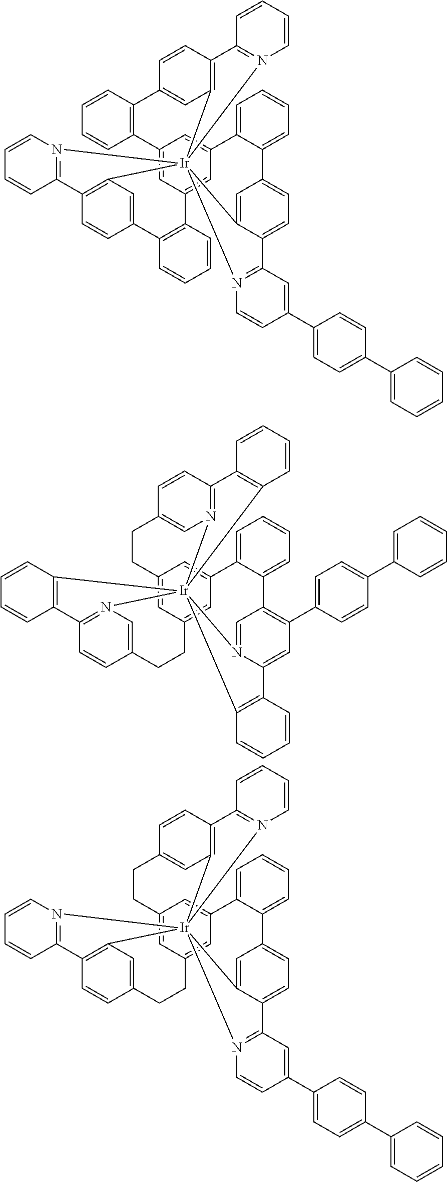

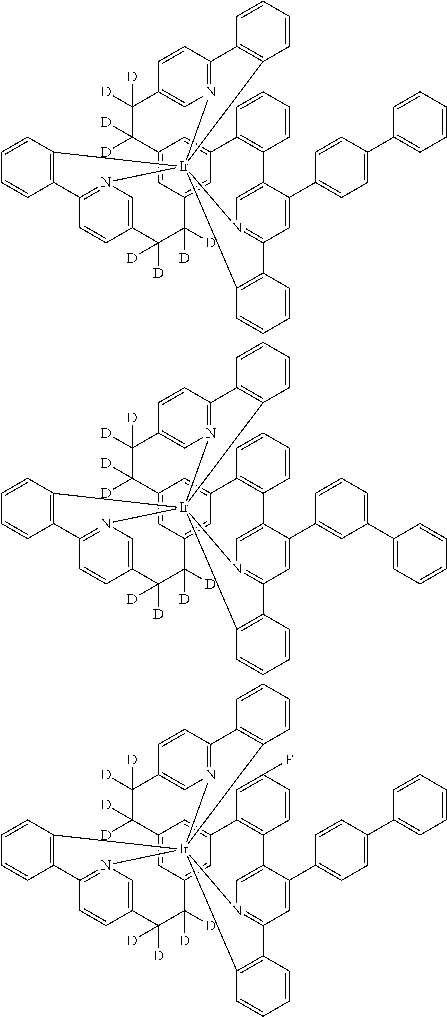

### Fluorescent Emitters:

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. What is meant by an aromatic anthraceneamine is a compound in which a diarylamino group is bonded directly to an anthracene group, preferably in the 9 position. What is meant by an aromatic anthracenediamine is a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 positions. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

### Matrix Materials for Fluorescent Emitters:

Preferred matrix materials for fluorescent emitters are selected from the classes of the oligoarylenes (e.g. 2,2′,7,7′-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

### Matrix Materials for Phosphorescent Emitters:

Preferred matrix materials for phosphorescent emitters are, as well as the compounds of the formula (I) or (II), aromatic ketones, aromatic phosphine oxides or aromatic sulfoxides or sulfones, triarylamines, carbazole derivatives, e.g. CBP (N,N-biscarbazolylbiphenyl), indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

### Electron-Transporting Materials:

Suitable electron-transporting materials are, for example, the compounds disclosed in Y. Shirota et al., Chem. Rev. 2007, 107 (4), 953-1010, or other materials used in these layers according to the prior art.

Materials used for the electron transport layer may be any materials that are used as electron transport materials in the electron transport layer according to the prior art. Especially suitable are aluminum complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

Preferred electron transport and electron injection materials are shown on pages 73-75 of WO2020/109434A1.

### Hole-Transporting Materials:

Further compounds which, in addition to the compounds of the formula (I) and (II), are preferably used in hole-transporting layers of the OLEDs of the invention are indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups.

Preferred hole-transporting compounds are shown in the table on pages 76-80 of WO2020/109434A1.

The compounds HT-1 to HT-35 below are particularly good suitability for use in a layer having hole transport function in an OLED. This is true not only of OLEDs according to the definitions and claims of the present application but also of OLEDs in general:

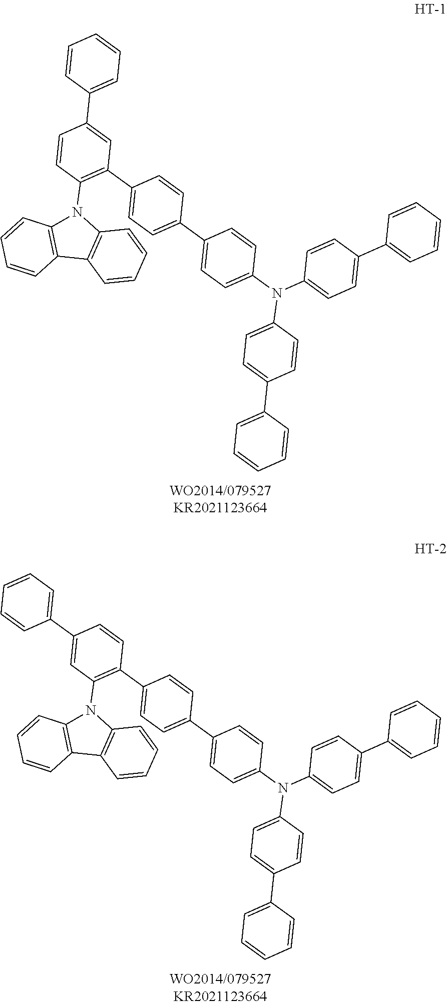 [SMILES: c1ccc(-c2ccc(N(c3ccc(-c4ccccc4)cc3)c3ccc(-c4ccc(-c5cc(-c6ccccc6)ccc5-n5c6ccccc6c6ccccc65)cc4)cc3)cc2)cc1 ; c1ccc(-c2ccc(N(c3ccc(-c4ccccc4)cc3)c3ccc(-c4ccc(-c5ccc(-c6ccccc6)cc5-n5c6ccccc6c6ccccc65)cc4)cc3)cc2)cc1]

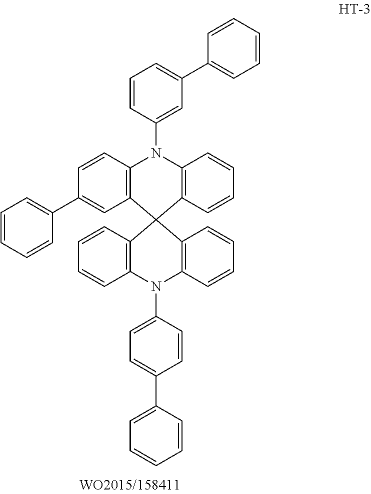 [SMILES: c1ccc(-c2ccc(N3c4ccccc4C4(c5ccccc53)c3ccccc3N(c3cccc(-c5ccccc5)c3)c3ccc(-c5ccccc5)cc34)cc2)cc1]

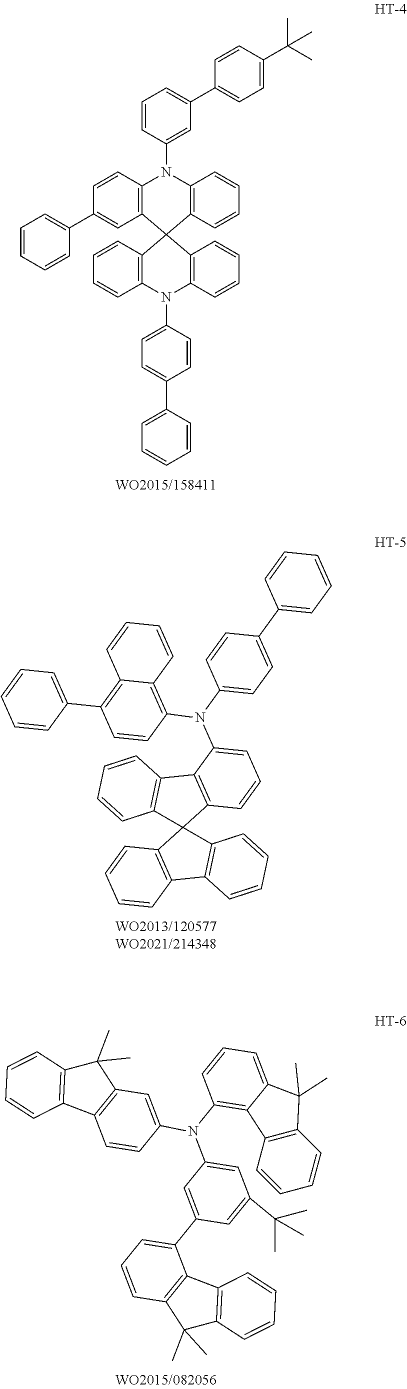 [SMILES: CC(C)(C)c1ccc(-c2cccc(N3c4ccccc4C4(c5ccccc5N(c5ccc(-c6ccccc6)cc5)c5ccccc54)c4cc(-c5ccccc5)ccc43)c2)cc1 ; c1ccc(-c2ccc(N(c3cccc4c3-c3ccccc3C43c4ccccc4-c4ccccc43)c3ccc(-c4ccccc4)c4ccccc34)cc2)cc1 ; CC(C)(C)c1cc(-c2cccc3c2-c2ccccc2C3(C)C)cc(N(c2ccc3c(c2)C(C)(C)c2ccccc2-3)c2cccc3c2-c2ccccc2C3(C)C)c1]

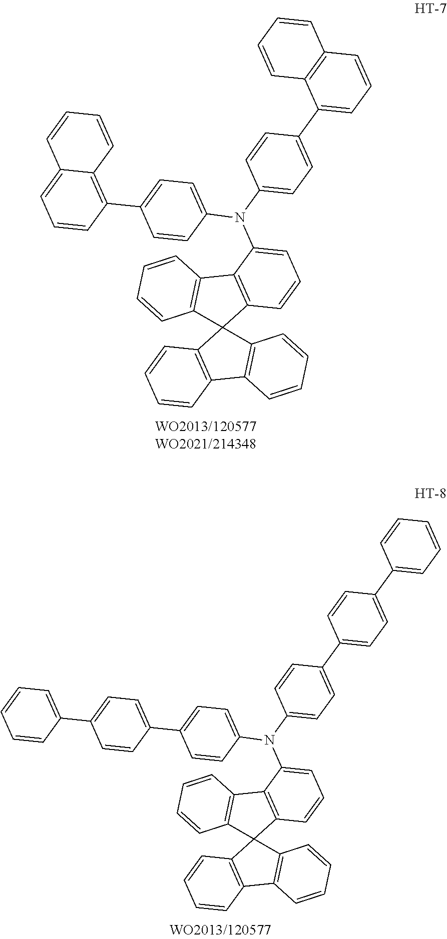 [SMILES: c1ccc2c(c1)-c1ccccc1C21c2ccccc2-c2c(N(c3ccc(-c4cccc5ccccc45)cc3)c3ccc(-c4cccc5ccccc45)cc3)cccc21 ; c1ccc(-c2ccc(-c3ccc(N(c4ccc(-c5ccc(-c6ccccc6)cc5)cc4)c4cccc5c4-c4ccccc4C54c5ccccc5-c5ccccc54)cc3)cc2)cc1]

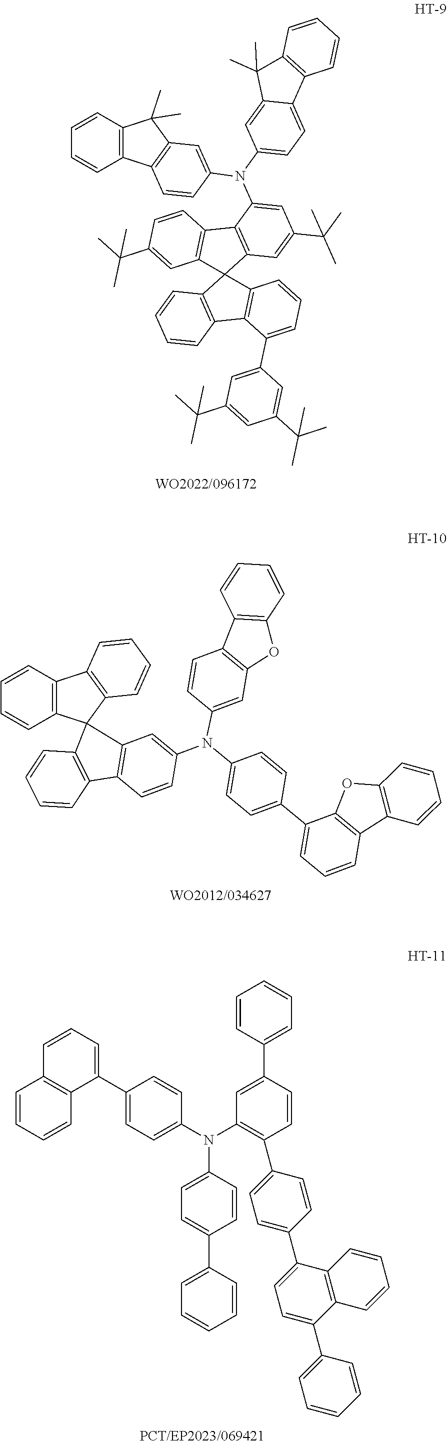 [SMILES: CC(C)(C)c1cc(-c2cccc3c2-c2ccccc2C32c3cc(C(C)(C)C)ccc3-c3c(N(c4ccc5c(c4)C(C)(C)c4ccccc4-5)c4ccc5c(c4)C(C)(C)c4ccccc4-5)cc(C(C)(C)C)cc32)cc(C(C)(C)C)c1 ; c1ccc2c(c1)-c1ccccc1C21c2ccccc2-c2ccc(N(c3ccc(-c4cccc5c4oc4ccccc45)cc3)c3ccc4c(c3)oc3ccccc34)cc21 ; c1ccc(-c2ccc(N(c3ccc(-c4cccc5ccccc45)cc3)c3cc(-c4ccccc4)ccc3-c3ccc(-c4ccc(-c5ccccc5)c5ccccc45)cc3)cc2)cc1]

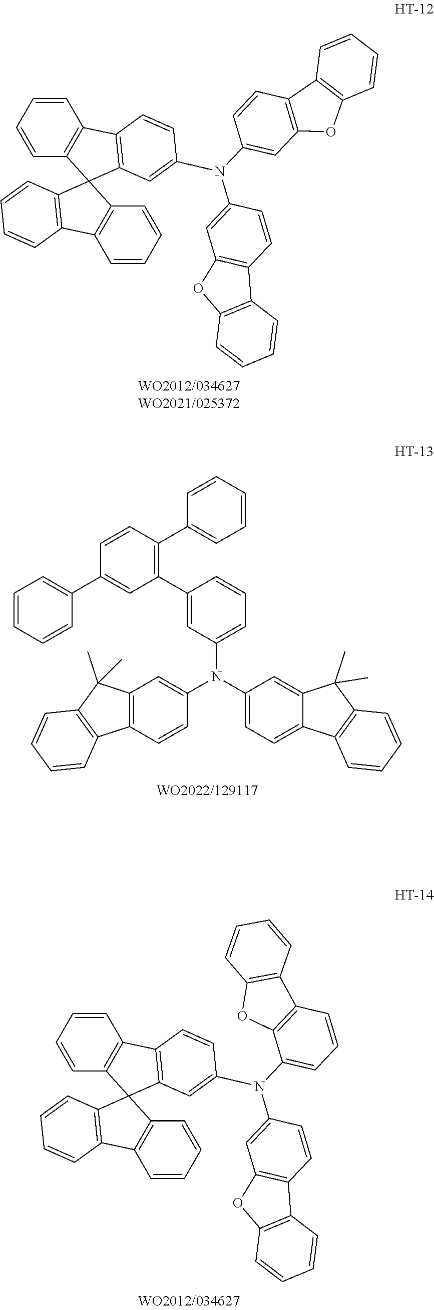 [SMILES: c1ccc2c(c1)-c1ccccc1C21c2ccccc2-c2ccc(N(c3ccc4c(c3)oc3ccccc34)c3ccc4c(c3)oc3ccccc34)cc21 ; CC1(C)c2ccccc2-c2ccc(N(c3cccc(-c4cc(-c5ccccc5)ccc4-c4ccccc4)c3)c3ccc4c(c3)C(C)(C)c3ccccc3-4)cc21 ; c1ccc2c(c1)-c1ccccc1C21c2ccccc2-c2ccc(N(c3ccc4c(c3)oc3ccccc34)c3cccc4c3oc3ccccc34)cc21]

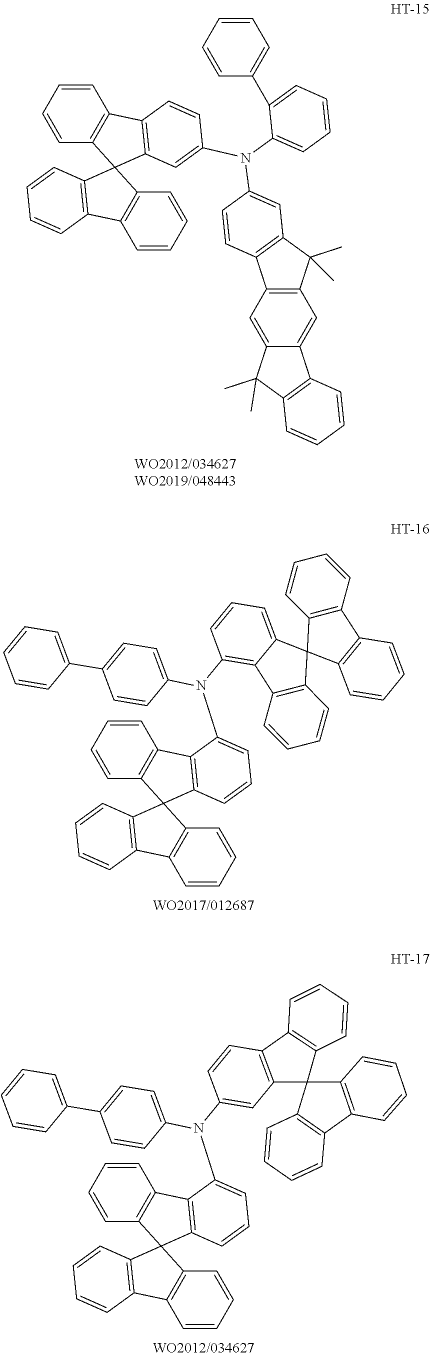 [SMILES: CC1(C)c2ccccc2-c2cc3c(cc21)-c1ccc(N(c2ccc4c(c2)C2(c5ccccc5-c5ccccc52)c2ccccc2-4)c2ccccc2-c2ccccc2)cc1C3(C)C ; c1ccc(-c2ccc(N(c3cccc4c3-c3ccccc3C43c4ccccc4-c4ccccc43)c3cccc4c3-c3ccccc3C43c4ccccc4-c4ccccc43)cc2)cc1 ; c1ccc(-c2ccc(N(c3ccc4c(c3)C3(c5ccccc5-c5ccccc53)c3ccccc3-4)c3cccc4c3-c3ccccc3C43c4ccccc4-c4ccccc43)cc2)cc1]

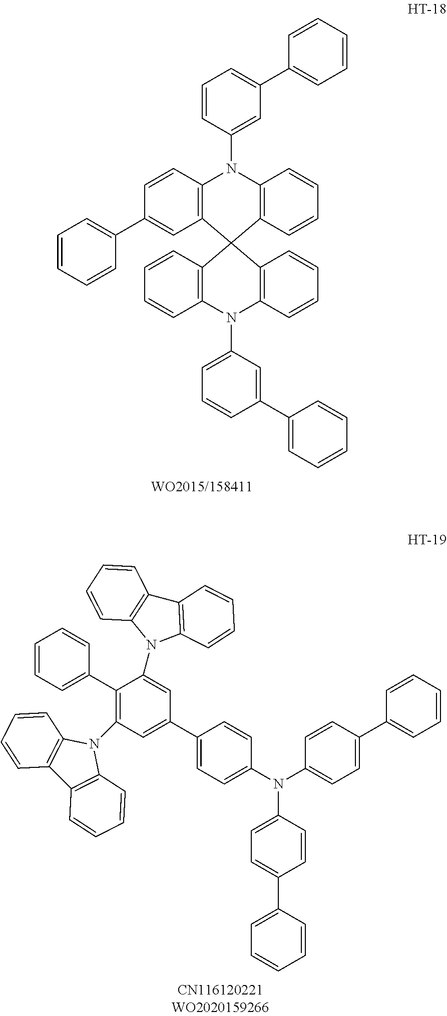 [SMILES: c1ccc(-c2cccc(N3c4ccccc4C4(c5ccccc53)c3ccccc3N(c3cccc(-c5ccccc5)c3)c3ccc(-c5ccccc5)cc34)c2)cc1 ; c1ccc(-c2ccc(N(c3ccc(-c4ccccc4)cc3)c3ccc(-c4cc(-n5c6ccccc6c6ccccc65)c(-c5ccccc5)c(-n5c6ccccc6c6ccccc65)c4)cc3)cc2)cc1]

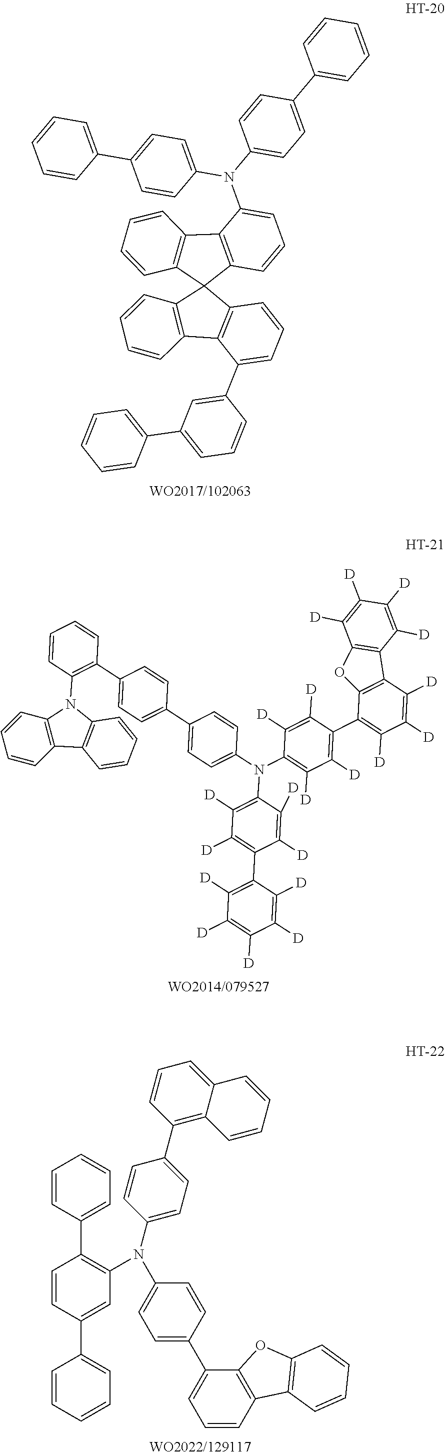 [SMILES: c1ccc(-c2ccc(N(c3ccc(-c4ccccc4)cc3)c3cccc4c3-c3ccccc3C43c4ccccc4-c4c(-c5cccc(-c6ccccc6)c5)cccc43)cc2)cc1 ; [2H]c1c([2H])c([2H])c(-c2c([2H])c([2H])c(N(c3ccc(-c4ccc(-c5ccccc5-n5c6ccccc6c6ccccc65)cc4)cc3)c3c([2H])c([2H])c(-c4c([2H])c([2H])c([2H])c5c4oc4c([2H])c([2H])c([2H])c([2H])c45)c([2H])c3[2H])c([2H])c2[2H])c([2H])c1[2H] ; c1ccc(-c2ccc(-c3ccccc3)c(N(c3ccc(-c4cccc5ccccc45)cc3)c3ccc(-c4cccc5c4oc4ccccc45)cc3)c2)cc1]

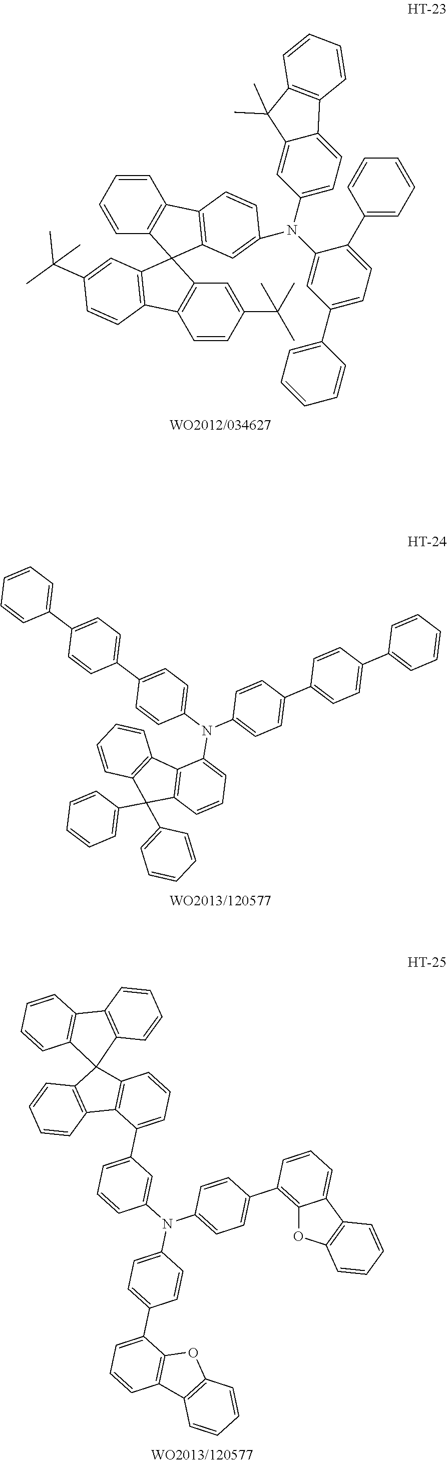 [SMILES: CC(C)(C)c1ccc2c(c1)C1(c3ccccc3-c3ccc(N(c4ccc5c(c4)C(C)(C)c4ccccc4-5)c4cc(-c5ccccc5)ccc4-c4ccccc4)cc31)c1cc(C(C)(C)C)ccc1-2 ; c1ccc(-c2ccc(-c3ccc(N(c4ccc(-c5ccc(-c6ccccc6)cc5)cc4)c4cccc5c4-c4ccccc4C5(c4ccccc4)c4ccccc4)cc3)cc2)cc1 ; c1cc(-c2cccc3c2-c2ccccc2C32c3ccccc3-c3ccccc32)cc(N(c2ccc(-c3cccc4c3oc3ccccc34)cc2)c2ccc(-c3cccc4c3oc3ccccc34)cc2)c1]

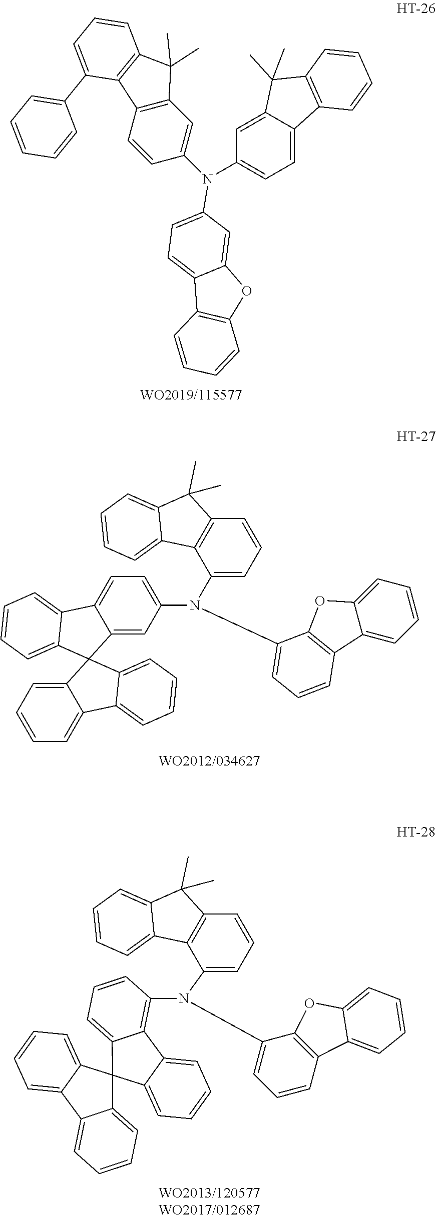 [SMILES: CC1(C)c2ccccc2-c2ccc(N(c3ccc4c(c3)C(C)(C)c3cccc(-c5ccccc5)c3-4)c3ccc4c(c3)oc3ccccc34)cc21 ; CC1(C)c2ccccc2-c2c(N(c3ccc4c(c3)C3(c5ccccc5-c5ccccc53)c3ccccc3-4)c3cccc4c3oc3ccccc34)cccc21 ; CC1(C)c2ccccc2-c2c(N(c3cccc4c3-c3ccccc3C43c4ccccc4-c4ccccc43)c3cccc4c3oc3ccccc34)cccc21]

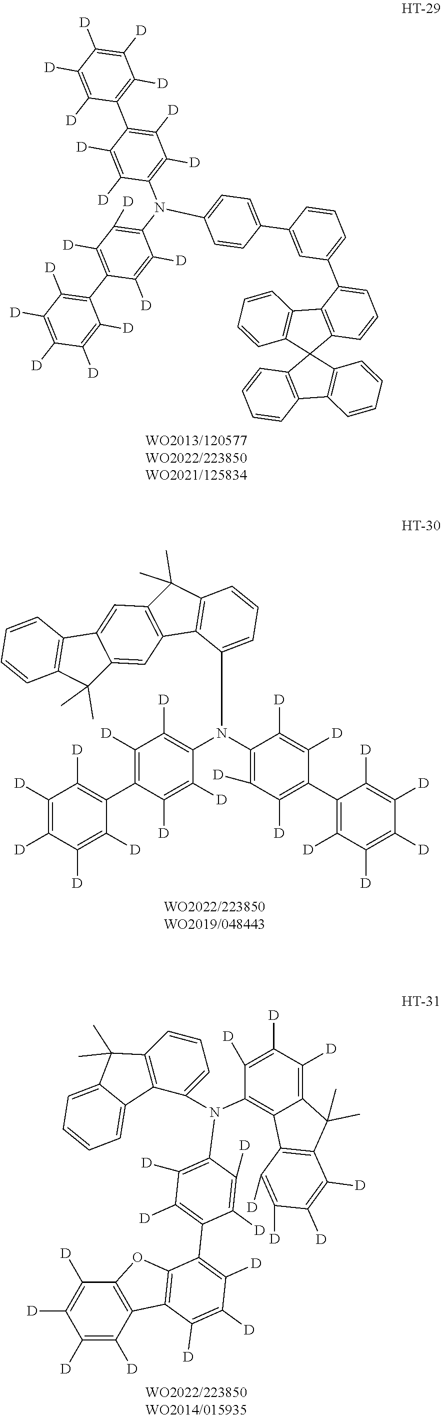

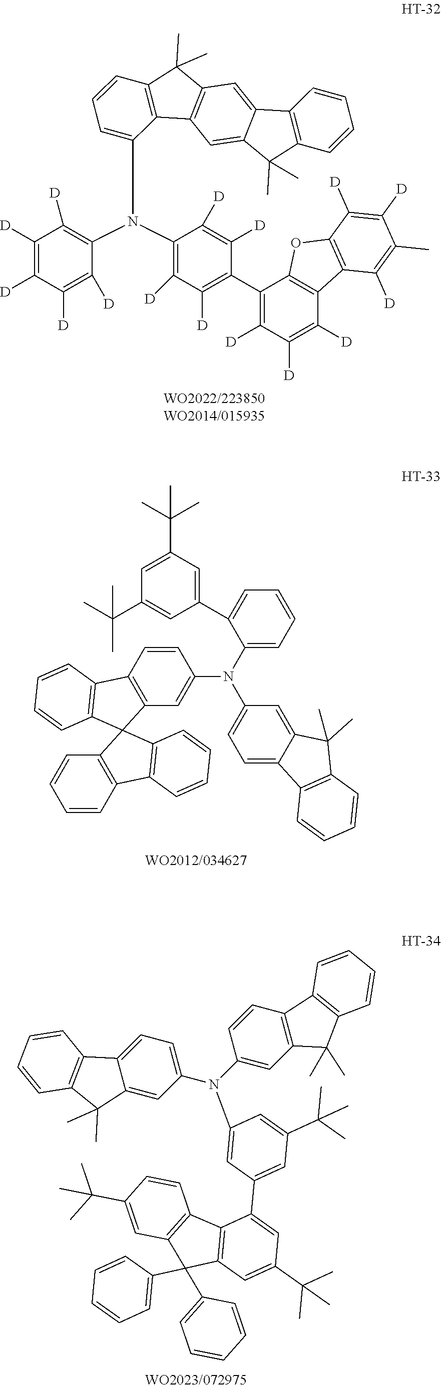 [SMILES: [2H]c1c([2H])c([2H])c(N(c2cccc3c2-c2cc4c(cc2C3(C)C)-c2ccccc2C4(C)C)c2c([2H])c([2H])c(-c3c([2H])c([2H])c([2H])c4c3oc3c([2H])c([2H])c(C)c([2H])c34)c([2H])c2[2H])c([2H])c1[2H] ; CC(C)(C)c1cc(-c2ccccc2N(c2ccc3c(c2)C(C)(C)c2ccccc2-3)c2ccc3c(c2)C2(c4ccccc4-c4ccccc42)c2ccccc2-3)cc(C(C)(C)C)c1 ; CC(C)(C)c1cc(-c2cc(C(C)(C)C)cc3c2-c2ccc(C(C)(C)C)cc2C3(c2ccccc2)c2ccccc2)cc(N(c2ccc3c(c2)C(C)(C)c2ccccc2-3)c2ccc3c(c2)C(C)(C)c2ccccc2-3)c1]

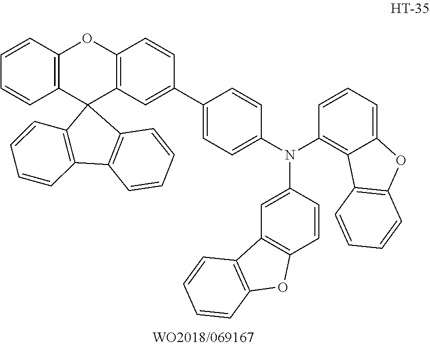 [SMILES: c1ccc2c(c1)Oc1ccc(-c3ccc(N(c4ccc5oc6ccccc6c5c4)c4cccc5oc6ccccc6c45)cc3)cc1C21c2ccccc2-c2ccccc21]

Compounds HT-1 to HT-35 may generally used in any hole transport layers of OLEDs. The term “hole transport layer” here means any layer of an OLED between anode and emitting layer. The term “OLED” is not especially restricted and applies to all OLEDs, especially OLED structures that were customary at the filing date of the present application.

The compounds HT-1 to HT-35 may be prepared by methods disclosed in the application texts listed in the above tables under the respective compounds HT-1 to HT-35. The teaching relating to the use of the compounds and the processes for preparing the compounds in the abovementioned application texts are hereby explicitly incorporated into the present disclosure by reference.

Compounds HT-1 to HT-35 have excellent properties when used in OLEDs, especially excellent lifetime and efficiency. This is the case especially when they are used in a hole transport layer of the OLED.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the outcoupling of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

In a preferred embodiment, the electronic device is characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (I) or (II) are needed. High solubility can be achieved by suitable substitution of the compounds.

It is further preferable that an electronic device of the invention is produced by applying one or more layers from solution and one or more layers by a sublimation method.

After application of the layers, according to the use, the device is structured, contact-connected and finally sealed, in order to rule out damaging effects of water and air.

According to the invention, the electronic devices comprising one or more compounds of formula (I) or (II) can be used in displays, as light sources in lighting applications and as light sources in medical and/or cosmetic applications.

### EXAMPLES

### A) Synthesis Examples

### 1) Synthesis of Compound 1a

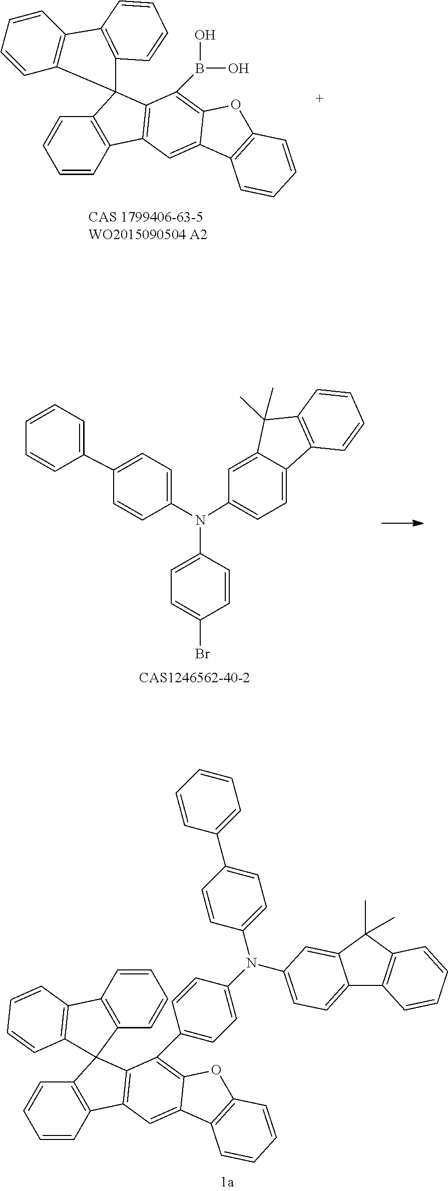 [SMILES: OB(O)c1c2c(cc3c1oc1ccccc13)-c1ccccc1C21c2ccccc2-c2ccccc21 ; CC1(C)c2ccccc2-c2ccc(N(c3ccc(Br)cc3)c3ccc(-c4ccccc4)cc3)cc21 ; CC1(C)c2ccccc2-c2ccc(N(c3ccc(-c4ccccc4)cc3)c3ccc(-c4c5c(cc6c4oc4ccccc46)-c4ccccc4C54c5ccccc5-c5ccccc54)cc3)cc21]

33.4 g (63 mmol) of CAS 1799406-63-5, 32.4 (63 mmol), 21.1 g (94 mmol) of potassium phosphate monohydrate and 1.6 g (1.9 mmol) of XPhos Palladacycle Gen.3 are dissolved in 60 ml of THF/water (4:1) and stirred at 60° C. for 16 hours. The reaction mixture is then concentrated on a rotary evaporator and the residue is dissolved with dichloromethane. The organic phase is washed twice with water, and the aqueous phases are extracted twice with dichloromethane. The organic phases are combined and filtered dried over sodium sulfate and concentrated to dryness on a rotary evaporator. The residue is repeatedly subjected to hot extraction over aluminum oxide (toluene/heptane 1:1) and crystallized to an HPLC purity of >99.9%. Finally, the product, after sublimation (10⁻⁶ bar, 325° C.), is obtained in solid form.

Yield: 25.9 g (30.7 mmol; 49%)

The following compounds are prepared in an analogous manner:

| Ex. | Reactant 1 | Reactant 2 | Product |
|---|---|---|---|
| 1b | CAS 1799406-63-5 | CAS 499128-71-1 | 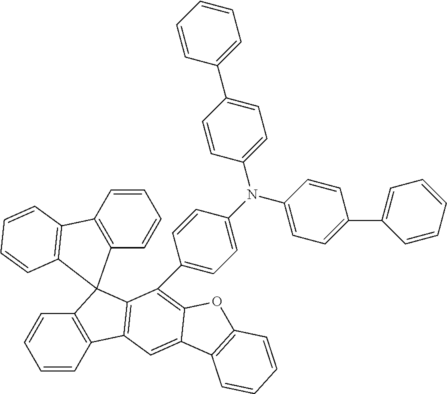 [SMILES: c1ccc(-c2ccc(N(c3ccc(-c4ccccc4)cc3)c3ccc(-c4c5c(cc6c4oc4ccccc46)-c4ccccc4C54c5ccccc5-c5ccccc54)cc3)cc2)cc1] |
| 1c | CAS 1799406-63-5 | CAS 1443130-62-8 | 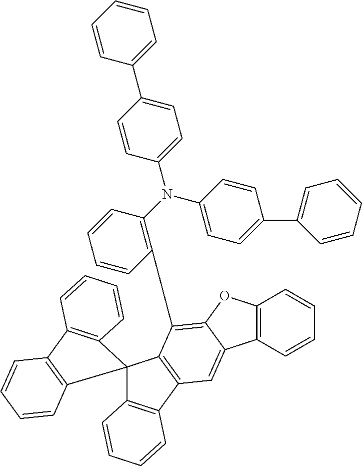 [SMILES: c1ccc(-c2ccc(N(c3ccc(-c4ccccc4)cc3)c3ccccc3-c3c4c(cc5c3oc3ccccc35)-c3ccccc3C43c4ccccc4-c4ccccc43)cc2)cc1] |
| 1d | CAS 1799406-63-5 | CAS 1160294-67-6 | 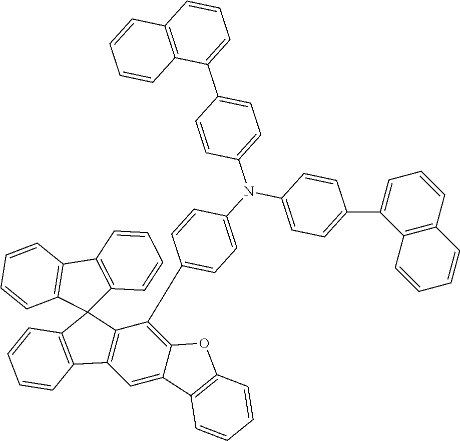 [SMILES: c1ccc2c(c1)-c1ccccc1C21c2ccccc2-c2cc3c(oc4ccccc43)c(-c3ccc(N(c4ccc(-c5cccc6ccccc56)cc4)c4ccc(-c5cccc6ccccc56)cc4)cc3)c21] |
| 1e | Int-4a | CAS 499128-71-1 | 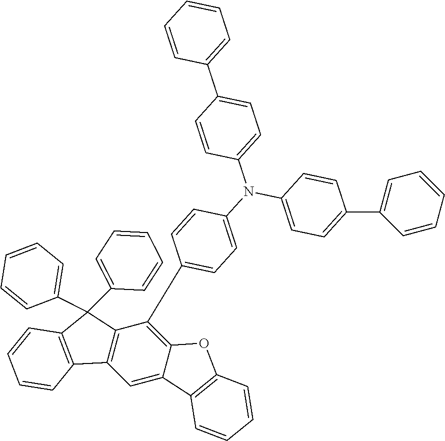 [SMILES: c1ccc(-c2ccc(N(c3ccc(-c4ccccc4)cc3)c3ccc(-c4c5c(cc6c4oc4ccccc46)-c4ccccc4C5(c4ccccc4)c4ccccc4)cc3)cc2)cc1] |
| 1f | CAS 1799406-63-5 | CAS 1609381-11-4 | 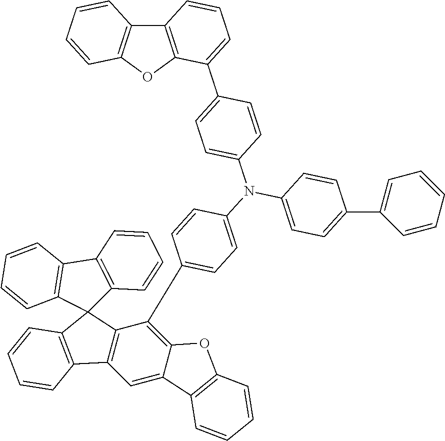 [SMILES: c1ccc(-c2ccc(N(c3ccc(-c4cccc5c4oc4ccccc45)cc3)c3ccc(-c4c5c(cc6c4oc4ccccc46)-c4ccccc4C54c5ccccc5-c5ccccc54)cc3)cc2)cc1] |
| Int-1a | CAS 1799406-63-5 | 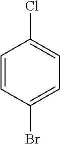 | 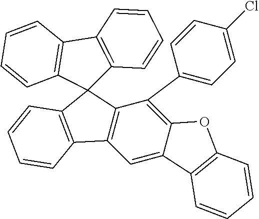 [SMILES: Clc1ccc(-c2c3c(cc4c2oc2ccccc24)-c2ccccc2C32c3ccccc3-c3ccccc32)cc1] |
| | | CAS 106-39-8 | |
| Int-1b | CAS 1799406-63-5 | 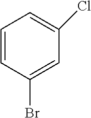 | 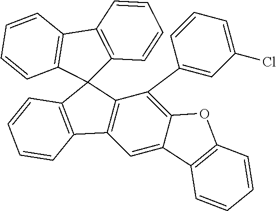 [SMILES: Clc1cccc(-c2c3c(cc4c2oc2ccccc24)-c2ccccc2C32c3ccccc3-c3ccccc32)c1] |
| | | CAS 108-37-2 | |
| Int-1c | Int-2 | 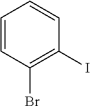 | 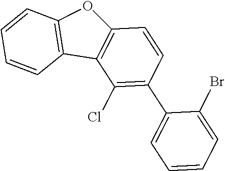 [SMILES: Clc1c(-c2ccccc2Br)ccc2oc3ccccc3c12] |
| | | CAS 583-55-1 | |

### 2) Synthesis of Int-2

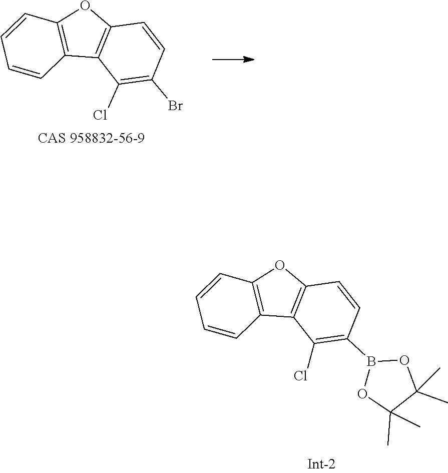 [SMILES: Clc1c(Br)ccc2oc3ccccc3c12 ; CC1(C)OB(c2ccc3oc4ccccc4c3c2Cl)OC1(C)C]

The synthesis of Int-1 is analogous to the synthesis of CAS 2459761-30-7 described in WO 2020/159333 A1.

### 3) Synthesis of Int-3a

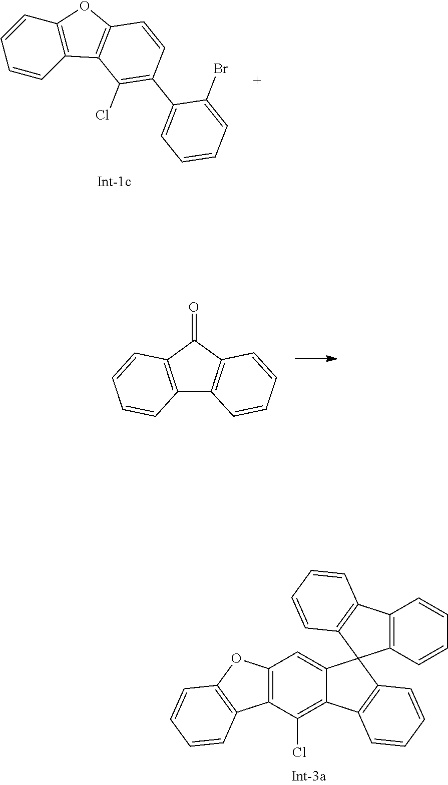 [SMILES: Clc1c(-c2ccccc2Br)ccc2oc3ccccc3c12 ; O=C1c2ccccc2-c2ccccc21 ; Clc1c2c(cc3oc4ccccc4c13)C1(c3ccccc3-c3ccccc31)c1ccccc1-2]

The synthesis of Int-3a is analogous to the two-stage synthesis of Int-7, described in WO 2015/022051 A1 (cf. page 85 ff.).

The following compounds are prepared in an analogous manner:

| Ex. | SM 1 | SM 2 | Product |
|---|---|---|---|
| Int-3b | 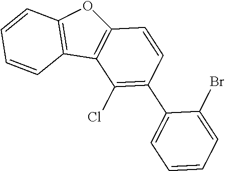 [SMILES: Clc1c(-c2ccccc2Br)ccc2oc3ccccc3c12] | 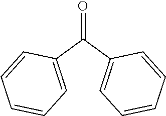 [SMILES: O=C(c1ccccc1)c1ccccc1] | 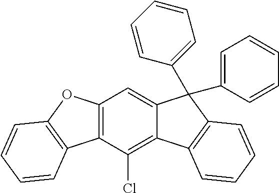 [SMILES: Clc1c2c(cc3oc4ccccc4c13)C(c1ccccc1)(c1ccccc1)c1ccccc1-2] |
| | Int-1c | | |
| Int-3c* | 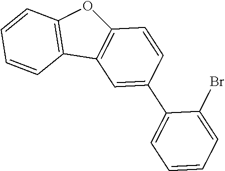 [SMILES: Brc1ccccc1-c1ccc2oc3ccccc3c2c1] | 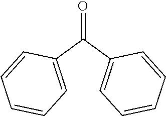 [SMILES: O=C(c1ccccc1)c1ccccc1] | 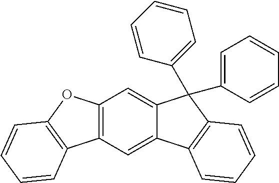 [SMILES: c1ccc(C2(c3ccccc3)c3ccccc3-c3cc4c(cc32)oc2ccccc24)cc1] |
| | CAS 1659313-52-6 | | |

*this reaction forms an isomer mixture that can be separated by chromatography.

### 4) Synthesis of Int-4a

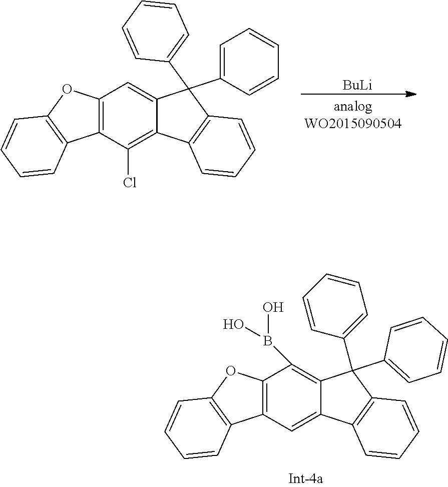

The boronic acid is prepared analogously to the synthesis of the compound CAS 1799406-63-5, described in WO 2015/090504, proceeding from Int-3c.

The following compounds are synthesized in an analogous manner:

| | | | |
|---|---|---|---|
| Int-4b | 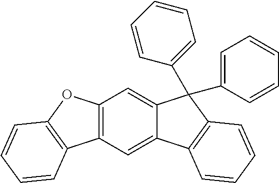 [SMILES: c1ccc(C2(c3ccccc3)c3ccccc3-c3cc4c(cc32)oc2ccccc24)cc1] | 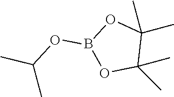 | 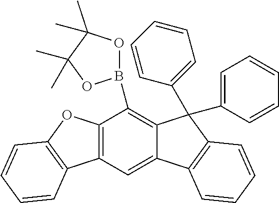 [SMILES: CC1(C)OB(c2c3c(cc4c2oc2ccccc24)-c2ccccc2C3(c2ccccc2)c2ccccc2)OC1(C)C] |
| Int-4c | 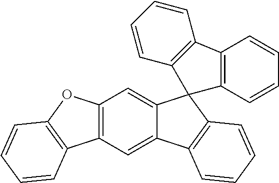 [SMILES: c1ccc2c(c1)-c1ccccc1C21c2ccccc2-c2cc3c(cc21)oc1ccccc13] | 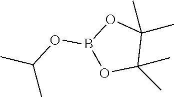 | 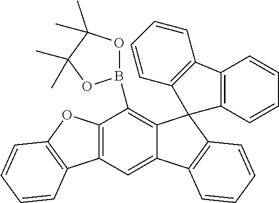 [SMILES: CC1(C)OB(c2c3c(cc4c2oc2ccccc24)-c2ccccc2C32c3ccccc3-c3ccccc32)OC1(C)C] |
| Int-4d | 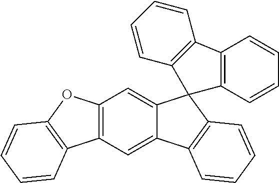 [SMILES: c1ccc2c(c1)-c1ccccc1C21c2ccccc2-c2cc3c(cc21)oc1ccccc13] | 1,2-dibromoethane | 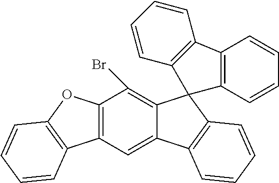 [SMILES: Brc1c2c(cc3c1oc1ccccc13)-c1ccccc1C21c2ccccc2-c2ccccc21] |
| Int-4e | 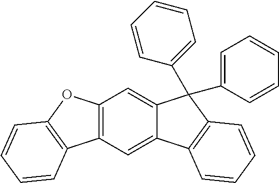 [SMILES: c1ccc(C2(c3ccccc3)c3ccccc3-c3cc4c(cc32)oc2ccccc24)cc1] | 1,2-dibromoethane | 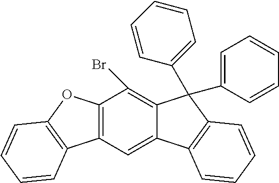 [SMILES: Brc1c2c(cc3c1oc1ccccc13)-c1ccccc1C2(c1ccccc1)c1ccccc1] |

### 5) Synthesis of Reference Compound 2a

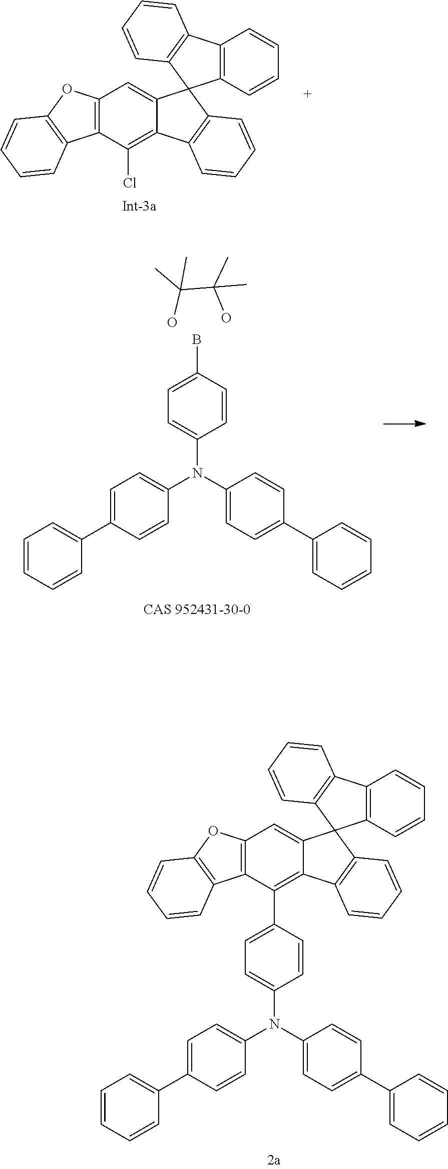

15.4 g (35 mmol) of Int-4, 8.3 g (35 mmol) of CAS 952431-30-0 and 10.6 g (70 mmol) of cesium fluoride are suspended in 400 ml of dioxane. 1.02 g (1.40 mmol) of bis(tricyclohexylphosphine) palladium dichloride is added to this suspension, and the reaction mixture is refluxed for 18 hours. Then the reaction mixture is left to cool to room temperature, and the organic phase is washed three times with 100 ml of water and concentrated to dryness on a rotary evaporator. The residue is taken up in toluene and filtered through silica gel, and the residue is purified by recrystallisation from toluene/heptane to an HPLC purity of >99.9% and finally sublimed under high vacuum. Yield: 10.4 g (13 mmol; 37% of theory).

The following compound is prepared analogously:

| Ref. compd. | Reactant 1 | Reactant 2 | Product |
|---|---|---|---|
| 2b | Int-3a | CAS 1347758-80-8 | 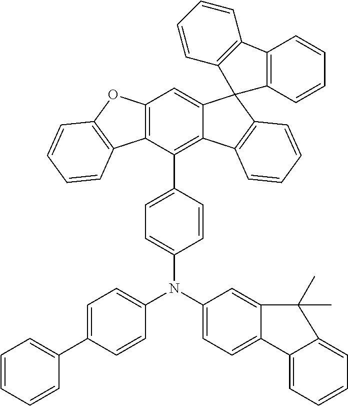 [SMILES: CC1(C)c2ccccc2-c2ccc(N(c3ccc(-c4ccccc4)cc3)c3ccc(-c4c5c(cc6oc7ccccc7c46)C4(c6ccccc6-c6ccccc64)c4ccccc4-5)cc3)cc21] |

### 6) Synthesis of Compound 3a

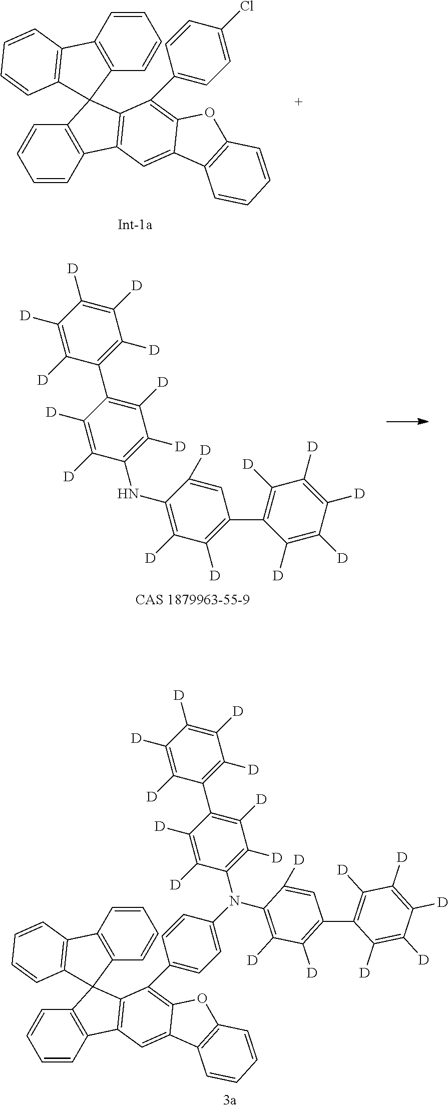 [SMILES: Clc1ccc(-c2c3c(cc4c2oc2ccccc24)-c2ccccc2C32c3ccccc3-c3ccccc32)cc1 ; [2H]c1cc(-c2c([2H])c([2H])c([2H])c([2H])c2[2H])c([2H])c([2H])c1Nc1c([2H])c([2H])c(-c2c([2H])c([2H])c([2H])c([2H])c2[2H])c([2H])c1[2H] ; [2H]c1cc(-c2c([2H])c([2H])c([2H])c([2H])c2[2H])c([2H])c([2H])c1N(c1ccc(-c2c3c(cc4c2oc2ccccc24)-c2ccccc2C32c3ccccc3-c3ccccc32)cc1)c1c([2H])c([2H])c(-c2c([2H])c([2H])c([2H])c([2H])c2[2H])c([2H])c1[2H]]

13.4 g (26 mmol) of Int-1a, 8.83 g (26 mmol) of CAS 1879963-55-9, 3.5 g (37 mmol) of sodium tert-butoxide and 616 mg (0.73 mmol) of XPhos Pd Gen3 are suspended in 400 ml of toluene and stirred at 100° C. for 16 hours. On completion of conversion, the reaction mixture is allowed to cool down to room temperature and filtered through aluminum oxide and washed with toluene. After the solvents have been removed, the crude product is dissolved in toluene/heptane 1:1 and filtered through silica gel. Further purification is effected by repeated crystallization from heptane/toluene up to an HPLC purity of >99.9%. Finally, the product, after two sublimations under high vacuum, is obtained in solid form.

Yield: 6.7 g (9 mmol; 35%)

The following compounds are prepared in an analogous manner:

| Ex. | Amine | Reactant | Product |
|---|---|---|---|
| 3b | CAS 1879963-55-9 | Int-1b | 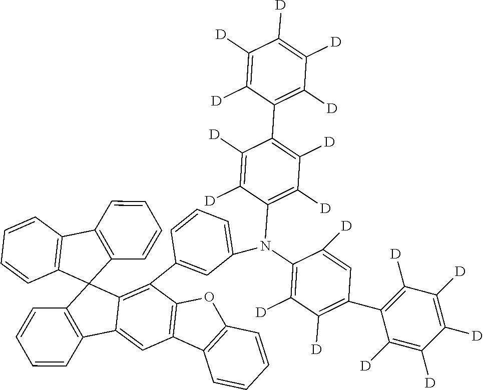 [SMILES: [2H]c1cc(-c2c([2H])c([2H])c([2H])c([2H])c2[2H])c([2H])c([2H])c1N(c1cccc(-c2c3c(cc4c2oc2ccccc24)-c2ccccc2C32c3ccccc3-c3ccccc32)c1)c1c([2H])c([2H])c(-c2c([2H])c([2H])c([2H])c([2H])c2[2H])c([2H])c1[2H]] |
| 3c | CAS 955959-89-4 | Int-1a | 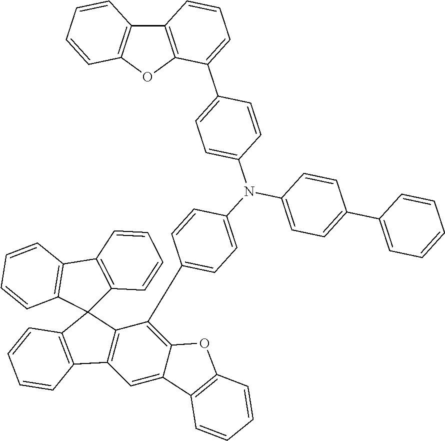 [SMILES: c1ccc(-c2ccc(N(c3ccc(-c4cccc5c4oc4ccccc45)cc3)c3ccc(-c4c5c(cc6c4oc4ccccc46)-c4ccccc4C54c5ccccc5-c5ccccc54)cc3)cc2)cc1] |
| 3d | CAS 102113-98-4 | Int-4d | 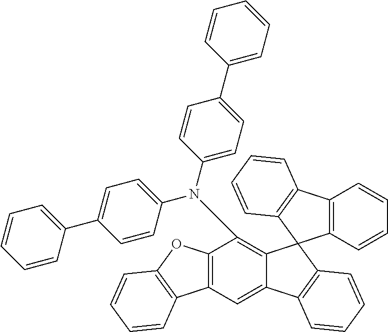 [SMILES: c1ccc(-c2ccc(N(c3ccc(-c4ccccc4)cc3)c3c4c(cc5c3oc3ccccc35)-c3ccccc3C43c4ccccc4-c4ccccc43)cc2)cc1] |
| 3e | CAS 102113-98-4 | Int-4e | 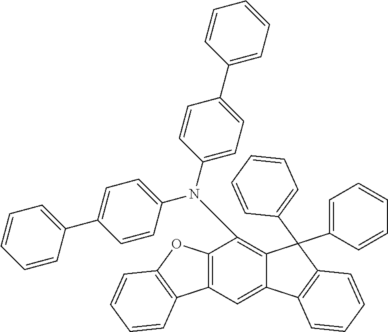 [SMILES: c1ccc(-c2ccc(N(c3ccc(-c4ccccc4)cc3)c3c4c(cc5c3oc3ccccc35)-c3ccccc3C4(c3ccccc3)c3ccccc3)cc2)cc1] |

### B) Device Examples

### 1) General Production Process for the OLEDs and Characterization of the OLEDs

Glass plates which have been coated with structured ITO (indium tin oxide) in a thickness of 50 nm form the substrates to which the OLEDs are applied.

The OLEDs basically have the following layer structure: substrate/hole injection layer (HIL)/hole transport layer (HTL1)/optional second hole transport layer (HTL2)/electron blocker layer (EBL)/emission layer (EML)/optional hole blocker layer (HBL)/electron transport layer (ETL1)/optional second electron transport layer (ETL2)/electron injection layer (EIL) and finally a cathode. The cathode is formed by an aluminum layer of thickness 100 nm. The exact structure of the OLEDs can be found in the tables which follow. The materials required for production of the OLEDs are shown in a table below.

All materials are applied by thermal vapor deposition in a vacuum chamber. In this case, the emission layer consists of at least one matrix material (host material) and an emitting dopant (emitter) which is added to the matrix material(s) in a particular proportion by volume by co-evaporation. Details given in such a form as H:SEB (95%:5%) mean here that the material H is present in the layer in a proportion by volume of 95% and SEB in a proportion of 5%. In an analogous manner, the electron transport layer and the hole injection layer also consist of a mixture of two materials. The structures of the materials that are used in the OLEDs are shown in Table 7. Compound HTM-B which is likewise used is a 2-aminofluorene that bears a substituent on one of the aromatic six-membered rings of the fluorene. Compound EBM-B which is likewise used contains an amino group and a 1-spirobifluorenyl group.

The OLEDs are characterized in a standard manner. For this purpose, the electroluminescence spectra, the external quantum efficiency (EQE, measured in %) as a function of the luminance, calculated from current-voltage-luminance characteristics assuming Lambertian radiation characteristics, and the lifetime are determined. The parameter EQE @ 10 mA/cm² refers to the external quantum efficiency which is attained at 10 mA/cm². The lifetime LT is defined as the time after which the luminance drops from the starting luminance to a certain proportion in the course of operation with constant current density. An LT90 figure means here that the lifetime reported corresponds to the time after which the luminance has dropped to 90% of its starting value. The figure @60 mA/cm² means here that the lifetime in question is measured at 60 mA/cm².

### 2) Use as Electron Blocker in a Blue-Fluorescing OLED:

OLEDs are produced with the following structure:

TABLE 1
OLED structure

| | HIL | HTL1 | HTL2 | EBL | EML | ETL1 | EIL |
|---|---|---|---|---|---|---|---|
| | Thickness/ | Thickness/ | Thickness/ | Thickness/ | Thickness/ | Thickness/ | Thickness/ |
| Ex | nm | nm | nm | nm | nm | nm | nm |
| 1 | HTM: | HTM | HTM-1: | HTM-1 | H:SEB | ETM:LiQ | LiQ |
| | p-dopant | 160 nm | p-dopant | 10 nm | (95%:5%) | (50:50%) | 1 nm |
| | (5%) | | (5%) | | 20 nm | 30 nm | |
| | 20 nm | | 20 nm | | | | |
| 2 | HTM: | HTM | HTM-2: | HTM-2 | H:SEB | ETM:LiQ | LiQ |
| | p-dopant | 160 nm | p-dopant | 10 nm | (95%:5%) | (50:50%) | 1 nm |
| | (5%) | | (5%) | | 20 nm | 30 nm | |
| | 20 nm | | 20 nm | | | | |
| 3 | HTM: | HTM | | HTM-1 | H:SEB | ETM:LiQ | LiQ |
| | p-dopant | 180 nm | | 10 nm | (95%:5%) | (50:50%) | 1 nm |
| | (5%) | | | | 20 nm | 30 nm | |
| | 20 nm | | | | | | |
| 4 | HTM: | HTM | — | HTM-2 | H:SEB | ETM:LiQ | LiQ |
| | p-dopant | 180 nm | | 10 nm | (95%:5%) | (50:50%) | 1 nm |
| | (5%) | | | | 20 nm | 30 nm | |
| | 20 nm | | | | | | |

OLEDs 1 to 4 show that the compounds of the invention are of excellent suitability as materials in OLEDs. The OLEDs show very good properties as hole transport materials, particularly in an EBL layer, where they lead in particular to very high external quantum efficiencies and a low operating voltage and very good lifetime.

The OLEDs have good results for lifetime, efficiency and operating voltage, as shown in the following table:

TABLE 2
OLED data

| | V (at 10 | EQE @ | LT90 (at |
|---|---|---|---|
| Ex. | mA/cm²) | 10 mA/cm² | 60 mA/cm²) |
| 1 | 3.7 | 8.4 | 110 |
| 2 | 3.7 | 7.5 | 140 |
| 3 | 3.9 | 9.0 | 100 |
| 4 | 3.9 | 8.7 | 120 |

### 3) Examples for Use in the Electron Blocker Layer of a Green-Phosphorescing OLED

OLEDs are produced with the following structure:

TABLE 3
OLED structure

| | HIL | HTL1 | EBL | EML | ETL1 | ETL2 | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| 5 | HTM:p- | HTM | HTM-1 | TMM-1 | ETM | ETM:LiQ | LiQ |
| | dopant | 220 nm | 10 nm | (59%): | 10 nm | (50:50%) | 1 nm |
| | (5%) | | | TMM-2 | | 30 nm | |
| | 20 nm | | | (29%): | | | |
| | | | | TEG(12%) | | | |
| | | | | 30 nm | | | |
| 6 | HTM:p- | HTM | HTM-2 | TMM-1 | ETM | ETM:LiQ | LiQ |
| | dopant | 220 nm | 10 nm | (59%): | 10 nm | (50:50%) | 1 nm |
| | (5%) | | | TMM-2 | | 30 nm | |
| | 20 nm | | | (29%): | | | |
| | | | | TEG(12%) | | | |
| | | | | 30 nm | | | |

OLEDs 5 and 6 show that the compounds of the invention are of excellent suitability as electron blocker for green-fluorescing OLEDs. The OLEDs show very good properties as hole transport or electron blocker materials, where they lead in particular to low operating voltages with good external quantum efficiencies and excellent lifetime.

The OLEDs have good results for lifetime, efficiency and operating voltage, as shown in the following table:

TABLE 4
OLED data

| | V (at 2 | EQE @ | LT90 (at |
|---|---|---|---|
| Ex. | mA/cm²) | 10 mA/cm² | 40 mA/cm²) |
| 5 | 3.9 | 17.4 | 130 |
| 6 | 4.0 | 17.8 | 110 |

### 4) Example for Use as Hole Transport Layer of a Blue-Fluorescing OLED

An OLED is produced with the following structure:

TABLE 5
OLED structure

| | HIL | HTL1 | EBL | EML | ETL1 | EIL |
|---|---|---|---|---|---|---|
| | Thick- | Thickness/ | Thick- | Thickness/ | Thickness / | Thick- |
| Ex. | ness/nm | nm | ness/nm | nm | nm | ness/nm |
| 7 | HTM-1: | HTM-1 | EBM | H:SEB | ETM:LiQ | LiQ |
| | p-dopant | 180 nm | 10 nm | (95%:5%) | (50:50%) | 1 nm |
| | (5%) | | | 20 nm | 30 nm | |
| | 20 nm | | | | | |

The example (OLED 7) shows that the inventive compound HTM-1 is of excellent suitability as hole transport material for blue-fluorescing OLEDs. The OLED shows very good properties as hole transport material and low operating voltage with good external quantum efficiencies and excellent lifetime.

The OLED has good results for lifetime, efficiency and operating voltage, as shown in the following table:

TABLE 6
OLED data

| | V (at 10 | EQE @ | LT90 (at |
|---|---|---|---|
| Ex. | mA/cm²) | 10 mA/cm² | 60 mA/cm²) |
| 7 | 4.6 | 8.6 | 120 |

HTM-2 is also usable in a stack as shown in table 5, with good OLED performance.

### 5) Furthermore, the Following Blue-Fluorescing OLEDs Containing a Compound of the Invention are Produced in the EBL:

TABLE
OLED structure

| | HIL | HTL1 | EBL | EML | ETL1 | ETL2 | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| 8 | HTM-B:p- | HTM-B | HTM-1 | H-2:SEB-2 | HBM | ETM-2: | LiQ |
| | dopant (5%) | 185 nm | 5 nm | (95%:5%) | 5 nm | LiQ | 1 nm |
| | 10 nm | | | 20 nm | | (50:50%) | |
| | | | | | | 30 nm | |
| 9 | HTM-B:p- | HTM-B | HTM-2 | H-2:SEB-2 | HBM | ETM-2: | LiQ |
| | dopant (5%) | 185 nm | 5 nm | (95%:5%) | 5 nm | LiQ | 1 nm |
| | 10 nm | | | 20 nm | | (50:50%) | |
| | | | | | | 30 nm | |

OLEDs 8 and 9 show that the compounds of the invention are of excellent suitability as electron blocker materials for blue-fluorescing OLEDs. The OLEDs achieve a low operating voltage with good external quantum efficiency and good lifetime:

TABLE
OLED data

| | V (at 10 | EQE @ | LT80 (at |
|---|---|---|---|
| Ex. | mA/cm²) | 10 mA/cm² | 60 mA/cm²) |
| 8 | 3.8 | 9.1% | 250 |
| 9 | 3.9 | 9.2% | 240 |

### 6) Furthermore, the Following Green-Phosphorescing OLEDs Containing an Inventive Compound HTM-1, HTM-2 or HTM-3 in the EBL are Produced:

TABLE
OLED structure

| | HIL | HTL1 | EBL | EML | ETL1 | ETL2 | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| 10 | HTM-B: | HTM-B | HTM-1 | TMM-3 | HBM | ETM:LiQ | LiQ |
| | p-dopant | 50 nm | 30 nm | (32%): | 5 nm | (50:50%) | 1 nm |
| | (3%) | | | TMM-4 | | 30 nm | |
| | 10 nm | | | (60%):TEG | | | |
| | | | | (8%) | | | |
| | | | | 35 nm | | | |
| 11 | HTM-B: | HTM-B | HTM-2 | TMM-3 | HBM | ETM:LiQ | LiQ |
| | p-dopant | 50 nm | 30 nm | (32%): | 5 nm | (50:50%) | 1 nm |
| | (3%) | | | TMM-4 | | 30 nm | |
| | 10 nm | | | (60%):TEG | | | |
| | | | | (8%) | | | |
| | | | | 35 nm | | | |
| 12 | HTM-B: | HTM-B | HTM-3 | TMM-3 | HBM | ETM:LiQ | LiQ |
| | p-dopant | 50 nm | 30 nm | (32%): | 5 nm | (50:50%) | 1 nm |
| | (3%) | | | TMM-4 | | 30 nm | |
| | 10 nm | | | (60%):TEG | | | |
| | | | | (8%) | | | |
| | | | | 35 nm | | | |

OLEDs 10-12 show that the compounds of the invention are of excellent suitability as materials in the electron blocker layer of green-phosphorescing OLEDs. The OLEDs achieve a low operating voltage with good external quantum efficiency and excellent lifetime:

TABLE
OLED data

| | V (at 10 | EQE @ | LT80 (at |
|---|---|---|---|
| Ex. | mA/cm²) | 10 mA/cm² | 80 mA/cm² |
| 10 | 4.0 | 23.1% | 260 h |
| 11 | 4.3 | 23.0% | 260 h |
| 12 | 4.0 | 24.1% | 200 h |

### 7) in Addition, the Following Blue-Fluorescing OLED Containing the Inventive Compound HTM-3 in the HTL is Produced:

| | HIL | HTL1 | EBL | EML | ETL1 | ETL2 | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| 13 | HTM-B: | HTM-3 | EBM-B | H-2:SEB- | HBM | ETM- | LiQ |
| | p-dopant | 185 nm | 5 nm | 2 | 5 nm | 2:LiQ | 1 nm |
| | (5%) | | | (95%:5%) | | (50:50%) | |
| | 10 nm | | | 20 nm | | 30 nm | |

This OLED (example 13) shows that the inventive compound HTM-3 is suitable as hole transport material for blue-fluorescing OLEDs. The OLED features very good efficiency and good voltage and lifetime:

TABLE
OLED data

| | V (at 10 | EQE @ | LT80 (at |
|---|---|---|---|
| Ex. | mA/cm²) | 10 mA/cm² | 60 mA/cm²) |
| 13 | 8.1 | 10.4% | 60 h |

8) Furthermore, Compounds HTM-1 and HTM-2 are Used as Hole Transport Material for a Blue-Fluorescing OLED and Compared with Each of Compounds CE-1 and CE-2 in an Otherwise Identical Stack Structure:

TABLE
OLED structure

| | HIL | HTL1 | EBL | EML | ETL1 | ETL2 | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| 14 | HTM-B: | HTM-1 | EBM-B | H-2:SEB- | HBM | ETM- | LiQ |
| | p-dopant | 185 nm | 5 nm | 2 | 5 nm | 2:LiQ | 1 nm |
| | (5%) | | | (95%:5%) | | (50:50%) | |
| | 10 nm | | | 20 nm | | 30 nm | |
| 15 | HTM-B: | HTM-2 | EBM-B | H-2:SEB- | HBM | ETM- | LiQ |
| | p-dopant | 185 nm | 5 nm | 2 | 5 nm | 2:LiQ | 1 nm |
| | (5%) | | | (95%:5%) | | (50:50%) | |
| | 10 nm | | | 20 nm | | 30 nm | |
| Comp. 1 | HTM-B: | CE-1 | EBM-B | H-2:SEB- | HBM | ETM- | LiQ |
| | p-dopant | 185 nm | 5 nm | 2 | 5 nm | 2:LiQ | 1 nm |
| | (5%) | | | | | | |
| | 10 nm | | | (95%:5%) | | (50:50%) | |
| | | | | 20 nm | | 30 nm | |
| Comp.2 | HTM-B: | CE-2 | EBM-B | H-2:SEB- | HBM | ETM- | LiQ |
| | p-dopant | 185 nm | 5 nm | 2 | 5 nm | 2:LiQ | 1 nm |
| | (5%) | | | (95%:5%) | | (50:50%) | |
| | 10 nm | | | 20 nm | | 30 nm | |

Examples 14 and 15 show that the compounds of the invention are of excellent suitability as hole transport materials for blue-fluorescing OLEDs. The OLEDs achieve a low operating voltage with good external quantum efficiency and excellent lifetime (see below). Example 14 by comparison with experiment Comp. 1 shows that the inventive compound HTM-1 leads to lower operating voltage, higher efficiency and longer lifetime of the OLED than the comparative compound CE-1. Example 15 by comparison with experiment Comp.2 shows that the inventive compound HTM-2 here too leads to lower operating voltage, higher efficiency and longer lifetime of the OLED than compound CE-2.

TABLE
OLED data

| | V (at 10 | EQE @ | LT80 (at |
|---|---|---|---|
| Ex. | mA/cm²) | 10 mA/cm² | 60 mA/cm²) |
| 14 | 5.2 | 8.6% | 240 h |
| 15 | 7.7 | 8.6% | 200 h |
| Comp.1 | 8.1 | 7.5% | 16 h |
| Comp.2 | 12.5 | 7.1% | 13 h |

TABLE 7

Materials used

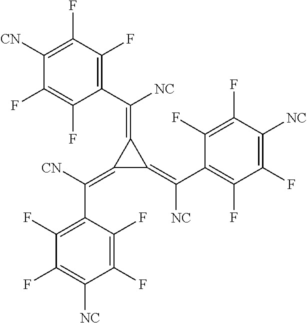

p-Dopant

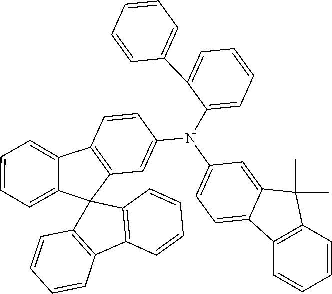 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

HTM

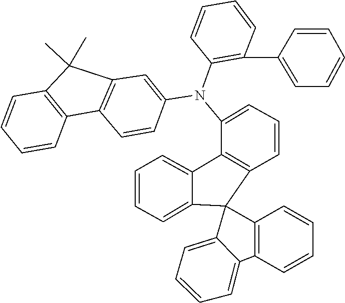 [SMILES: CC1(C)c2ccccc2-c2ccc(N(c3ccccc3-c3ccccc3)c3cccc4c3-c3ccccc3C43c4ccccc4-c4ccccc43)cc21]

EBM

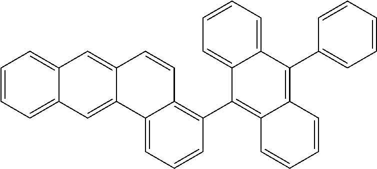 [SMILES: c1ccc(-c2c3ccccc3c(-c3cccc4c3ccc3cc5ccccc5cc34)c3ccccc23)cc1]

H

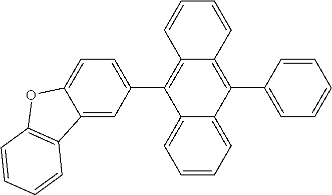 [SMILES: c1ccc(-c2c3ccccc3c(-c3ccc4oc5ccccc5c4c3)c3ccccc23)cc1]

H-2

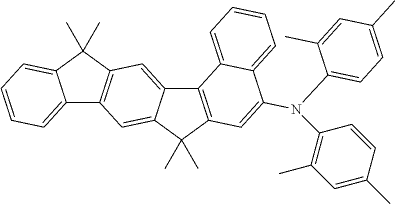 [SMILES: Cc1ccc(N(c2ccc(C)cc2C)c2cc3c(c4ccccc24)-c2cc4c(cc2C3(C)C)-c2ccccc2C4(C)C)c(C)c1]

SEB

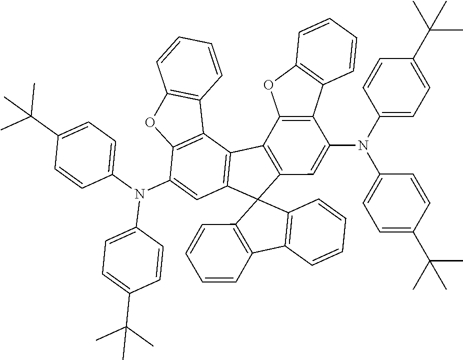 [SMILES: CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2oc2ccccc24)C32c3ccccc3-c3ccccc32)cc1]

SEB-2

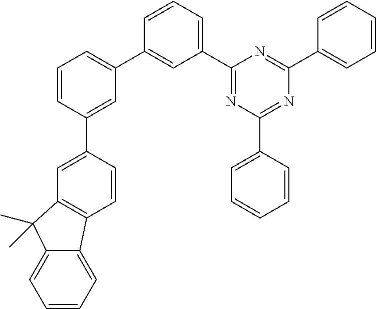 [SMILES: CC1(C)c2ccccc2-c2ccc(-c3cccc(-c4cccc(-c5nc(-c6ccccc6)nc(-c6ccccc6)n5)c4)c3)cc21]

HBM

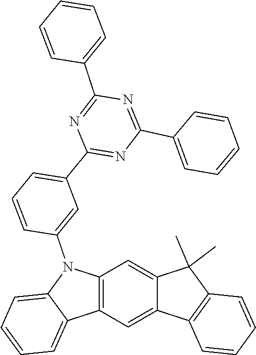 [SMILES: CC1(C)c2ccccc2-c2cc3c4ccccc4n(-c4cccc(-c5nc(-c6ccccc6)nc(-c6ccccc6)n5)c4)c3cc21]

TMM-1

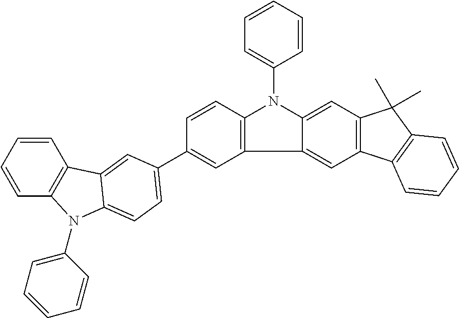 [SMILES: CC1(C)c2ccccc2-c2cc3c4cc(-c5ccc6c(c5)c5ccccc5n6-c5ccccc5)ccc4n(-c4ccccc4)c3cc21]

TMM-2

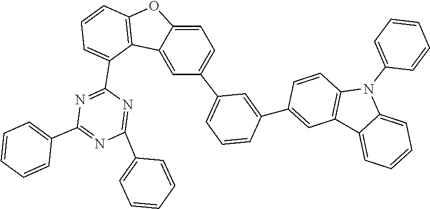 [SMILES: c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1]

TMM-3

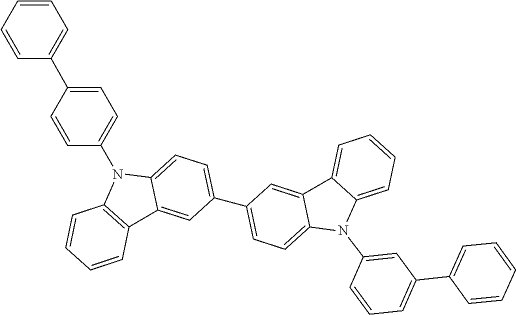 [SMILES: c1ccc(-c2ccc(-n3c4ccccc4c4cc(-c5ccc6c(c5)c5ccccc5n6-c5cccc(-c6ccccc6)c5)ccc43)cc2)cc1]

TMM-4

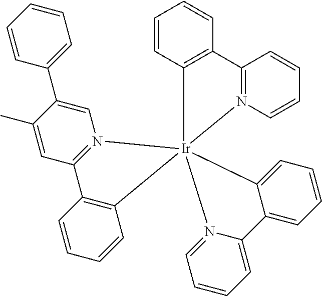

TEG

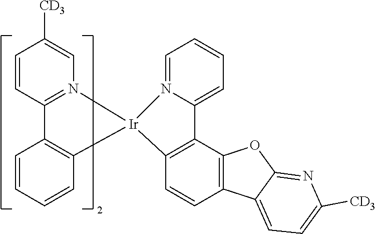

TEG-2

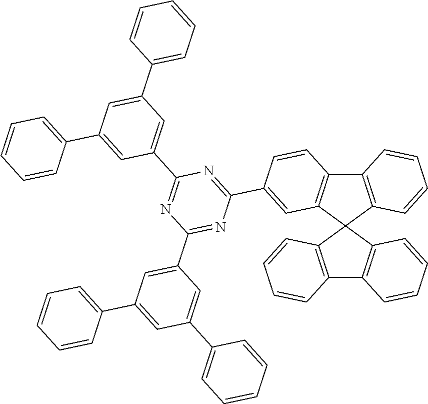 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ETM

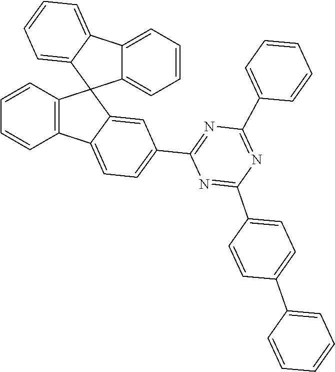 [SMILES: c1ccc(-c2ccc(-c3nc(-c4ccccc4)nc(-c4ccc5c(c4)C4(c6ccccc6-c6ccccc64)c4ccccc4-5)n3)cc2)cc1]

ETM-2

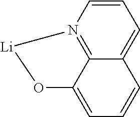

LiQ

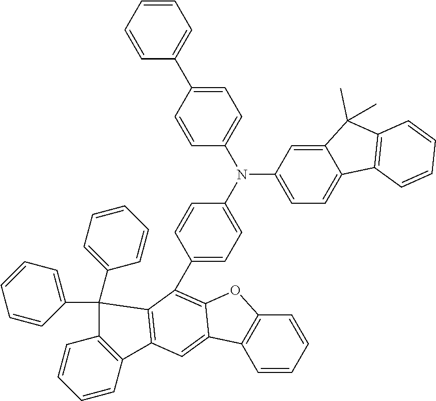 [SMILES: CC1(C)c2ccccc2-c2ccc(N(c3ccc(-c4ccccc4)cc3)c3ccc(-c4c5c(cc6c4oc4ccccc46)-c4ccccc4C5(c4ccccc4)c4ccccc4)cc3)cc21]

HTM-1

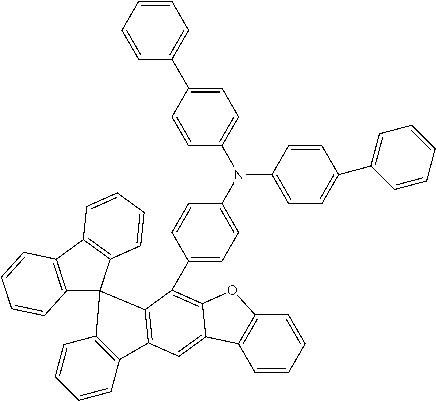 [SMILES: c1ccc(-c2ccc(N(c3ccc(-c4ccccc4)cc3)c3ccc(-c4c5c(cc6c4oc4ccccc46)-c4ccccc4C54c5ccccc5-c5ccccc54)cc3)cc2)cc1]

HTM-2

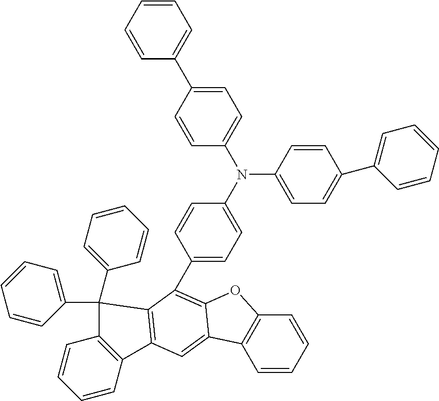 [SMILES: c1ccc(-c2ccc(N(c3ccc(-c4ccccc4)cc3)c3ccc(-c4c5c(cc6c4oc4ccccc46)-c4ccccc4C5(c4ccccc4)c4ccccc4)cc3)cc2)cc1]

HTM-3

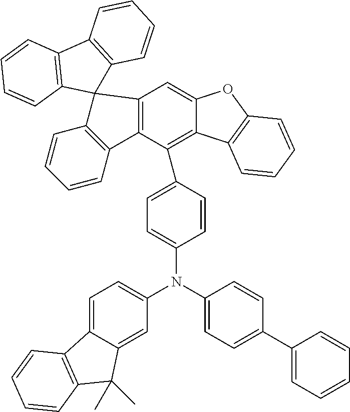 [SMILES: CC1(C)c2ccccc2-c2ccc(N(c3ccc(-c4ccccc4)cc3)c3ccc(-c4c5c(cc6oc7ccccc7c46)C4(c6ccccc6-c6ccccc64)c4ccccc4-5)cc3)cc21]

CE-1

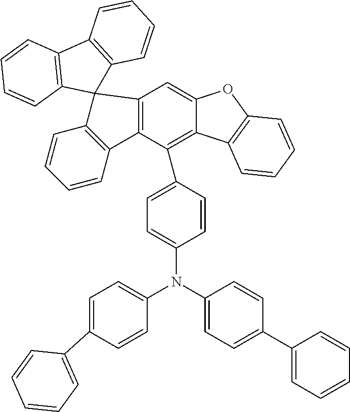 [SMILES: c1ccc(-c2ccc(N(c3ccc(-c4ccccc4)cc3)c3ccc(-c4c5c(cc6oc7ccccc7c46)C4(c6ccccc6-c6ccccc64)c4ccccc4-5)cc3)cc2)cc1]

CE-2

What is claimed is:

1. A compound of one of the following formulae: 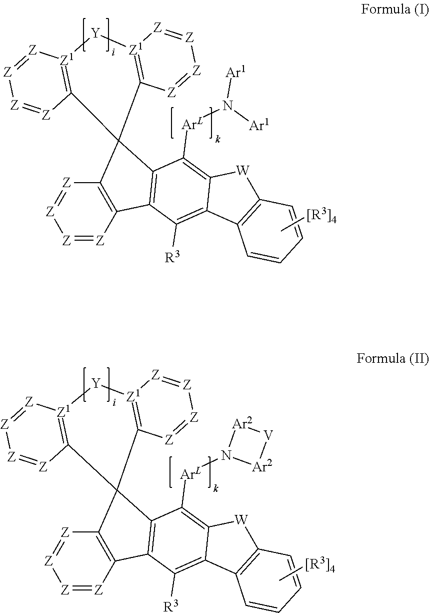
where:
W is the same or different at each instance and is selected from O and S;
Z is the same or different at each instance and is selected from CR¹ and N;
i is 0 or 1, where the Y group is absent when i=0;
when i=0, Z¹ is the same or different at each instance and is selected from CR¹ and N;
when i=1, Z¹ is C;
Y is the same or different at each instance and is selected from a bond, C(R¹)₂, O and S;
Ar^{L} is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
k is 0, 1, 2 or 3, where, when k=0, the Art group is absent and the two groups that bind to Ar^{L} in formula (I) and (II) are bonded directly to one another, where, when k=2, two Ar^{L} groups are bonded successively in a chain, and where, when k=3, three Ar^{L} groups are bonded successively in a chain;
Ar¹ is the same or different at each instance and is selected from aromatic ring systems which have 6 to 40 aromatic ring atoms and are substituted by R⁴ radicals, and heteroaromatic ring systems which have 5 to 40 aromatic ring atoms and are substituted by R⁴ radicals;
Ar² is the same or different at each instance and is selected from aromatic ring systems which have 6 to 40 aromatic ring atoms and are substituted by R⁵ radicals, and heteroaromatic ring systems which have 5 to 40 aromatic ring atoms and are substituted by R⁵ radicals, where the aromatic or heteroaromatic ring systems are joined in the ortho position to the V group and the nitrogen atom;
V is the same or different at each instance and is selected from a bond, O, S, Si(R⁵)₂ and C(R⁵)₂;
R¹ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R² is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C═CR⁶—, —C≡C—, Si(R⁶)₂, C—O, C═NR⁶, —C(═O)O—, —C(═O)NR⁶—, NR⁶, P(═O)(R⁶), —O—, —S—, SO or SO₂;
R³ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C═CR⁶—, —C≡C—, Si(R⁶)₂, C═O, C═NR⁶, —C(═O)O—, —C(═O)NR⁶, NR⁶, P(═O)(R⁶), —O—, —S—, SO or SO₂;
R⁴ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁴ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C═CR⁶—, —C≡C—, Si(R⁶)₂, C—O, C═NR⁶, —C(═O)O—, —C(═O)NR⁶, NR⁶, P(═O)(R⁶), —O—, —S—, SO or SO₂;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C═CR⁶—, —C≡C—, Si(R⁶)₂, C—O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, C(═O)R⁷, CN, Si(R⁷)₃, N(R⁷)₂, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁶ radicals may be joined to one another and may form a ring; where the alkyl, alkoxy, alkenyl and alkynyl groups mentioned and the aromatic ring systems and heteroaromatic ring systems mentioned are each substituted by R⁷ radicals; and where one or more CH₂ groups in the alkyl, alkoxy, alkenyl and alkynyl groups mentioned may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
R⁷ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁷ radicals may be joined to one another and may form a ring; and where the alkyl, alkoxy, alkenyl and alkynyl groups, aromatic ring systems and heteroaromatic ring systems mentioned may be substituted by one or more radicals selected from F and CN.

2. A compound as claimed in claim 1, characterized in that Z is CR¹.

3. A compound as claimed in claim 1, characterized in that Ar^{L} is the same or different at each instance and is selected from phenyl, biphenyl, naphthyl and fluorenyl, each substituted by R² radicals.

4. A compound as claimed in claim 1, characterized in that i is 1 and Y is a bond.

5. A compound as claimed in claim 1, characterized in that Ar¹ is the same or different at each instance and is selected from monovalent groups derived from benzene, biphenyl, terphenyl, quaterphenyl, naphthalene, fluorene, especially 9,9′-dimethylfluorene and 9,9′-diphenylfluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where each of the monovalent groups is substituted by R⁴ radicals.

6. A compound as claimed in claim 1, characterized in that Ar² is the same or different at each instance and is selected from ortho-divalent groups derived from benzene, biphenyl, terphenyl, quaterphenyl, naphthalene, fluorene, especially 9,9′-dimethylfluorene and 9,9′-diphenylfluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine and pyridazine, where each of the monovalent groups is substituted by R⁵ radicals.

7. A compound as claimed in claim 1, characterized in that, in the compounds of one of the formulae (I) and (II), zero, one, two or three of the R¹ groups per formula are not H or D, and in that these groups that are not H or D are the same or different at each instance and are selected from F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, where the alkyl groups mentioned, the aryl groups mentioned and the heteroaryl groups mentioned are each substituted by R⁶ radicals.

8. A compound as claimed in claim 1, characterized in that, in the compounds of one of the formulae (I) and (II), zero, one, two or three R³ groups per formula are not H or D, and in that these groups that are not H or D are the same or different at each instance and are selected from F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, where the alkyl groups mentioned, the aryl groups mentioned and the heteroaryl groups mentioned are each substituted by R⁶ radicals.

9. A compound as claimed in claim 1, characterized in that all R¹ and R³ radicals in formulae (I) and (II) are H or D.

10. A compound as claimed in claim 1, characterized in that zero, one, two, three or four R⁴ groups per Ar¹ radical are not H or D, and in that these groups that are not H or D are the same or different at each instance and are selected from F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, where the alkyl groups mentioned, the aryl groups mentioned and the heteroaryl groups mentioned are each substituted by R⁶ radicals.

11. A compound as claimed in claim 1, characterized in that zero, one, two, three or four R⁵ groups per Ar² radical are not H or D, and in that these groups that are not H or D are the same or different at each instance and are selected from F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, where the alkyl groups mentioned, the aryl groups mentioned and the heteroaryl groups mentioned are each substituted by R⁶ radicals.

12. A compound as claimed in claim 1, characterized in that the compound conforms to one of the following formulae: 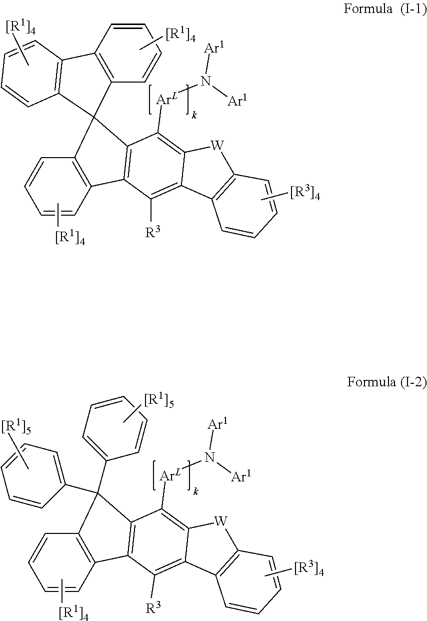
where the groups that occur are as defined in one or more of the preceding claims; or in that the compound conforms to one of the following formulae: 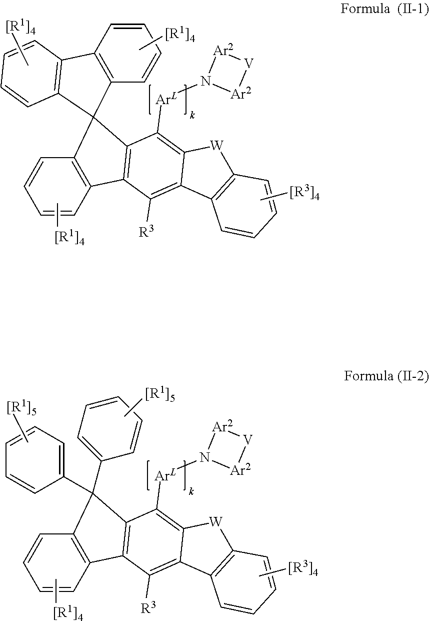
where the groups that occur are as defined in one or more of the preceding claims.

13. A compound as claimed in claim 1, characterized in that the compound conforms to one of the following formulae: 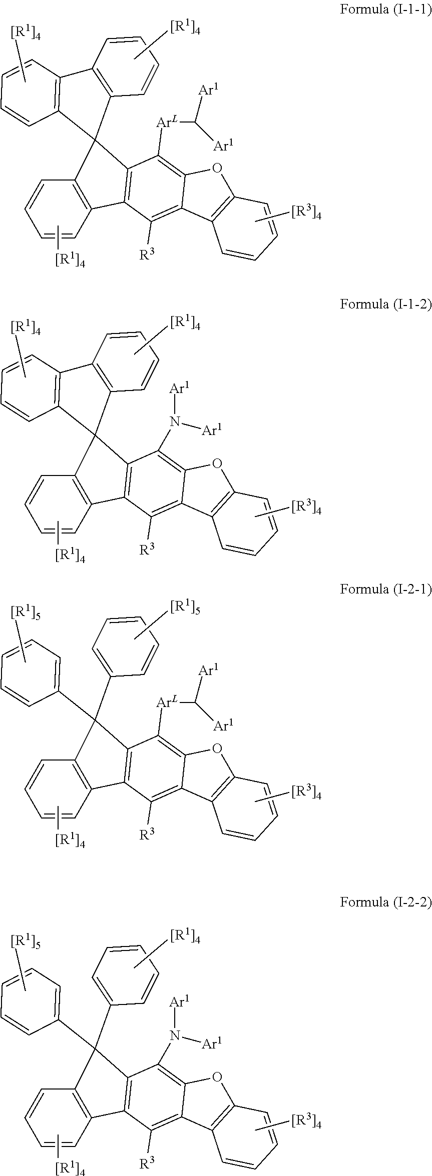
where the groups that occur are as defined in one or more of the preceding claims; or in that the compound conforms to one of the following formulae: 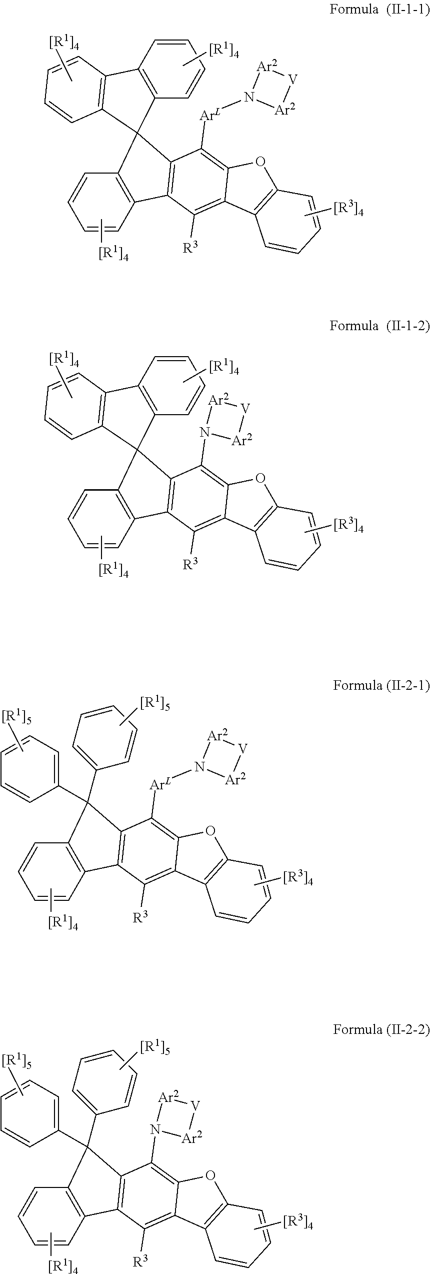
where the groups that occur are as defined in one or more of the preceding claims.

14. A process for preparing a compound as claimed in claim 1, characterized in that an indenodibenzofuran derivative a) substituted by a reactive group is reacted in a coupling reaction with a secondary amine, or b) is reacted in a coupling reaction with an aromatic or heteroaromatic species bearing a reactive group.

15. An oligomer, polymer or dendrimer containing one or more compounds as claimed in claim 1, wherein the bond(s) to the polymer, oligomer or dendrimer may be localized at any desired positions substituted by R¹, R², R³, R⁴ or R⁵ in formulae (I) and (II).

16. A formulation comprising at least one compound as claimed in claim 1 and at least one solvent.

17. An electronic device comprising at least one compound as claimed in claim 1.

18. The electronic device as claimed in claim 17, characterized in that it is an organic electroluminescent device and comprises an anode, cathode and at least one emitting layer, and in that the compound is present in a hole-transporting layer or in an emitting layer of the device.

19. An electronic device comprising at least one polymer, oligomer or dendrimer as claimed in claim 15 and at least one solvent.

20. A formulation comprising at least one polymer, oligomer or dendrimer as claimed in claim 15 and at least one solvent.
